SEQUENCE LISTING (140 sequences)

SEQ ID NO: 1 - protein, 166 aa, Synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 2 - protein, 276 aa, Burkholderia cenocepacia
GSYALGPYQISAPQLPAYNGQTVGTFYYVNDAGGLESKVFSSGGPTPYPNYANAGHVEGQSALFMRDNGISEGLGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGCVFHNNPEGTCGFCVNMTETLLPENAKMTVVPPEGAIPVKRGATGETKVFTGNSNSPKSPTKGGC

SEQ ID NO: 3 - protein, 44 aa, Synthetic construct
GSYALGPYQISAPQLPAYNGQTVGTFYYVNDAGGLESKVFSSGG

SEQ ID NO: 4 - protein, 94 aa, Synthetic construct
PTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 5 - protein, 108 aa, Synthetic construct
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 6 - protein, 30 aa, Synthetic construct
AIPVKRGATGETKVFTGNSNSPKSPTKGGC

SEQ ID NO: 7 - protein, 107 aa, Synthetic construct
YKCPECGKSFSSKKALTEHQRTHTGEKPYKCPECGKSFSTHLDLIRHQRTHTGEKPYKCPECGKSFSHTGHLLEHQRTHTGEKPFECKDCGKAFIQKSNLIRHQRTH

SEQ ID NO: 8 - protein, 139 aa, Synthetic construct
YSCGICGKSFSDSSAKRRHCILHTGEKPYKCPECGKSFSSPADLTRHQRTHLRQKDGERPYKCPECGKSFSTHLDLIRHQRTHTGEKPYKCPECGKSFSHTGHLLEHQRTHTGEKPFECKDCGKAFIQKSNLIRHQRTH

SEQ ID NO: 9 - protein, 170 aa, Synthetic construct
YKCPECGKSFSTHLDLIRHQRTHTGEKPYKCPECGKSFSHTGHLLEHQRTHTGEKPFECKDCGKAFIQKSNLIRHQRTHLRQKDGGGSERPYKCPECGKSFSTHLDLIRHQRTHTGEKPYKCDECGKNFTQSSNLIVHKRIHTGEKPYKCPECGKSFSTHLDLIRHQRTH

SEQ ID NO: 10 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 11 - protein, 747 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 12 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQRLLPVLCQAHGLTPAQVVAIASNNGGKQALETVQRLLPVLCQDHGLTPDQVVAIASNNGGKQALETVQRLLPVLCQDHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQDHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 13 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 14 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPDQVVAIASHDGGKQALETVQRLLPVLCQAHGLTPAQVVAIASNGGGKQALETVQRLLPVLCQDHGLTPAQVVAIASNGGGKQALETVQRLLPVLCQDHGLTPEQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 15 - protein, 712 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLLV

SEQ ID NO: 16 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLV

SEQ ID NO: 17 - protein, 745 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQRLLPVLCQAHGLTPAQVVAIASNNGGKQALETVQRLLPVLCQDHGLTPDQVVAIASNNGGKQALETVQRLLPVLCQDHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQDHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLV

SEQ ID NO: 18 - protein, 679 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 19 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 20 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 21 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 22 - protein, 678 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 23 - protein, 712 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 24 - protein, 745 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALESIVAQLSRPDPALAALTNDHLVALACLGGRPALDAVKKGLLV

SEQ ID NO: 25 - protein, 745 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 26 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 27 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALETVQRLLPVLCQAHGLTPAQVVAIASNNGGKQALETVQRLLPVLCQDHGLTPEQVVAIASNGGGKQALESIVAQLSRPDPALAALTNDHLVALACLGGRPALDAVKKGLLV

SEQ ID NO: 28 - protein, 746 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNNGGKQALETVQRLLPVLCQAHGLTPDQVVAIASNGGGKQALETVQRLLPVLCQAHGLTPAQVVAIASNNGGKQALETVQRLLPVLCQAHGLTPAQVVAIASNIGGKQALETVQRLLPVLCQAHGLTPEQVVAIASNGGGKQALESIVAQLSRPDPALAALTNDHLVALACLGGRPALDAVKKGLLV

SEQ ID NO: 29 - protein, 745 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 30 - protein, 745 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 31 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNGGGKQALESIVAQLSRPDPALAALTNDHLVALACLGGRPALDAVKKGLGGS

SEQ ID NO: 32 - protein, 814 aa, Synthetic construct; identical to SEQ ID NO: 31

SEQ ID NO: 33 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNGGGKQALESIVAQLSRPDPALAALTNDHLVALACLGGRPALDAVKKGLGGS

SEQ ID NO: 34 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLGGS

SEQ ID NO: 35 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQAHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 36 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPEQVVAIASNGGGKQALESIVAQLSRPDPALAALTNDHLVALACLGGRPALDAVKKGLGGS

SEQ ID NO: 37 - protein, 815 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 38 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPEQVVAIASNGGGKQALESIVAQLSRPDPALAALTNDHLVALACLGGRPALDAVKKGLGGS

SEQ ID NO: 39 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 40 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLGGS

SEQ ID NO: 41 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPEQVVAIASNGGGKQALESIVAQLSRPDPALAALTNDHLVALACLGGRPALDAVKKGLLV

SEQ ID NO: 42 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLV

SEQ ID NO: 43 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 44 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPEQVVAIASNGGGKQALESIVAQLSRPDPALAALTNDHLVALACLGGRPALDAVKKGLGGS

SEQ ID NO: 45 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPEQVVAIASNGGGKQALESIVAQLSRPDPALAALTNDHLVALACLGGRPALDAVKKGLGGS

SEQ ID NO: 46 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLLV

SEQ ID NO: 47 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 48 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 49 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQAHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNGGGKQALESIVAQLSRPDPALAALTNDHLVALACLGGRPALDAVKKGLLV

SEQ ID NO: 50 - protein, 814 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALESIVAQLSRPDPALAALTNDHLVALACLGGRPALDAVKKGLLV

SEQ ID NO: 51 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLV

SEQ ID NO: 52 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQAHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLLV

SEQ ID NO: 53 - protein, 813 aa, Synthetic construct
GIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPEQVVAIASNGGGKQALESIVAQLSRPDPALAALTNDHLVALACLGGRPALDAVKKGLLV

SEQ ID NO: 54 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPAQVVAIASNNGGKQALETVQRLLPVLCQAHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 55 - protein, 679 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNIGGKQALETVQRLLPVLCQAHGLTPEQVVAIASNGGGKQALESIVAQLSRPDPALAALTNDHLVALACLGGRPALDAVKKGLGGS

SEQ ID NO: 56 - protein, 747 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASNGGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPEQVVAIASHDGGKQALETVQRLLPVLCQAHGLTPAQVVAIASHDGGKQALETVQRLLPVLCQDHGLTPDQVVAIASNIGGKQALETVQRLLPVLCQAHGLTPEQVVAIASNGGGKQALESIVAQLSRPDPALAALTNDHLVALACLGGRPALDAVKKGLGGS

SEQ ID NO: 57 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASNGGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPEQVVAIASHDGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGS

SEQ ID NO: 58 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPDQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 59 - protein, 747 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 60 - protein, 781 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 61 - protein, 747 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 62 - protein, 815 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 63 - protein, 781 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 64 - protein, 747 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQDHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 65 - protein, 713 aa, Synthetic construct
GIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQDHGLTPDQVVAIASNNGGKQALETVQRLLPVLCQAHGLTPEQVVAIASNGGGKQALESIVAQLSRPDPALAALTNDHLVALACLGGRPALDAVKKGLGGS

SEQ ID NO: 66 - protein, 970 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPAQVVAIASNNGGKQALETVQRLLPVLCQAHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 67 - protein, 936 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNIGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 68 - protein, 918 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASNGGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPEQVVAIASHDGGKQALETVQRLLPVLCQAHGLTPAQVVAIASHDGGKQALETVQRLLPVLCQDHGLTPDQVVAIASNIGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 69 - protein, 884 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASNGGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASNGGGKQALETVQRLLPVLCQDHGLTPEQVVAIASHDGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 70 - protein, 884 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPDQVVAIASNNGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 71 - protein, 918 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 72 - protein, 952 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 73 - protein, 918 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGSGSAIPVKRGATGETKVFIGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 74 - protein, 1072 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 75 - protein, 1038 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 76 - protein, 1004 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQDHGLTPAQVVAIASNNGGKQALETVQRLLPVLCQAHGLTPAQVVAIASNNGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 77 - protein, 970 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPAQVVAIASNIGGKQALETVQRLLPVLCQDHGLTPDQVVAIASNNGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEGSGGSTNLSDIIEKETGKQLVIQESILMLPEEVEEVIGNKPESDILVHTAYDESTDENVMLLTSDAPEYKPWALVIQDSNGENKIKML

SEQ ID NO: 78 - protein, 883 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 79 - protein, 917 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 80 - protein, 916 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQRLLPVLCQAHGLTPAQVVAIASNNGGKQALETVQRLLPVLCQDHGLTPDQVVAIASNNGGKQALETVQRLLPVLCQDHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQDHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 81 - protein, 916 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 82 - protein, 916 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPDQVVAIASHDGGKQALETVQRLLPVLCQAHGLTPAQVVAIASNGGGKQALETVQRLLPVLCQDHGLTPAQVVAIASNGGGKQALETVQRLLPVLCQDHGLTPEQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 83 - protein, 978 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 84 - protein, 1012 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 85 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASHDGGKQALETVQRLLPVLCQAHGLTPAQVVAIASNNGGKQALETVQRLLPVLCQDHGLTPDQVVAIASNNGGKQALETVQRLLPVLCQDHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNIGGKQALETVQRLLPVLCQAHGLTPAQVVAIASHDGGKQALETVQRLLPVLCQDHGLTPAQVVAIASHDGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 86 - protein, 849 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 87 - protein, 883 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 88 - protein, 916 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 89 - protein, 916 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 90 - protein, 944 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 91 - protein, 978 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 92 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 93 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQAHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQDHGLTPDQVVAIASNIGGKQALETVQRLLPVLCQDHGLTPAQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 94 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNNGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 95 - protein, 1012 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 96 - protein, 1012 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASNNGGKQALETVQRLLPVLCQAHGLTPDQVVAIASNNGGKQALETVQRLLPVLCQDHGLTPEQVVAIASNNGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQAHGLTPAQVVAIASNNGGKQALETVQRLLPVLCQAHGLTPEQVVAIASNGGGKQALETVQRLLPVLCQAHGLTPAQVVAIASNNGGKQALETVQRLLPVLCQDHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 97 - protein, 1012 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNNGGKQALETVQRLLPVLCQAHGLTPAQVVAIASNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNNGGKQALETVQRLLPVLCQAHGLTPDQVVAIASNGGGKQALETVQRLLPVLCQAHGLTPAQVVAIASNNGGKQALETVQRLLPVLCQAHGLTPAQVVAIASNIGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 98 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNGGGKQALETVQRLLPVLCQAHGLTPDQVVAIASHDGGKQALETVQRLLPVLCQDHGLTPDQVVAIASNGGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPEQVVAIASNNGGKQALETVQRLLPVLCQAHGLTPDQVVAIASNGGGKQALETVQRLLPVLCQDHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 99 - protein, 1011 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNNGGKQALETVQRLLPVLCQDHGLTPDQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 100 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 101 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 102 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 103 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQAHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 104 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 105 - protein, 985 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 106 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPEQVVAIASHDGGKQALETVQRLLPVLCQAHGLTPAQVVAIASNNGGKQALETVQRLLPVLCQAHGLTPAQVVAIASNIGGKQALETVQRLLPVLCQAHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 107 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 108 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 109 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASHDGGKQALETVQRLLPVLCQAHGLTPDQVVAIASNNGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 110 - protein, 1080 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQAHGLTPAQVVAIASNNGGKQALETVQRLLPVLCQAHGLTPDQVVAIASNIGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNIGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 111 - protein, 329 aa, Synthetic construct
MLGFVGRVAAAPASGALRRLTPSASLPPAQLLLRAAPTAVHPVRDYAAQYPYDVPDYAVDEMTKKFGTLTIHDTEKAAEFGIHGVPAAMGGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGTPHEVGVYTLSGTPHEVGVYTLYKCPECGKSFSSKKALTEHQRTHTGEKPYKCPECGKSFSTHLDLIRHQRTHTGEKPYKCPECGKSFSHTGHLLEHQRTHTGEKPFECKDCGKAFIQKSNLIRHQRTH

SEQ ID NO: 112 - protein, 361 aa, Synthetic construct
MLGFVGRVAAAPASGALRRLTPSASLPPAQLLLRAAPTAVHPVRDYAAQYPYDVPDYAVDEMTKKFGTLTIHDTEKAAEFGIHGVPAAMGGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGTPHEVGVYTLSGTPHEVGVYTLYSCGICGKSFSDSSAKRRHCILHTGEKPYKCPECGKSFSSPADLTRHQRTHLRQKDGERPYKCPECGKSFSTHLDLIRHQRTHTGEKPYKCPECGKSFSHTGHLLEHQRTHTGEKPFECKDCGKAFIQKSNLIRHQRTH

SEQ ID NO: 113 - protein, 392 aa, Synthetic construct
MLGFVGRVAAAPASGALRRLTPSASLPPAQLLLRAAPTAVHPVRDYAAQYPYDVPDYAVDEMTKKFGTLTIHDTEKAAEFGIHGVPAAMGGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGSGTPHEVGVYTLSGTPHEVGVYTLYKCPECGKSFSTHLDLIRHQRTHTGEKPYKCPECGKSFSHTGHLLEHQRTHTGEKPFECKDCGKAFIQKSNLIRHQRTHLRQKDGGGSERPYKCPECGKSFSTHLDLIRHQRTHTGEKPYKCDECGKNFTQSSNLIVHKRIHTGEKPYKCPECGKSFSTHLDLIRHQRTH

SEQ ID NO: 114 - protein, 498 aa, Synthetic construct
MLGFVGRVAAAPASGALRRLTPSASLPPAQLLLRAAPTAVHPVRDYAAQYPYDVPDYAVDEMTKKFGTLTIHDTEKAAEFGIHGVPAAMGGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGTPHEVGVYTLSGTPHEVGVYTLYKCPECGKSFSTHLDLIRHQRTHTGEKPYKCPECGKSFSHTGHLLEHQRTHTGEKPFECKDCGKAFIQKSNLIRHQRTHLRQKDGGGSERPYKCPECGKSFSTHLDLIRHQRTHTGEKPYKCDECGKNFTQSSNLIVHKRIHTGEKPYKCPECGKSFSTHLDLIRHQRTH

SEQ ID NO: 115 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 116 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 117 - protein, 984 aa, Synthetic construct
MALSRAVCGTSRQLAPVLGYLGSRQKHSLPDYPYDVPDYAGYPYDVPDYAGYPYDVPDYAGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPEQVVAIASNGGGKQALESIVAQLSRPDPALAALTNDHLVALACLGGRPALDAVKKGLGGSGSGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 118 - protein, 1080 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 119 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 120 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 121 - protein, 1080 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQAHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 122 - protein, 1080 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNGGGKQALETVQRLLPVLCQDHGLTPAQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPAQVVAIASNGGGKQALETVQRLLPVLCQAHGLTPDQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 123 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQDHGLTPDQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNNGGKQALETVQRLLPVLCQAHGLTPAQVVAIASNIGGKQALETVQRLLPVLCQAHGLTPDQVVAIASNGGGKQALETVQRLLPVLCQAHGLTPDQVVAIASNGGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 124 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASNNGGKQALETVQRLLPVLCQAHGLTPDQVVAIASNIGGKQALETVQRLLPVLCQDHGLTPDQVVAIASNIGGKQALETVQRLLPVLCQAHGLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQAHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 125 - protein, 1079 aa, Synthetic construct
MASVLTPLLLRGLTGSARRLPVPRAKIHSLDYKDHDGDYKDHDIDYKDDDDKGIRIQDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPEQVVAIASNGGGKQALESIVAQLSRPDPALAALTNDHLVALACLGGRPALDAVKKGLLVGSAIPVKRGATGETKVFTGNSNSPKSPTKGGCSGSETPGTSESATPESSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 126 - protein, 83 aa, Synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 127 - protein, 18 aa, Synthetic construct
VDEMTKKFGTLTIHDTEK

SEQ ID NO: 128 - protein, 31 aa, Synthetic construct
MALSRAVCGTSRQLAPVLGYLGSRQKHSLPD

SEQ ID NO: 129 - protein, 30 aa, Synthetic construct
MASVLTPLLLRGLTGSARRLPVPRAKIHSL

SEQ ID NO: 130 - protein, 49 aa, Synthetic construct
MLGFVGRVAAAPASGALRRLTPSASLPPAQLLLRAAPTAVHPVRDYAAQ

SEQ ID NO: 131 - protein, 16 aa, Synthetic construct
SGSETPGTSESATPES

SEQ ID NO: 132 - protein, 24 aa, Synthetic construct
SGTPHEVGVYTLSGTPHEVGVYTL

SEQ ID NO: 133 - protein, 14 aa, Synthetic construct
AAEFGIHGVPAAMG

SEQ ID NO: 134 - protein, 16 aa, Synthetic construct
AAEFGIHGVPAAMGGS

SEQ ID NO: 135 - protein, 4 aa, Synthetic construct
SGGS

SEQ ID NO: 136 - protein, 29 aa, Synthetic construct
YPYDVPDYAGYPYDVPDYAGYPYDVPDYA

SEQ ID NO: 137 - protein, 22 aa, Synthetic construct
DYKDHDGDYKDHDIDYKDDDDK

SEQ ID NO: 138 - protein, 9 aa, Synthetic construct
YPYDVPDYA

SEQ ID NO: 139 - protein, 108 aa, Synthetic construct
GSYALGPYQISAPQLPAYNGQTVGTFYYVNDAGGLESKVFISGGPTPYPNYVSAGHVEGQSALFMRDNGISEGLVFHNNPKGTCGFCVNMIETLLPENAAMTVVPPEG

SEQ ID NO: 140 - protein, 30 aa, Synthetic construct
AIPVKRGATGETKVFIGNSNSPKSPTKGGC

(19) United States
(12) Patent Application Publication
(10) Pub. No.: US 2026/0007775 A1
(43) Pub. Date: Jan. 8, 2026

(54) MITOCHONDRIAL BASE MUTATION EDITING SYSTEM FOR LEBER HEREDITARY OPTIC NEUROPATHY

(71) Applicant: EDGENE, INC., Incheon (KR)

(72) Inventors: Jin Soo KIM, Seoul (KR); Seong Hyun LEE, Seoul (KR); Chae Jin LIM, Seoul (KR)

(73) Assignee: EDGENE, INC., Incheon (KR)

(21) Appl. No.: 19/253,177

(22) Filed: Jun. 27, 2025

Related U.S. Application Data
(63) Continuation-in-part of application No. PCT/KR2023/021945, filed on Dec. 28, 2023.

(30) Foreign Application Priority Data
Dec. 29, 2022 (KR) 10-2022-0189900

(51) Int. Cl. A61K 48/00; C07K 14/195; C12N 9/78

(52) U.S. Cl. CPC A61K 48/0058; C07K 14/195; C12N 9/78; C12Y 305/04001; C12Y 305/04004; C07K 2319/81

(57) ABSTRACT

Described herein is a base editing system for correcting mutations G3460A, G11778A, or T14484C in mitochondrial DNA of a patient with Leber hereditary optic neuropathy (LHON) to a normal genotype. Also, described herein is a method for correcting a mutation in the mitochondrial genes of a patient with LHON to a normal genotype using a base editor that recognizes specific sites in the mitochondrial genes of the patient with LHON and has an activity of specifically correcting the adenine base at position 3460 or 11778, or the cytosine base at position 14484, by using a fusion protein or a polynucleotide encoding such a fusion protein. The base editor or nucleotide described herein may correct DNA mutations specific to LHON in a cellular or extracellular in vitro environment. Thus, described herein is also the use of the substance in the prevention or treatment of LHON.

### CROSS-REFERENCE TO RELATED APPLICATIONS

This application is a continuation-in-part application of and claims the benefit of priority to International Application No. PCT/KR2023/021945, filed on Dec. 28, 2023, which is based on and claims the benefit of priority to Korean Patent Application No. 10-2022-0189900, filed on Dec. 29, 2022 with the Korean Intellectual Property Office.

### REFERENCE TO APPENDIX [CD ROM/SEQUENCE LISTING]

This application contains a Sequence Listing XML which has been electronically submitted in.xml format. The xml file is named “520576.5000001_Sequence_Listing.xml” was created on Jan. 9, 2024 and is 216 bytes in size. The sequence listing contained in this .xml file is part of the specification and hereby incorporated by reference in its entirety.

### BACKGROUND

Mitochondria are organelles within eukaryotic cells that produce energy (ATP) used in biological processes, and have genes that are independent of the nuclear genes. More than 80% of ATP, the energy source used by cells, is produced by mitochondria, and mutations in the mitochondrial genome (mitochondrial DNA, mtDNA) may cause fatal defects in the central nervous system, heart, muscles, visual and auditory functions, and the like. mtDNA is inherited maternally, so if there are mutations in the mother's mitochondrial DNA, they are passed on to the next generation. Most patients diagnosed with mtDNA mutations inherit the mutations from the maternal line, and approximately 40% of these are reported to arise spontaneously. Mutations in mtDNA, which cause mitochondrial diseases, occur in approximately 1 in 5,000 people. Genetic diseases caused by mutations in mitochondrial DNA are very diverse, but most of them lack effective treatments or preventive measures. Representative mitochondrial genetic disorders include Leber hereditary optic neuropathy (LHON), mitochondrial encephalopathy, lactic acidosis, and stroke-like episode (MELAS), Leigh syndrome, and the like.

Among the various mitochondrial disorders, LHON is the first genetic disease identified as being caused by mutations in mitochondrial genes, and was first described in 1871 by German ophthalmologist Theodore Leber. LHON is also called Leber's optic atrophy. Unlike other diseases, LHON is characterized by its onset and rapid progression without any distinct prodromal symptoms or pain, and may occur at any age. It is known to mainly occur in men in their 20s and 30s, with the average age of onset being 20 to 30, and most patients experience complete loss of vision in both eyes simultaneously or consecutively after a few months.

LHON is a major disease, accounting for approximately 30 to 50% of idiopathic optic neuropathies that cause vision loss in both eyes. Patients with LHON have a G→A substitution at base 3460 of the ND1 gene in mtDNA, a G→A substitution at base 11778 of the ND4 gene, or a T-+C substitution at base 14484 of the ND6 gene, which causes functional impairment of complex 1, which is composed of proteins encoded by the corresponding genes, and these three point mutations account for more than 90% of LHON onset. In conclusion, LHON is known as a major cause of bilateral unexplained optic neuropathy.

Despite extensive research on the LHON genetic disorder, there is no suitable treatment, and patients remain untreated without any effective alternative treatment and eventually lose their vision. To date, the only treatment for LHON was idebenone, which was developed by Santhera Pharmaceuticals and approved under the trade name Raxone, and although it delays the progression of blindness, it has a limitation in that it is not a fundamental cure. Accordingly, the purpose of the present invention is to provide a base editor for correcting mutations in mitochondrial DNA that causes LHON, and thereby provide a method for preventing or treating LHON using the same.

### SUMMARY

This disclosure provides a base editing composition capable of correcting a mitochondrial DNA mutation in a patient with Leber hereditary optic neuropathy (LHON), including:
one or more fusion proteins, wherein each of the one or more fusion proteins independently include DNA binding protein that specifically binds to mitochondrial DNA of a patient with LHON and further include at least one of adenine deaminase and cytosine deaminase, and
wherein cytosine deaminase is present in a full-length form or in the form of two splits.

In certain embodiments, wherein, in the patient with LHON, the composition is capable of editing:
adenine (A) at position 3460 of mitochondrial ND1 DNA to guanine (G),
adenine (A) at position 11778 of mitochondrial ND4 DNA to guanine (G), or cytosine (A) at position 14484 of mitochondrial ND6 DNA to thymine (T).

In certain embodiments, wherein cytosine deaminase is apolipoprotein B editing complex (APOBEC), activation-induced deaminase (AID), tRNA-specific adenosine deaminase (TadA), or DddAₜₒₓ, or a variant thereof.

In certain embodiments, wherein cytosine interface deaminase is DddAₜₒₓ and is included in the form of a first split and a second split, and wherein one or more amino acids located on the interface between the first and second splits are substituted with other amino acids.

In certain embodiments, wherein adenine deaminase is APOBEC, AID, or TadA, or a variant thereof.

In certain embodiments, wherein adenine deaminase includes the amino acid sequence of SEQ ID NO: 1 or a conservative amino acid substitution thereof.

In certain embodiments, wherein DNA binding protein is selected from the group consisting of zinc finger protein, TALE protein, and CRISPR-associated nuclease.

In certain embodiments, wherein one DNA binding protein binds to a nucleotide sequence of 5′-CAAACTCAAACTACGAACGCACTCACAGTCACATCATAATCCTCTCTCAAGGACT TCAAAC-3′ or a portion thereof of mitochondrial ND4 DNA.

In certain embodiments, wherein one DNA binding protein binds to a nucleotide sequence of 5′-CAAACTCAAACTACGAACGCACTCACAGTCACATCATAATCCTCTCTCAAGGACT TCAAAC-3′ or a portion thereof of mitochondrial ND4 DNA.

In certain embodiments, wherein one DNA binding protein binds to a nucleotide sequence of 5′-TCGCTGTAGTATATCCAAAGACAACCACCATTCCCCCTAAATAAATTAAAAAAAC T-3′ or a portion thereof mitochondrial ND6 DNA.

In certain embodiments, wherein the composition includes two fusion proteins and is capable of editing adenine (A) at position 3460 of mitochondrial ND1 DNA to guanine (G) in a patient with LHON,
wherein each of the two fusion proteins includes DddAₜₒₓ split and TALE protein that specifically binds to mitochondrial ND1 DNA, and
wherein one of the two fusion proteins further includes TadA8e.

In certain embodiments, wherein the composition includes two fusion proteins and is capable of editing adenine (A) at position 11778 of mitochondrial ND4 DNA to guanine (G) in a patient with LHON,
wherein each of the two fusion proteins includes DddAₜₒₓ split and TALE protein or zinc finger protein that specifically binds to mitochondrial ND4 DNA, and wherein one of the two fusion proteins further includes TadA8e.

In certain embodiments, wherein the composition includes two fusion proteins and is capable of editing cytosine (A) at position 14484 of mitochondrial ND6 DNA to thymine (T) in a patient with LHON,
wherein each of the two fusion proteins includes DddAₜₒₓ split and TALE protein that specifically binds to mitochondrial ND6.

### BRIEF DESCRIPTION OF DRAWINGS

FIG. 1 shows the results of screening the editing efficiency of m.C14484T using DdCBE (DddA-derived cytosine base editor). 1397N represents the N-terminal split of G1397 DddAₜₒₓ, and 1397C represents the C-terminal split of G1397 DddAₜₒₓ. A boxed portions on the left side represents a DNA sequence recognized by a first fusion protein comprising 1397N or 1397C, and a boxed portion on the right side represents a DNA sequence recognized by a second fusion protein comprising 1397N or 1397C. The boxed “C” indicates the base at position 14484 (T14484C) to be corrected, and a degree of boldness represents the efficiency of correction from C to T. The bar graphs on the right show the frequency of 14484C→T corrections.

FIG. 2 shows the results of screening the editing efficiency of m.A3460G using TALED (TALE-linked deaminase). 1397N represents the N-terminal split of G1397 DddAₜₒₓ, and 1397C represents the C-terminal split of G1397 DddAₜₒₓ. A boxed portion on the left side represents a DNA sequence recognized by a first fusion protein comprising TALE and 1397N, or TALE, 1397C, and TadA8e, and a boxed portion on the right side represents a DNA sequence recognized by a second fusion protein comprising TALE and 1397N, or TALE, 1397C, and TadA8e. The boxed “A” indicates the base at position 3460 (G3460A) to be corrected, and the degree of boldness represents the efficiency of correction from A to G. The bar graphs on the right show the frequency of 3460A→G corrections.

FIG. 3 shows the results of screening the efficiency of m.A11778G correction using TALED and ZFD (zinc finger deaminase). 1397N represents the N-terminal split of G1397 DddAₜₒₓ, 1397C represents the C-terminal split of G1397 DddAₜₒₓ, and ZF represents a zinc finger protein. A boxed portion on the left side represents a DNA sequence recognized by a first fusion protein comprising TALE or ZF protein and 1397N, or TALE or ZF protein, 1397C and TadA8e, and the boxed portion on the right side represents a DNA sequence recognized by a second fusion protein comprising TALE and 1397N, or TALE, 1397C and TadA8e. The boxed “A” indicates the base at position 11778 (G11778A) to be corrected, and the degree of boldness represents the efficiency of correction from A to G. The bar graphs on the right show the frequency of 11778A→G corrections.

### DETAILED DESCRIPTION OF THE DRAWINGS AND THE PRESENTLY PREFERRED EMBODIMENTS

Described herein is a base editing system for correcting mutations G3460A, G11778A, or T14484C in mitochondrial DNA of a patient with Leber hereditary optic neuropathy (LHON) to a normal genotype. Specifically, described herein is a method for correcting a mutation in the mitochondrial genes of a patient with LHON to a normal genotype using a base editor that recognizes specific sites in the mitochondrial genes of the patient with LHON and has an activity of specifically correcting the adenine base at position 3460 or 11778, or the cytosine base at position 14484, by using a fusion protein or a polynucleotide encoding such a fusion protein. The base editor or polynucleotide can exert an effect of correcting DNA mutations specific to LHON in a cellular or extracellular in vitro environment and, more preferably, can be used as a gene therapy agent capable of preventing or treating the disease. Thus, also, described herein is a use of the substance in the prevention or treatment of LHON.

A base editing system described herein uses an adenine base editor capable of correcting A at position 3460 of mitochondrial DNA of a patient with LHON to G, an adenine base editor capable of correcting A at position 11778 to G, or a cytosine base editor capable of correcting C at position 14484 to T. The above base editor utilizes a combination of one or more fusion proteins or polynucleotides encoding the one or more fusion protein, wherein the one or more fusion proteins each independently comprise a DNA binding protein and further comprise a deaminase (at least one of adenine deaminase and cytosine deaminase). The DNA binding protein used in the base editing system according to described herein may be a zinc finger protein (also called “ZF”), a transcriptional activator-like effector (TALE) protein, or a CRISPR-associated nuclease, or a combination thereof, and the deaminase may be an apolipoprotein B editing complex (APOBEC), an activation induced deaminase (AID), a tRNA-specific adenosine deaminase (TadA) or a variant thereof, and DddAₜₒₓ or a variant thereof (existing in the form of full-length or two split units), or a combination thereof. The fusion protein used in the base editing system described herein may additionally include one or more of UGI (uracil glycosylase inhibitor), NES (nuclear export signal), and MTS (mitochondrial targeting sequence). The base editor described herein may have the form of a composition of one or more fusion proteins as described above or a polynucleotide encoding the one or more fusion proteins. Prior to the present invention, there was no known method for preventing or treating LHON by correcting, through base editing, point mutations in mitochondrial genes that occur in a patient with LHON to a normal genotype.

Unless defined otherwise, all technical and scientific terms used herein have the same meaning as commonly understood by one of ordinary skill in the art to which this invention belongs. In general, the terms used herein are well known and commonly used in the art.

The terms “correct,” “edit,” and “editing” as used herein are used interchangeably and refer to a method of altering a nucleic acid sequence by selective mutation of a specific genomic target. Such a specific genomic target includes, but are not limited to, a gene, a promoter, an open reading frame or any nucleic acid sequence.

The term “base editor” or “mitochondrial DNA base editor” as used herein means a substance capable of altering a nucleic acid sequence by selective mutation of a mitochondrial genome target, and includes a combination of one or more different base editors. The term “base editor” or “mitochondrial DNA base editor” as used herein may be in the form of a polypeptide (which may be a fusion protein) or a polynucleotide, depending on the context, and may be a composition comprising one or more polypeptides (which may be fusion proteins) or polynucleotides. That is, the term “base editing composition” as used herein means a combination of one or more different base editors, wherein the different base editors may be used simultaneously or separately.

The term “target” or “target site” as used herein means a pre-identified nucleic acid sequence of any composition and/or length. Such a target site includes, but are not limited to, a gene, a promoter, an open reading frame or any nucleic acid sequence.

In one embodiment, described herein is a base editor capable of editing a mitochondrial DNA mutation of a patient with Leber hereditary optic neuropathy (LHON), wherein the base editor comprises one or more fusion proteins, wherein the one or more fusion proteins each independently comprise a DNA binding protein that specifically binds to the mitochondrial DNA of the patient with LHON, and may have a form of a composition additionally comprising one or more of adenine deaminase and cytosine deaminase. The cytosine deaminase may be present in a full-length form or in the form of two splits.

The base editor descried herein is capable of editing adenine (A) at position 3460 of mitochondrial ND1 DNA of a patient with LHON to guanine (G), adenine (A) at position 11778 of mitochondrial ND4 DNA to guanine (G), or cytosine (C) at position 14484 of mitochondrial ND6 DNA to thymine (T).

In this specification, a person skilled in the art would understand that the base at position 3460 of ND1 DNA is referred to the 3460th base among all bases constituting the mitochondrial DNA and indicates a base constituting the ND1 DNA, and the base at position 11778 of ND4 DNA is referred to the 11778th base among all bases constituting the mitochondrial DNA and indicates a base constituting the ND4 DNA, and the base at position 14484 of ND6 DNA is referred to the 14484th base among all bases constituting the mitochondrial DNA and indicates a base constituting the ND6 DNA.

The cytosine deaminase that may be used in the base editor described herein means an amino group deaminase capable of converting a cytosine into uridine, and may be derived from or mutated (e.g., engineered or evolved) from any organism (e.g., eukaryotes or prokaryotes) including, but not limited to, algae, bacteria, fungi, plants, invertebrates, and mammals. For example, it may be a cytosine deaminase derived from or mutated from APOBEC (apolipoprotein B editing complex), AID (activation-induced deaminase), TadA (tRNA-specific adenosine deaminase), a bacterial adenine deaminase, or an ortholog thereof, or a cytosine deaminase derived from or mutated by DddA, a bacterial cytosine deaminase, or an ortholog thereof, or a fragment thereof. The cytosine deaminase mutated from the above-mentioned TadA may be, for example, one in which one or more of the amino acid residues 6, 26, 27, 28, 46, 48, 49, 61, 74, 76, 77, 82, 96, 107, 108, 112, 114, 115, 119, 122, 127, 142, 143, 151, 154 and 158 of the amino acid sequence of SEQ ID NO: 1 are mutated to another amino acid. For example, it may be a polypeptide in which the 27th amino acid in the amino acid sequence of SEQ ID NO: 1 is mutated to lysine, the 28th amino acid is mutated to alanine, the 61st amino acid is mutated to isoleucine, and the 96th amino acid is mutated to asparagine. Regarding the composition of the cytosine deaminase that may be used here, reference is made to international patent application publications nos. WO 2022/060185 and WO 2023/086953, and the like, which are incorporated by reference in their entirety into this application.

When the base editor described herein includes cytosine deaminase, the cytosine deaminase may be included in the form of a first split and a second split, and may also be included in a full-length form. In the case where the first split and the second split are provided, the first split and the second split may each be in a form linked to a DNA binding protein.

In this specification, when two proteins are said to be “linked,” the two proteins may be directly linked or indirectly linked via a linker or other protein(s).

The cytosine deaminase as used herein may be DddAₜₒₓ, which is a portion of a bacterial toxin derived from Burkholderia cenocepacia that exhibits an enzymatic function and may deaminate cytosine of double-stranded DNA. DddAₜₒₓ may comprise the amino acid sequence of SEQ ID NO: 2.

SEQ ID NO: 2: wild-type DddAₜₒₓ
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVKRGATGETKVFTGNSNSPKSPTK
GGC

Since DddAₜₒₓ is toxic to cells, it may be used in the form of two inactive splits, namely a first split and a second split, to avoid toxicity in a host cell. When the cytosine deaminase as used herein is used in the form of a first split and a second split, each of the first split and the second split has no deamination activity.

The first split of the DddAₜₒₓ cytosine deaminase may comprise a sequence from the N-terminus to G33, G44, A54, N68, G82, N98, or G108 of the amino acid sequence of SEQ ID NO: 2, and the second split may comprise a sequence from G34, P45, G55, N69, T83, A99, or A109 of the amino acid sequence of SEQ ID NO: 2 to the C-terminus.

Preferably, the first split of the DddAₜₒₓ cytosine deaminase may comprise a sequence from the N-terminus to G44 of the amino acid sequence of SEQ ID NO: 2 (SEQ ID NO: 3 below), and the second split may comprise a sequence from P45 to the C-terminus (SEQ ID NO: 4 below).

SEQ ID NO: 3:
wild-type DddAₜₒₓ G1333-N
GSYALGPYQISAPQLPAYNGQTVGTFYYVNDAGGLESKVFSSGG
SEQ ID NO: 4:
wild-type DddAₜₒₓ G1333-C
PTPYPNYANAGHVEGQSALFMRDNGISEGLVFHNNPEGTCGFCVN
MTETLLPENAKMTVVPPEGAIPVKRGATGETKVFTGNSNSPKSPT
KGGC

Preferably, the first split of the DddAₜₒₓ cytosine deaminase may comprise the sequence from the N-terminus to G108 of the amino acid sequence of SEQ ID NO: 2 (SEQ ID NO: 5 below), and the second split may comprise the sequence from A109 to the C-terminus (SEQ ID NO: 6 below).

SEQ ID NO: 5:
wild-type DddAₜₒₓ G1397-N
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEG
SEQ ID NO: 6:
wild-type DddAₜₒₓ G1397-C
AIPVKRGATGETKVFTGNSNSPKSPTKGGC

When the first split and the second split of DddAₜₒₓ are used as cytosine deaminase, one or more amino acids located at the surface where the first split and the second split of the cytosine deaminase bind to each other may be substituted with other amino acids. For example, the first split and the second split of DddAₜₒₓ may each comprise the amino acid sequences of SEQ ID NO: 3 (G1333-N) and SEQ ID NO: 4 (G1333-C), in which case, at least one amino acid selected from the group consisting of positions 3, 5, 10, 11, 13, 14, 15, 16, 17, 18, 19, 28, 30 and 31 of SEQ ID NO: 3 or at least one amino acid selected from the group consisting of positions 13, 16, 17, 20, 21, 28, 29, 30, 31, 32, 33, 56, 57, 58 and 60 of SEQ ID NO: 4 may be substituted with another amino acid, but is not limited thereto. In another example, the first split of DddAₜₒₓ may comprise the amino acid sequence of SEQ ID NO: 5 (G1397-N) and SEQ ID NO: 6 (G1397-C), wherein at least one amino acid selected from the group consisting of positions 87, 88, 91, 92, 95, 100, 101, 102 and 103 of SEQ ID NO: 5 or at least one amino acid selected from the group consisting of positions 13, 14, 15 and 16 of SEQ ID NO: 6 may be substituted with another amino acid, but is not limited thereto. The term “another amino acid” refers to an amino acid selected from among alanine, isoleucine, leucine, methionine, phenylalanine, proline, tryptophan, valine, aspartic acid, cysteine, glutamine, glycine, serine, threonine, tyrosine, aspartic acid, glutamic acid, arginine, histidine, lysine, and all known variants of the above amino acids, excluding the amino acid that the wild-type protein originally has at the mutation position. Using such a variant, when the pair of DddAₜₒₓ splits, each linked to a DNA-binding protein, fails to bind to DNA, the pair does not function properly, thereby causing highly efficient and precise C-to-T correction without undesired off-target C-to-T correction. As examples of the variant, there may be provided a first split of DddAₜₒₓ having the amino acid sequence of SEQ ID NO: 139 (which may be referred to as “G1397-N” or “G1397N”) and a second split of DddAₜₒₓ having the amino acid sequence of SEQ ID NO: 140 (which may be referred to as “G1397-C” or “G1397C”).

The terms “G1333-N”, “G1333N” or “1333N” may refer to a first split of wild-type DddAₜₒₓ having an amino acid sequence of SEQ ID NO: 3, or an amino acid variant thereof, and the terms “G1333-C”, “G1333C” or “1333C” may refer to a second split of wild-type DddAₜₒₓ having an amino acid sequence of SEQ ID NO: 4, or an amino acid variant thereof.

The terms “G1397-N”, “G1397N” or “1397N” may refer to a first split of wild-type DddAₜₒₓ having an amino acid sequence of SEQ ID NO: 5 or 139, or an amino acid variant thereof, and the terms “G1397-C”, “G1397C” or “1397C” may refer to a second split of wild-type DddAₜₒₓ having an amino acid sequence of SEQ ID NO: 6 or 140, or an amino acid variant thereof.

The cytosine deaminase as used herein may be used in a full-length form, and the full-length cytosine deaminase (e.g., DddAₜₒₓ) used in this case has an amino acid sequence that is modified to reduce or eliminate toxicity. The C-terminus of DddAₜₒₓ is specifically enriched with positively charged amino acids. Because DNA is negatively charged, it binds to positively charged amino acids in proteins. By substituting this positively charged amino acid, the binding strength of DddAₜₒₓ to DNA may be weakened, thereby reducing or eliminating intracellular toxicity. That is, if a positively charged amino acid is substituted to eliminate the toxicity, cloning using E. coli is possible, thereby securing full-length DddAₜₒₓ. Such non-toxic full-length cytosine deaminase may be provided by substituting one or more, two or more, three or more, four or more, or five or more amino acids of the wild-type amino acid sequence of SEQ ID NO: 2 with another amino acid. The “another amino acid” refers to an amino acid selected from among alanine, isoleucine, leucine, methionine, phenylalanine, proline, tryptophan, valine, aspartic acid, cysteine, glutamine, glycine, serine, threonine, tyrosine, aspartic acid, glutamic acid, arginine, histidine, lysine, and all known variants of the above amino acids, excluding the amino acid that the wild-type protein originally has at the mutation position. For example, the another amino acid may be alanine.

The non-toxic full-length DddAₜₒₓ may comprise an amino acid sequence selected from the group consisting of the following amino acid sequences.

A1341D KRKKA Variant
GSYALGPYQISAPQLPAYNGQTVGTFYYVNDAGGLESKVFSSGGP
TPYPNYDNAGHVEGQSALFMRDNGISEGLVFHNNPEGTCGFCVNM
TETLLPENAKMTVVPPEGAIPVKRGATGETKVFTGNSNSPKSPTA
GGC
AAAAA Variant
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVAAGATGETAVFTGNSNSPASPTA
GGC
AAAAK Variant
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVAAGATGETAVFTGNSNSPASPTK
GGC
AAKAA Variant
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVAAGATGETKVFTGNSNSPASPTA
GGC
AAKAK Variant
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVAAGATGETKVFTGNSNSPASPTK
GGC
KAAAA Variant
GSYALGPYQISAPQLPAYNGQTVGTFYYVNDAGGLESKVFSSGGP
TPYPNYANAGHVEGQSALFMRDNGISEGLVFHNNPEGTCGFCVNM
TETLLPENAKMTVVPPEGAIPVKAGATGETAVFTGNSNSPASPTA
GGC
E1347A Variant
GSYALGPYQISAPQLPAYNGQTVGTFYYVNDAGGLESKVFSSGGP
TPYPNYANAGHVAGQSALFMRDNGISEGLVFHNNPEGTCGFCVNM
TETLLPENAKMTVVPPEGAIPVKRGATGETKVFTGNSNSPKSPTK
GGC

Preferably, the full-length cytosine deaminase variant that may be used herein may have one or more amino acid substitutions selected from the group consisting of a substitution of S at position 37 to G, a substitution of G at position 59 to S, a substitution of A at position 109 to V, and a substitution of S at position 129 to G in the amino acid sequence of SEQ ID NO: 2.

More preferably, the full-length cytosine deaminase variant that may be used herein may have all of the substitution of S at position 37 to G, the substitution of G at position 59 to S, the substitution of A at position 109 to V and the substitution of S at position 129 to G in the amino acid sequence of SEQ ID NO: 2, in which case the sequence is as follows.

GSVG Variant
GSYALGPYQISAPQLPAYNGQTVGTFYYVNDAGGLEGKVFSSGGP
TPYPNYANAGHVESQSALFMRDNGISEGLVFHNNPEGTCGFCVNM
TETLLPENAKMTVVPPEGVIPVKRGATGETKVFTGNSNGPKSPTK
GGC

In another example, the full-length cytosine deaminase variant that may be used in the present invention may comprise the following sequences.

SSVG Variant
GSYALGPYQISAPQLPAYNGQTVGTFYYVNDAGGLESKVFSSGGP
TPYPNYANAGHVESQSALFMRDNGISEGLVFHNNPEGTCGFCVNM
TETLLPENAKMTVVPPEGVIPVKRGATGETKVFTGNSNGPKSPTK
GGC
GSAG Variant
GSYALGPYQISAPQLPAYNGQTVGTFYYVNDAGGLEGKVFSSGGP
TPYPNYANAGHVESQSALFMRDNGISEGLVFHNNPEGTCGFCVNM
TETLLPENAKMTVVPPEGAIPVKRGATGETKVFTGNSNGPKSPTK
GGC
GSVS Variant
GSYALGPYQISAPQLPAYNGQTVGTFYYVNDAGGLEGKVFSSGGP
TPYPNYANAGHVESQSALFMRDNGISEGLVFHNNPEGTCGFCVNM
TETLLPENAKMTVVPPEGVIPVKRGATGETKVFTGNSNSPKSPTK
GGC

An adenine deaminase that may be used in the base editor described herein means an amino group deaminase capable of converting an adenine base into inosine, and may be derived from or mutated (e.g., engineered or evolved) from any organism (e.g., a eukaryote or prokaryote), including but not limited to algae, bacteria, fungi, plants, invertebrates, and mammals, for example, E. coli, S. aureus, S. typhi, S. putrefaciens, H. influenzae, or C. crescentus. Such adenine deaminase may be, for example, APOBEC, AID, or TadA, or a variant thereof. The aforementioned TadA may be, for example, TadA8e (SEQ ID NO: 1) or a truncated form or a variant thereof (for example, a variant improved or evolved to be applicable to deoxynucleotides). The above-mentioned variant of TadA8e may be, for example, one in which one or more of amino acid residues 23, 28, 30, 36, 46, 48, 49, 51, 76, 82, 82, 84, 106, 108, 110, 111, 146, 147, 152, 154, 155, 156 and 157 of SEQ ID NO: 1 are mutated to another amino acid. Regarding the composition of adenine deaminase that may be used herein, reference may be made to international patent application publications nos. WO 2022/060185, WO 2023/086953, and the like, which are incorporated by reference in their entirety into this application. In certain embodiments, an adenine deaminase that may be used herein may comprise the amino acid sequence of SEQ ID NO: 1, or a conservative amino acid substitution thereof.

The DNA binding protein used in the base editor described herein may be a zinc finger protein, a TALE protein, a CRISPR-associated nuclease, or a combination thereof. With respect to the compositions of the zinc finger protein, the TALE protein, and the CRISPR-associated nuclease, reference may be made to International Patent Application Publication No. WO2022/060185, which is incorporated by reference in its entirety into this application.

The zinc finger is a representative DNA-binding protein structure that forms a major DNA-binding protein motif, and the interaction between the α-helix of the zinc finger and the major groove of DNA enables strong and specific recognition of DNA sequences. When one or more zinc finger motifs are used in combination and a DNA binding protein used in a base editor described herein is a zinc finger protein, amino acid sequences of SEQ ID NOs: 7 to 9 may be included.

The DNA binding protein used in the base editor described herein may be a “TALE protein.” The TALE protein refers to a protein that binds to nucleotides in a sequence-specific manner via one or more TALE-repeat modules. The TALE protein comprises at least one TALE-repeat module, preferably, but not limited to, 1 to 30 TALE-repeat modules. As used herein, the “TALE-repeat modules” may be referred to as a “TALE array”, and the term “TALE protein” refers to a configuration comprising an N-terminal domain and a C-terminal domain (which may comprise a half domain) on each side of the TALE array. The term “TALE” as used herein may mean only “TALE array” or “TALE protein” depending on the context. When the DNA binding protein used in the base editor according to the present invention is a TALE protein, amino acid sequences of SEQ ID NOs: 10 to 65 may be included.

In certain embodiments, when a TALE protein is used as a DNA binding protein of a base editor used herein, a single module TALE array or a multi-module TALE array (e.g., a dual module TALE array comprising a first TALE (or left TALE) array and a second TALE (or right TALE) array) may be used.

In certain embodiments, when the base editor used herein comprises two fusion proteins, each of which comprises a TALE protein, the two fusion proteins respectively have a first TALE protein (or left TALE) and a second TALE protein (or right TALE). The first TALE protein and the second TALE protein may each independently be linked, directly or indirectly (e.g., via a linker and/or other protein component), to at least one of a cytosine deaminase and an adenine deaminase. For example, a first fusion protein (a fusion protein that binds DNA 5′ upstream from the base-editing target site) comprising a first TALE protein (left TALE) may comprise a first split of cytosine deaminase, a second fusion protein comprising a second TALE protein (right TALE) may comprise a second split of cytosine deaminase, and either or both of the first fusion protein and the second fusion protein may comprise an adenine deaminase. Alternatively, a first fusion protein comprising a first TALE protein (left TALE) may comprise a second split of cytosine deaminase, a second fusion protein comprising a second TALE protein (right TALE) may comprise a first split of cytosine deaminase, and either or both of the first fusion protein and the second fusion protein may comprise an adenine deaminase. Alternatively, the full-length form of cytosine deaminase may be included in either a first fusion protein comprising a first TALE protein or a second fusion protein comprising a second TALE protein, and either or both of the first fusion protein and the second fusion protein may comprise an adenine deaminase.

In certain embodiments, when the base editor described herein comprises two fusion proteins, one of the two fusion proteins may comprise a TALE protein and the other may comprise a zinc finger protein. For example, a first fusion protein (a fusion protein that binds DNA 5′ upstream from a base-editing target site) may comprise a TALE protein (left TALE), and a second fusion protein (a fusion protein that binds DNA 3′ downstream from the base-editing target site) may comprise a zinc finger protein (right ZF). Alternatively, a first fusion protein (a fusion protein that binds DNA 5′ upstream from a base-editing target site) may comprise a zinc finger protein (left ZF), and a second fusion protein (a fusion protein that binds DNA 3′ downstream from the base-editing target site) may comprise a TALE protein (right TALE). In both of the above two methods, either or both of the first fusion protein and the second fusion protein may comprise an adenine deaminase.

In certain embodiments, when a cytosine deaminase is included in a fusion protein comprising a TALE protein or a zinc finger protein, the cytosine deaminase may be linked directly or indirectly (e.g., via a linker and/or other protein component) to the N-terminus or C-terminus (preferably the C-terminus) of the TALE protein, or to the N-terminus or C-terminus (preferably the N-terminus) of the zinc finger protein. When an adenine deaminase is included in a fusion protein comprising a TALE protein or a zinc finger protein, the adenine deaminase may be directly or indirectly linked (e.g., via a linker and/or other protein component) to the N-terminus or C-terminus (preferably the C-terminus) of the TALE protein or to the N-terminus or C-terminus (preferably the N-terminus) of the zinc finger protein. When both of the a cytosine deaminase and an adenine deaminase are included in a fusion protein comprising a TALE protein or a zinc finger protein, the adenine deaminase may be linked directly or indirectly (e.g., via a linker and/or other protein component) to the N-terminus or C-terminus (preferably the C-terminus) of the cytosine deaminase.

In certain embodiments, when the base editor described herein comprises two fusion proteins, the fusion proteins may have different combinations and arrangements of protein components (e.g., DNA binding protein, cytosine deaminase, and adenine deaminase). For example, while a first fusion protein (a fusion protein that binds DNA 5′ upstream from a base-editing target site) may comprise an adenine deaminase, a second fusion protein (a fusion protein that binds DNA 3′ downstream from the base-editing target site) may not comprise an adenine deaminase, or vice versa. For example, while a first fusion protein (a fusion protein that binds DNA 5′ upstream from a base-editing target site) may comprise a TALE protein, a second fusion protein (a fusion protein that binds DNA 3′ downstream from the base-editing target site) may comprise a zinc finger protein, or vice versa.

In certain embodiments, when it comes to the arrangement of protein components of the fusion proteins, a first fusion protein (a fusion protein that binds to DNA 5′ upstream from the base-editing target site) and a second fusion protein (a fusion protein that binds to DNA 3′ downstream from the base-editing target site) may have independently arranged protein components. For example, in the first fusion protein, the adenine deaminase may be positioned after the C-terminus of the cytosine deaminase, whereas in the second fusion protein, the adenine deaminase may be positioned before the N-terminus of the cytosine deaminase, or vice versa. For example, in the first fusion protein, the DNA binding protein may be positioned after the C-terminus of the cytosine deaminase, whereas in the second fusion protein, the DNA binding protein may be positioned before the N-terminus of the cytosine deaminase, or vice versa.

One or more fusion proteins included in the base editor described herein may each independently, additionally include a UGI (uracil glycosylase inhibitor). UGI may increase base editing efficiency by inhibiting the activity of UDG (uracil DNA glycosylase), which is an enzyme that repairs mutated DNA by catalyzing the removal of U from DNA. When a UGI is used in the base editor according to the present invention, the location of the UGI may vary, and for example, the UGI may be directly or indirectly linked (for example, via a linker and/or other protein components) to the C-terminus of cytosine deaminase, but is not limited thereto. When UGI is used in the base editor according to the present invention, the UGI may comprise the amino acid sequence of SEQ ID NO: 126.

One or more fusion proteins included in the base editor described herein may additionally include a nuclear export signal (NES). Attaching an NES to a base-editing protein may result in higher efficiency of base editing. The NES sequence may be any signal sequence (e.g., SEQ ID NO: 127) that confers the ability to translocate outside the nucleus, and a natural NES or an artificially synthesized NES may be used. For example, it may be derived from mirute virus of mice (MVM), but is not limited to. When an NES is used in the base editor described herein, the location of the NES may vary and may be, for example, directly or indirectly linked to the N-terminus of cytosine deaminase (e.g., via a linker and/or other protein components), but is not limited to. When an NES is used in the base editor described herein, the NES may comprise the amino acid sequence of SEQ ID NO: 127.

In certain embodiments, the base editor (fusion protein) described herein may additionally include an MTS (mitochondrial targeting sequence). The MTS may be any signal sequence capable of translocating into mitochondria, and may be a natural MTS present at the N-terminus of various mitochondrial proteins, or an artificially synthesized MTS may also be used. When MTS is used in the base editor described herein, the location of the MTS may vary, and for example, the MTS may be directly or indirectly linked (for example, via a linker and/or other protein components) to the N-terminus of a DNA binding protein or the N-terminus of an NES, but is not limited thereto. In certain embodiments, when MTS is used in the base editor described herein, the MTS may comprise any one of the amino acid sequences of SEQ ID NOs: 128 to 130.

The DNA binding protein used in the base editor described herein, in whole or in part, may recognize and bind to the nucleotide sequence of 5′-TACGGGCTACTACAACCCTTCGCTGACACCATAAAACTCTTCACCAAAGAGCCCCT AAA-3′ (the underlined and bold A is the base at position 3460) or a portion thereof of the mitochondrial ND1 DNA sequence. 5′-Preferably, it may recognize TACGGGCTACTACAACCCTTCG-3′ or a portion thereof, and/or 5′-TAAAACTCTTCACCAAAGAGCCCCTAAA-3′ or a portion thereof of the mitochondrial ND1 DNA sequence. In certain embodiments, when the base editor described herein is in the form of a composition of one or more different base editors, one base editor (the first fusion protein) may recognize 5′-TACGGGCTACTACAACCCTTCG-3′ or a portion thereof of the mitochondrial DNA sequence, and another base editor (the second fusion protein) may recognize 5′-TAAAACTCTTCACCAAAGAGCCCCTAAA-3′ or a portion thereof of the mitochondrial DNA sequence.

The DNA binding protein used in the base editor described herein, in whole or in part, may recognize and bind to the nucleotide sequence of 5′-CAAACTCAAACTACGAACGCACTCACAGTCACATCATAATCCTCTCTCAAGGACTT CAAAC-3′ (the underlined and bold A is the base at position 11778) or a portion thereof of the mitochondrial ND4 DNA sequence. Preferably, it may recognize 5′-CAAACTCAAACTACGAACGCACTCACAGTC-3′ or a portion thereof, and/or 5′-CATAATCCTCTCTCAAGGACTTCAAAC-3′ or a portion thereof of the mitochondrial ND4 DNA sequence. In certain embodiments, when the base editor described herein is in the form of a composition of one or more different base editors, one base editor (the first fusion protein) may recognize 5′-CAAACTCAAACTACGAACGCACTCACAGTC-3′ or a portion thereof of the mitochondrial DNA sequence, and another base editor (the second fusion protein) may recognize 5′-CATAATCCTCTCTCAAGGACTTCAAAC-3′ or a portion thereof of the mitochondrial DNA sequence.

The DNA binding protein used in the base editor described herein, in whole or in part, may recognize 5′-TAGCCATCGCTGTAGTATATCCAAAGACAACCACCATTCCCCCTAAATAAATTAAAA AAACTA-3′ (the underlined and bold C is the base at position 14484) in a mitochondrial ND6 DNA thereof, 5′-sequence or a portion preferably TCGCTGTAGTATATCCAAAGACAACCACCATTCCCCCTAAATAAATTAAAAAAACT A-3′ or a portion thereof. Preferably, it may recognize 5′-TCGCTGTAGTATATCCAAAGACA-3′ or a portion thereof, and/or 5′-TCCCCCTAAATAAATTAAAAA-3′ or a portion thereof of the mitochondrial DNA sequence. In certain embodiments, when the base editor described herein is in the form of a composition of one or more different base editors, one base editor (the first fusion protein) may recognize 5′-TCGCTGTAGTATATCCAAAGACA-3′ or a portion thereof of the mitochondrial DNA sequence, and another base editor (the second fusion protein) may recognize 5′-TCCCCCTAAATAAATTAAAAA-3′ or a portion thereof of the mitochondrial DNA sequence.

The DNA binding protein used in the base editor described herein may comprise any one of the amino acid sequences of SEQ ID NOs: 7 to 65, or a conservative substitution thereof.

A “conservative amino acid substitution” refers to the substitution of an amino acid residue with another residue having a side chain (R group) with similar chemical properties (e.g., charge or hydrophobicity). In general, conservative amino acid substitutions do not substantially change the functional properties of a protein. When two or more amino acid sequences differ from each other by conservative substitutions, the % sequence identity or similarity may be adjusted upward to compensate for the conservative nature of the substitutions. Means for performing this adjustment are well known to those skilled in the art [see, e.g., Pearson (1994) Methods Mol. Biol. 24:307-31]. Examples of amino acid groups having side chains with similar chemical properties include: (1) an aliphatic side chain: glycine, alanine, valine, leucine, and isoleucine; (2) an aliphatic-hydroxyl side chain: serine and threonine; (3) an amide-containing side chain: asparagine and glutamine; (4) an aromatic side chain: phenylalanine, tyrosine, and tryptophan; (5) a basic side chain: lysine, arginine, and histidine; (6) an acidic side chain: aspartate and glutamate; and (7) a sulfur-containing side chain: cysteine and methionine. Preferred conservative amino acid substitution groups are: valine-leucine-isoleucine, phenylalanine-tyrosine, lysine-arginine, alanine-valine, glutamate-aspartate, and asparagine-glutamine. Alternatively, a conservative substitution may be any change having a positive value in the PAM250 log-odds matrix described in Gonnet et al. (1992) Science 256:1443-1445.

The base editing composition described herein is capable of editing adenine (A) at position 3460 of mitochondrial ND1 DNA to guanine (G) in a patient with LHON, and comprises two fusion proteins, wherein the two fusion proteins each comprise a TALE protein and a DddAₜₒₓ split that specifically bind to mitochondrial ND1 DNA, and one of the two fusion proteins may additionally comprise TadA8e.

In the base editing composition, one fusion protein may comprise a TALE protein (left TALE) comprising an amino acid sequence selected from the group consisting of SEQ ID NOs: 10 to 17 or a conservative amino acid substitution thereof, and the other fusion protein may comprise a TALE protein (right TALE) comprising an amino acid sequence selected from the group consisting of SEQ ID NOs: 18 to 31 or a conservative amino acid substitution thereof.

The base editing composition described herein is capable of editing adenine (A) at position 11778 of mitochondrial ND4 DNA to guanine (G) in a patient with LHON, and comprises two fusion proteins, wherein the two fusion proteins each comprise a TALE or zinc finger protein and a DddAₜₒₓ split that specifically bind to mitochondrial ND4 DNA, and one of the two fusion proteins may further comprise TadA8e.

In the base editing composition, one fusion protein may comprise a TALE protein (left TALE) comprising an amino acid sequence selected from the group consisting of SEQ ID NOs: 32 to 42 or a conservative amino acid substitution thereof, or a zinc finger protein (left ZF) comprising an amino acid sequence selected from the group consisting of SEQ ID NOs: 7 to 9 or a conservative amino acid substitution thereof, and the other fusion protein may comprise a TALE protein (right TALE) comprising an amino acid sequence selected from the group consisting of SEQ ID NOs: 43 to 53 or a conservative amino acid substitution thereof, or a zinc finger protein (right ZF) comprising an amino acid sequence selected from the group consisting of SEQ ID NOs: 7 to 9 or a conservative amino acid substitution thereof.

The base editing composition described herein is capable of editing cytosine (A) at position 14484 of mitochondrial ND6 DNA to thymine (T) in a patient with LHON, and comprises two fusion proteins, wherein the two fusion proteins may each include a TALE protein and a DddAₜₒₓ split that specifically bind to mitochondrial ND6 DNA.

In the base editing composition, one fusion protein may comprise a TALE protein (left TALE) comprising an amino acid sequence selected from the group consisting of SEQ ID NOs: 54 to 57 or a conservative amino acid substitution thereof, and the other fusion protein may comprise a TALE protein (right TALE) comprising an amino acid sequence selected from the group consisting of SEQ ID NOs: 58 to 65 or a conservative amino acid substitution thereof.

The term “fusion protein” as used herein refers to a polypeptide formed by binding two or more different polypeptides via peptide bonds. The fusion protein is capable of editing adenine (A) at position 3460 of mitochondrial DNA in a patient with LHON to guanine (G), adenine (A) at position 11778 to guanine (G), or cytosine (A) at position 14484 to thymine (T), and comprises a DNA binding protein, additionally comprises a deaminase (at least one of adenine deaminase and cytosine deaminase), and may further comprise UGI, NES, and/or MTS. A method for designing and constructing a fusion protein (or a polynucleotide encoding a fusion protein) may be any method known in the art, and the polynucleotide may be inserted into a vector, and the vector may be introduced into a cell. Individual proteins constituting a fusion protein described herein are typically cloned into a single polynucleotide and expressed as a single polypeptide (fusion protein), but one or more of the individual proteins may be cloned into separate polynucleotides and expressed as two or more separate polypeptides, and such a case also falls within the scope of the present invention.

In certain embodiments, a linker that may be used in a fusion protein described herein may be a peptide linker comprising 2 to 40 amino acid residues. The length may be, for example, a length of 2, 5, 10, 16, 24, or 32 amino acids, but is not limited thereto. Linkers used herein may comprise, for example, the following linkers.

GS
(SEQ ID NO: 131)
SGSETPGTSESATPES
(SEQ ID NO: 132)
SGTPHEVGVYTLSGTPHEVGVYTL
(SEQ ID NO: 133)
AAEFGIHGVPAAMG
(SEQ ID NO: 134)
AAEFGIHGVPAAMGGS
(SEQ ID NO: 135)
SGGS

In certain embodiments, described herein is a polynucleotide encoding any one of one or more fusion proteins included in the base editing composition. A base editor described herein may comprise a polynucleotide encoding the fusion protein described above.

The base editor described herein may be in the form of a composition of different polynucleotides encoding different base editors.

### Composition Examples Of Base Editor (Or Base Editing Composition)

The base editor (or base editing composition) described herein may comprise a combination of a fusion protein having an amino acid sequence selected from the group consisting of SEQ ID NOs: 78 to 85 and a fusion protein having an amino acid sequence selected from the group consisting of SEQ ID NOs: 86 to 99.

For example, a base editor (or base editing composition) may comprise a combination of fusion proteins selected from the group consisting of the following pairs of fusion proteins:
A fusion protein having an amino acid sequence of SEQ ID NO: 78 and a fusion protein having an amino acid sequence of SEQ ID NO: 90;
A fusion protein having an amino acid sequence of SEQ ID NO: 78 and a fusion protein having an amino acid sequence of SEQ ID NO: 91;
A fusion protein having an amino acid sequence of SEQ ID NO: 79 and a fusion protein having an amino acid sequence of SEQ ID NO: 92;
A fusion protein having an amino acid sequence of SEQ ID NO: 79 and a fusion protein having an amino acid sequence of SEQ ID NO: 93;
A fusion protein having an amino acid sequence of SEQ ID NO: 79 and a fusion protein having an amino acid sequence of SEQ ID NO: 94;
A fusion protein having an amino acid sequence of SEQ ID NO: 79 and a fusion protein having an amino acid sequence of SEQ ID NO: 90;
fusion protein having an amino acid sequence of SEQ ID NO: 79 and a fusion protein having an amino acid sequence of SEQ ID NO: 91;
A fusion protein having an amino acid sequence of SEQ ID NO: 80 and a fusion protein having an amino acid sequence of SEQ ID NO: 95;
A fusion protein having an amino acid sequence of SEQ ID NO: 80 and a fusion protein having an amino acid sequence of SEQ ID NO: 90;
A fusion protein having an amino acid sequence of SEQ ID NO: 80 and a fusion protein having an amino acid sequence of SEQ ID NO: 91;
A fusion protein having an amino acid sequence of SEQ ID NO: 81 and a fusion protein having an amino acid sequence of SEQ ID NO: 96;
A fusion protein having an amino acid sequence of SEQ ID NO: 81 and a fusion protein having an amino acid sequence of SEQ ID NO: 91;
A fusion protein having an amino acid sequence of SEQ ID NO: 82 and a fusion protein having an amino acid sequence of SEQ ID NO: 97;
A fusion protein having an amino acid sequence of SEQ ID NO: 82 and a fusion protein having an amino acid sequence of SEQ ID NO: 90;
A fusion protein having an amino acid sequence of SEQ ID NO: 83 and a fusion protein having an amino acid sequence of SEQ ID NO: 88;
A fusion protein having an amino acid sequence of SEQ ID NO: 83 and a fusion protein having an amino acid sequence of SEQ ID NO: 87;
A fusion protein having an amino acid sequence of SEQ ID NO: 83 and a fusion protein having an amino acid sequence of SEQ ID NO: 89;
A fusion protein having an amino acid sequence of SEQ ID NO: 84 and a fusion protein having an amino acid sequence of SEQ ID NO: 88;
A fusion protein having an amino acid sequence of SEQ ID NO: 84 and a fusion protein having an amino acid sequence of SEQ ID NO: 86;
A fusion protein having an amino acid sequence of SEQ ID NO: 84 and a fusion protein having an amino acid sequence of SEQ ID NO: 87; and
A fusion protein having an amino acid sequence of SEQ ID NO: 85 and a fusion protein having an amino acid sequence of SEQ ID NO: 88.

In certain embodiments, the base editor (or base editing composition) described herein may comprise a combination of a fusion protein comprising an amino acid sequence selected from the group consisting of SEQ ID NOs: 100 to 114 and a fusion protein comprising an amino acid sequence selected from the group consisting of SEQ ID NOs: 115 to 125.

For example, a base editor (or base editing composition) may comprise a combination of fusion proteins selected from the group consisting of the following pairs of fusion proteins:
A fusion protein having an amino acid sequence of SEQ ID NO: 100 and a fusion protein having an amino acid sequence of SEQ ID NO: 118;
A fusion protein having an amino acid sequence of SEQ ID NO: 101 and a fusion protein having an amino acid sequence of SEQ ID NO: 119;
A fusion protein having an amino acid sequence of SEQ ID NO: 101 and a fusion protein having an amino acid sequence of SEQ ID NO: 118;
A fusion protein having an amino acid sequence of SEQ ID NO: 102 and a fusion protein having an amino acid sequence of SEQ ID NO: 118;
fusion protein having an amino acid sequence of SEQ ID NO: 103 and a fusion protein having an amino acid sequence of SEQ ID NO: 120;
A fusion protein having an amino acid sequence of SEQ ID NO: 103 and a fusion protein having an amino acid sequence of SEQ ID NO: 119;
A fusion protein having an amino acid sequence of SEQ ID NO: 103 and a fusion protein having an amino acid sequence of SEQ ID NO: 118;
A fusion protein having an amino acid sequence of SEQ ID NO: 113 and a fusion protein having an amino acid sequence of SEQ ID NO: 118;
A fusion protein having an amino acid sequence of SEQ ID NO: 104 and a fusion protein having an amino acid sequence of SEQ ID NO: 119;
A fusion protein having an amino acid sequence of SEQ ID NO: 104 and a fusion protein having an amino acid sequence of SEQ ID NO: 118;
A fusion protein having an amino acid sequence of SEQ ID NO: 104 and a fusion protein having an amino acid sequence of SEQ ID NO: 121;
A fusion protein having an amino acid sequence of SEQ ID NO: 104 and a fusion protein having an amino acid sequence of SEQ ID NO: 122;
A fusion protein having an amino acid sequence of SEQ ID NO: 105 and a fusion protein having an amino acid sequence of SEQ ID NO: 120;
A fusion protein having an amino acid sequence of SEQ ID NO: 105 and a fusion protein having an amino acid sequence of SEQ ID NO: 123;
A fusion protein having an amino acid sequence of SEQ ID NO: 105 and a fusion protein having an amino acid sequence of SEQ ID NO: 119;
A fusion protein having an amino acid sequence of SEQ ID NO: 105 and a fusion protein having an amino acid sequence of SEQ ID NO: 118;
A fusion protein having an amino acid sequence of SEQ ID NO: 105 and a fusion protein having an amino acid sequence of SEQ ID NO: 121;
A fusion protein having an amino acid sequence of SEQ ID NO: 105 and a fusion protein having an amino acid sequence of SEQ ID NO: 122;
A fusion protein having an amino acid sequence of SEQ ID NO: 106 and a fusion protein having an amino acid sequence of SEQ ID NO: 119;
A fusion protein having an amino acid sequence of SEQ ID NO: 106 and a fusion protein having an amino acid sequence of SEQ ID NO: 118;
A fusion protein having an amino acid sequence of SEQ ID NO: 106 and a fusion protein having an amino acid sequence of SEQ ID NO: 121;
A fusion protein having an amino acid sequence of SEQ ID NO: 106 and a fusion protein having an amino acid sequence of SEQ ID NO: 122;
A fusion protein having an amino acid sequence of SEQ ID NO: 106 and a fusion protein having an amino acid sequence of SEQ ID NO: 124;
A fusion protein having an amino acid sequence of SEQ ID NO: 111 and a fusion protein having an amino acid sequence of SEQ ID NO: 120;
A fusion protein having an amino acid sequence of SEQ ID NO: 111 and a fusion protein having an amino acid sequence of SEQ ID NO: 118;
fusion protein having an amino acid sequence of SEQ ID NO: 107 and a fusion protein having an amino acid sequence of SEQ ID NO: 118;
A fusion protein having an amino acid sequence of SEQ ID NO: 108 and a fusion protein having an amino acid sequence of SEQ ID NO: 120;
A fusion protein having an amino acid sequence of SEQ ID NO: 108 and a fusion protein having an amino acid sequence of SEQ ID NO: 119;
A fusion protein having an amino acid sequence of SEQ ID NO: 108 and a fusion protein having an amino acid sequence of SEQ ID NO: 118;
A fusion protein having an amino acid sequence of SEQ ID NO: 108 and a fusion protein having an amino acid sequence of SEQ ID NO: 121;
A fusion protein having an amino acid sequence of SEQ ID NO: 108 and a fusion protein having an amino acid sequence of SEQ ID NO: 122;
A fusion protein having an amino acid sequence of SEQ ID NO: 108 and a fusion protein having an amino acid sequence of SEQ ID NO: 124; A fusion protein having an amino acid sequence of SEQ ID NO: 112 and a fusion protein having an amino acid sequence of SEQ ID NO: 120;
A fusion protein having an amino acid sequence of SEQ ID NO: 112 and a fusion protein having an amino acid sequence of SEQ ID NO: 118;
A fusion protein having an amino acid sequence of SEQ ID NO: 112 and a fusion protein having an amino acid sequence of SEQ ID NO: 121;
A fusion protein having an amino acid sequence of SEQ ID NO: 112 and a fusion protein having an amino acid sequence of SEQ ID NO: 124;
A fusion protein having an amino acid sequence of SEQ ID NO: 114 and a fusion protein having an amino acid sequence of SEQ ID NO: 115;
A fusion protein having an amino acid sequence of SEQ ID NO: 114 and a fusion protein having an amino acid sequence of SEQ ID NO: 116;
A fusion protein having an amino acid sequence of SEQ ID NO: 109 and a fusion protein having an amino acid sequence of SEQ ID NO: 117;
A fusion protein having an amino acid sequence of SEQ ID NO: 109 and a fusion protein having an amino acid sequence of SEQ ID NO: 115;
A fusion protein having an amino acid sequence of SEQ ID NO: 109 and a fusion protein having an amino acid sequence of SEQ ID NO: 116; and
A fusion protein having an amino acid sequence of SEQ ID NO: 110 and a fusion protein having an amino acid sequence of SEQ ID NO: 115.

In certain other embodiments, the base editor (or base editing composition) may comprise a combination of a fusion protein having an amino acid sequence selected from the group consisting of SEQ ID NOs: 66 to 69 and a fusion protein having an amino acid sequence selected from the group consisting of SEQ ID NOs: 70 to 77.

For example, a base editor (or base editing composition) may comprise a combination of fusion proteins selected from the group consisting of f the following pairs of fusion proteins:

A fusion protein having an amino acid sequence of SEQ ID NO: 66 and a fusion protein having an amino acid sequence of SEQ ID NO: 70;
A fusion protein having an amino acid sequence of SEQ ID NO: 67 and a fusion protein having an amino acid sequence of SEQ ID NO: 70;
A fusion protein having an amino acid sequence of SEQ ID NO: 66 and a fusion protein having an amino acid sequence of SEQ ID NO: 71;
A fusion protein having an amino acid sequence of SEQ ID NO: 67 and a fusion protein having an amino acid sequence of SEQ ID NO: 71;
A fusion protein having an amino acid sequence of SEQ ID NO: 66 and a fusion protein having an amino acid sequence of SEQ ID NO: 72;
A fusion protein having an amino acid sequence of SEQ ID NO: 66 and a fusion protein having an amino acid sequence of SEQ ID NO: 73;
A fusion protein having an amino acid sequence of SEQ ID NO: 67 and a fusion protein having an amino acid sequence of SEQ ID NO: 72;
A fusion protein having an amino acid sequence of SEQ ID NO: 67 and a fusion protein having an amino acid sequence of SEQ ID NO: 73;
A fusion protein having an amino acid sequence of SEQ ID NO: 68 and a fusion protein having an amino acid sequence of SEQ ID NO: 74;
A fusion protein having an amino acid sequence of SEQ ID NO: 68 and a fusion protein having an amino acid sequence of SEQ ID NO: 75;
A fusion protein having an amino acid sequence of SEQ ID NO: 68 and a fusion protein having an amino acid sequence of SEQ ID NO: 76;
A fusion protein having an amino acid sequence of SEQ ID NO: 68 and a fusion protein having an amino acid sequence of SEQ ID NO: 77;
A fusion protein having an amino acid sequence of SEQ ID NO: 69 and a fusion protein having an amino acid sequence of SEQ ID NO: 74;
A fusion protein having an amino acid sequence of SEQ ID NO: 69 and a fusion protein having an amino acid sequence of SEQ ID NO: 75; and
A fusion protein having an amino acid sequence of SEQ ID NO: 69 and a fusion protein having an amino acid sequence of SEQ ID NO: 76.

In certain embodiments, the base editor (or base editing composition) may comprise a combination of polynucleotides comprising a polynucleotide encoding an amino acid sequence selected from the group consisting of SEQ ID NOs: 78 to 85 and a polynucleotide encoding an amino acid sequence selected from the group consisting of SEQ ID NOs: 86 to 99.

For example, the base editor (or base editing composition) may comprise a combination of polynucleotides selected from the group consisting of the following pairs of polynucleotide sequences:
A polynucleotide encoding an amino acid sequence of SEQ ID NO: 78 and a polynucleotide encoding an amino acid sequence of SEQ ID NO: 90;
A polynucleotide encoding an amino acid sequence of SEQ ID NO: 78 and a polynucleotide encoding an amino acid sequence of SEQ ID NO: 91;
A polynucleotide encoding an amino acid sequence of SEQ ID NO: 79 and a polynucleotide encoding an amino acid sequence of SEQ ID NO: 92;
A polynucleotide encoding an amino acid sequence of SEQ ID NO: 79 and a polynucleotide encoding an amino acid sequence of SEQ ID NO: 93;
A polynucleotide encoding an amino acid sequence of SEQ ID NO: 79 and a polynucleotide encoding an amino acid sequence of SEQ ID NO: 94;
A polynucleotide encoding an amino acid sequence of SEQ ID NO: 79 and a polynucleotide encoding an amino acid sequence of SEQ ID NO: 90;
A polynucleotide encoding an amino acid sequence of SEQ ID NO: 79 and a polynucleotide encoding an amino acid sequence of SEQ ID NO: 91;
A polynucleotide encoding an amino acid sequence of SEQ ID NO: 80 and a polynucleotide encoding an amino acid sequence of SEQ ID NO: 95;
A polynucleotide encoding an amino acid sequence of SEQ ID NO: 80 and a polynucleotide encoding an amino acid sequence of SEQ ID NO: 90;
A polynucleotide encoding an amino acid sequence of SEQ ID NO: 80 and a polynucleotide encoding an amino acid sequence of SEQ ID NO: 91;
A polynucleotide encoding an amino acid sequence of SEQ ID NO: 81 and a polynucleotide encoding an amino acid sequence of SEQ ID NO: 96;
A polynucleotide encoding an amino acid sequence of SEQ ID NO: 81 and a polynucleotide encoding an amino acid sequence of SEQ ID NO: 91;
A polynucleotide encoding an amino acid sequence of SEQ ID NO: 82 and a polynucleotide encoding an amino acid sequence of SEQ ID NO: 97;
A polynucleotide encoding an amino acid sequence of SEQ ID NO: 82 and a polynucleotide encoding an amino acid sequence of SEQ ID NO: 90;
A polynucleotide encoding an amino acid sequence of SEQ ID NO: 83 and a polynucleotide encoding an amino acid sequence of SEQ ID NO: 88;
A polynucleotide encoding an amino acid sequence of SEQ ID NO: 83 and a polynucleotide encoding an amino acid sequence of SEQ ID NO: 87;
A polynucleotide encoding an amino acid sequence of SEQ ID NO: 83 and a polynucleotide encoding an amino acid sequence of SEQ ID NO: 89;
A polynucleotide encoding an amino acid sequence of SEQ ID NO: 84 and a polynucleotide encoding an amino acid sequence of SEQ ID NO: 88;
A polynucleotide encoding an amino acid sequence of SEQ ID NO: 84 and a polynucleotide encoding an amino acid sequence of SEQ ID NO: 86;
A polynucleotide encoding an amino acid sequence of SEQ ID NO: 84 and a polynucleotide encoding an amino acid sequence of SEQ ID NO: 87; and
A polynucleotide encoding an amino acid sequence of SEQ ID NO: 85 and a polynucleotide encoding an amino acid sequence of SEQ ID NO: 88.

In certain embodiments, the base editor (or base editing composition) described herein may comprise a combination of a polynucleotide encoding an amino acid sequence selected from the group consisting of SEQ ID NOs: 100 to 114 and a polynucleotide encoding an amino acid sequence selected from the group consisting of SEQ ID NOs: 115 to 125.

For example, the base editor (or base editing composition) described herein may comprise a combination of polynucleotides selected from the group consisting of the following pairs of polynucleotide sequences:

A polynucleotide encoding an amino acid sequence of SEQ ID NO: 100 and a polynucleotide encoding an amino acid sequence of SEQ ID NO: 118;
A polynucleotide encoding an amino acid sequence of SEQ ID NO: 101 and a polynucleotide encoding an amino acid sequence of SEQ ID NO: 119;
A polynucleotide encoding an amino acid sequence of SEQ ID NO: 101 and a polynucleotide encoding an amino acid sequence of SEQ ID NO: 118;
A polynucleotide encoding an amino acid sequence of SEQ ID NO: 102 and a polynucleotide encoding an amino acid sequence of SEQ ID NO: 118;
A polynucleotide encoding an amino acid sequence of SEQ ID NO: 103 and a polynucleotide encoding an amino acid sequence of SEQ ID NO: 120;
A polynucleotide encoding an amino acid sequence of SEQ ID NO: 103 and a polynucleotide encoding an amino acid sequence of SEQ ID NO: 119;
A polynucleotide encoding an amino acid sequence of SEQ ID NO: 103 and a polynucleotide encoding an amino acid sequence of SEQ ID NO: 118;
A polynucleotide encoding an amino acid sequence of SEQ ID NO: 113 and a polynucleotide encoding an amino acid sequence of SEQ ID NO: 118;
A polynucleotide encoding an amino acid sequence of SEQ ID NO: 104 and a polynucleotide encoding an amino acid sequence of SEQ ID NO: 119;
A polynucleotide encoding an amino acid sequence of SEQ ID NO: 104 and a polynucleotide encoding an amino acid sequence of SEQ ID NO: 118;
A polynucleotide encoding an amino acid sequence of SEQ ID NO: 104 and a polynucleotide encoding an amino acid sequence of SEQ ID NO: 121;
A polynucleotide encoding an amino acid sequence of SEQ ID NO: 104 and a polynucleotide encoding an amino acid sequence of SEQ ID NO: 122;
A polynucleotide encoding an amino acid sequence of SEQ ID NO: 105 and a polynucleotide encoding an amino acid sequence of SEQ ID NO: 120;
A polynucleotide encoding an amino acid sequence of SEQ ID NO: 105 and a polynucleotide encoding an amino acid sequence of SEQ ID NO: 123;
A polynucleotide encoding an amino acid sequence of SEQ ID NO: 105 and a polynucleotide encoding an amino acid sequence of SEQ ID NO: 119;
A polynucleotide encoding an amino acid sequence of SEQ ID NO: 105 and a polynucleotide encoding an amino acid sequence of SEQ ID NO: 118;
A polynucleotide encoding an amino acid sequence of SEQ ID NO: 105 and a polynucleotide encoding an amino acid sequence of SEQ ID NO: 121;
A polynucleotide encoding an amino acid sequence of SEQ ID NO: 105 and a polynucleotide encoding an amino acid sequence of SEQ ID NO: 122;
A polynucleotide encoding an amino acid sequence of SEQ ID NO: 106 and a polynucleotide encoding an amino acid sequence of SEQ ID NO: 119;
A polynucleotide encoding an amino acid sequence of SEQ ID NO: 106 and a polynucleotide encoding an amino acid sequence of SEQ ID NO: 118;
A polynucleotide encoding an amino acid sequence of SEQ ID NO: 106 and a polynucleotide encoding an amino acid sequence of SEQ ID NO: 121;
A polynucleotide encoding an amino acid sequence of SEQ ID NO: 106 and a polynucleotide encoding an amino acid sequence of SEQ ID NO: 122;
A polynucleotide encoding an amino acid sequence of SEQ ID NO: 106 and a polynucleotide encoding an amino acid sequence of SEQ ID NO: 124;
A polynucleotide encoding an amino acid sequence of SEQ ID NO: 111 and a polynucleotide encoding an amino acid sequence of SEQ ID NO: 120;
A polynucleotide encoding an amino acid sequence of SEQ ID NO: 111 and a polynucleotide encoding an amino acid sequence of SEQ ID NO: 118;
A polynucleotide encoding an amino acid sequence of SEQ ID NO: 107 and a polynucleotide encoding an amino acid sequence of SEQ ID NO: 118;
A polynucleotide encoding an amino acid sequence of SEQ ID NO: 108 and a polynucleotide encoding an amino acid sequence of SEQ ID NO: 120;
A polynucleotide encoding an amino acid sequence of SEQ ID NO: 108 and a polynucleotide encoding an amino acid sequence of SEQ ID NO: 119;
A polynucleotide encoding an amino acid sequence of SEQ ID NO: 108 and a polynucleotide encoding an amino acid sequence of SEQ ID NO: 118;
A polynucleotide encoding an amino acid sequence of SEQ ID NO: 108 and a polynucleotide encoding an amino acid sequence of SEQ ID NO: 121;
A polynucleotide encoding an amino acid sequence of SEQ ID NO: 108 and a polynucleotide encoding an amino acid sequence of SEQ ID NO: 122;
A polynucleotide encoding an amino acid sequence of SEQ ID NO: 108 and a polynucleotide encoding an amino acid sequence of SEQ ID NO: 124;
A polynucleotide encoding an amino acid sequence of SEQ ID NO: 112 and a polynucleotide encoding an amino acid sequence of SEQ ID NO: 120;
A polynucleotide encoding an amino acid sequence of SEQ ID NO: 112 and a polynucleotide encoding an amino acid sequence of SEQ ID NO: 118;
A polynucleotide encoding an amino acid sequence of SEQ ID NO: 112 and a polynucleotide encoding an amino acid sequence of SEQ ID NO: 121;
A polynucleotide encoding an amino acid sequence of SEQ ID NO: 112 and a polynucleotide encoding an amino acid sequence of SEQ ID NO: 124;
A polynucleotide encoding an amino acid sequence of SEQ ID NO: 114 and a polynucleotide encoding an amino acid sequence of SEQ ID NO: 115;
A polynucleotide encoding an amino acid sequence of SEQ ID NO: 114 and a polynucleotide encoding an amino acid sequence of SEQ ID NO: 116;
A polynucleotide encoding an amino acid sequence of SEQ ID NO: 109 and a polynucleotide encoding an amino acid sequence of SEQ ID NO: 117;
A polynucleotide encoding an amino acid sequence of SEQ ID NO: 109 and a polynucleotide encoding an amino acid sequence of SEQ ID NO: 115;
A polynucleotide encoding an amino acid sequence of SEQ ID NO: 109 and a polynucleotide encoding an amino acid sequence of SEQ ID NO: 116; and
A polynucleotide encoding an amino acid sequence of SEQ ID NO: 110 and a polynucleotide encoding an amino acid sequence of SEQ ID NO: 115.

The base editor (or base editing composition) described herein may comprise a combination of a polynucleotide encoding an amino acid sequence selected from the group consisting of SEQ ID NOs: 66 to 69 and a polynucleotide encoding an amino acid sequence selected from the group consisting of SEQ ID NOs: 70 to 77.

For example, the base editor (or base editing composition) described herein may comprise a combination of polynucleotides selected from the group consisting of the following pairs of polynucleotide sequences:

A polynucleotide encoding an amino acid sequence of SEQ ID NO: 66 and a polynucleotide encoding an amino acid sequence of SEQ ID NO: 70;
A polynucleotide encoding an amino acid sequence of SEQ ID NO: 67 and a polynucleotide encoding an amino acid sequence of SEQ ID NO: 70;
A polynucleotide encoding an amino acid sequence of SEQ ID NO: 66 and a polynucleotide encoding an amino acid sequence of SEQ ID NO: 71;
A polynucleotide encoding an amino acid sequence of SEQ ID NO: 67 and a polynucleotide encoding an amino acid sequence of SEQ ID NO: 71;
A polynucleotide encoding an amino acid sequence of SEQ ID NO: 66 and a polynucleotide encoding an amino acid sequence of SEQ ID NO: 72;
A polynucleotide encoding an amino acid sequence of SEQ ID NO: 66 and a polynucleotide encoding an amino acid sequence of SEQ ID NO: 73;
A polynucleotide encoding an amino acid sequence of SEQ ID NO: 67 and a polynucleotide encoding an amino acid sequence of SEQ ID NO: 72;
A polynucleotide encoding an amino acid sequence of SEQ ID NO: 67 and a polynucleotide encoding an amino acid sequence of SEQ ID NO: 73;
A polynucleotide encoding an amino acid sequence of SEQ ID NO: 68 and a polynucleotide encoding an amino acid sequence of SEQ ID NO: 74;
A polynucleotide encoding an amino acid sequence of SEQ ID NO: 68 and a polynucleotide encoding an amino acid sequence of SEQ ID NO: 75;
A polynucleotide encoding an amino acid sequence of SEQ ID NO: 68 and a polynucleotide encoding an amino acid sequence of SEQ ID NO: 76;
A polynucleotide encoding an amino acid sequence of SEQ ID NO: 68 and a polynucleotide encoding an amino acid sequence of SEQ ID NO: 77;
A polynucleotide encoding an amino acid sequence of SEQ ID NO: 69 and a polynucleotide encoding an amino acid sequence of SEQ ID NO: 74;
A polynucleotide encoding an amino acid sequence of SEQ ID NO: 69 and a polynucleotide encoding an amino acid sequence of SEQ ID NO: 75; and
A polynucleotide encoding an amino acid sequence of SEQ ID NO: 69 and a polynucleotide encoding an amino acid sequence of SEQ ID NO: 76.

Certain further embodiments relate to a method of correcting a mitochondrial DNA base mutation in a patient with LHON, comprising contacting the mitochondrial DNA of the patient with a mitochondrial DNA base editor (or a base editing composition; for example, a combination of fusion proteins or polynucleotides described under the section “Composition Examples of Base Editor (or Base Editing Composition) According to the Present Invention”), wherein the correction involves correcting adenine (A) at position 3460 of mitochondrial ND1 DNA to guanine (G), or correcting adenine (A) at position 11778 of mitochondrial ND4 DNA to guanine (G), or correcting cytosine (A) at position 14484 of mitochondrial ND6 DNA to thymine (T).

The mitochondrial DNA base editor may correct adenine (A) at position 3460 of mitochondrial ND1 DNA to guanine (G), or adenine (A) at position 11778 of mitochondrial ND4 DNA to guanine (G), or cytosine (A) at position 14484 of mitochondrial ND6 DNA to thymine (T), with a frequency of 0.5% or more, 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, or 20% or more. In certain embodiments, the correction of adenine (A) at position 3460 of mitochondrial ND1 DNA, adenine (A) at position 11778 of mitochondrial ND4 DNA, or cytosine (A) at position 14484 of mitochondrial ND6 DNA means that the corresponding base has changed as compared to the base sequence of the mitochondrial DNA that has not been contacted with the base editing composition described herein. Whether the base has changed may be confirmed by DNA sequencing.

Described herein is a method for preventing or treating Leber hereditary optic neuropathy (LHON), comprising administering to a patient in need of prevention or treatment of LHON an effective amount of a mitochondrial DNA base editor (or base editing composition; for example, a combination of fusion proteins or a combination of polynucleotides described under the section “Composition examples of Base Editor (or Dase Editing Composition) described herein”), that is, a fusion protein (including a combination of one or more different fusion proteins) capable of correcting adenine (A) at position 3460 of mitochondrial ND1 DNA of a patient with LHON to guanine (G), or correcting adenine (A) at position 11778 of mitochondrial ND4 DNA to guanine (G), or correcting cytosine (A) at position 14484 of mitochondrial ND6 DNA to thymine (T), or a polynucleotide comprising a gene encoding the fusion protein (including a combination of different polynucleotides each encoding one or more different fusion proteins).

The term “effective amount” as used herein refers to an amount of a biologically active agent sufficient to induce a desired biological response. In some embodiments, the effective amount is the amount necessary to improve symptoms of the disease in an untreated patient. A therapeutic method of treating a disease and the effective amount of the active ingredient used in the treatment may vary depending on the method of administration and a subject's age, weight, and general health. In one embodiment, an effective amount is an amount of a base editor (e.g., a fusion protein, or polynucleotide, or a vector or lipid nanoparticle comprising the same) described herein sufficient to introduce a change in a gene of interest (e.g., mitochondrial DNA) in a cell (e.g., in vitro, in vivo or ex vivo). In one embodiment, the effective amount is the amount of the base editor (a fusion protein, or a polynucleotide, or a vector or lipid nanoparticle comprising the same) necessary to achieve a therapeutic effect (for example, to reduce or control symptoms or conditions of a patient with LHON). Such an therapeutic effect need not be sufficient to alter all mitochondrial DNA in all cells of the subject, tissue or organ, but may be sufficient to alter mitochondrial DNA in at least about 1%, 5%, 10%, 25%, 50%, 75%, or more of the cells present in the subject, tissue or organ, or may be sufficient to alter mitochondrial DNA in at least about 1%, 5%, 10%, 25%, 50%, 75%, or more of the total number of copies of mitochondrial DNA present in the corresponding cells. In one embodiment, the effective amount is sufficient to improve one or more symptoms of LHON.

Described herein is a pharmaceutical composition for preventing or treating LHON comprising an effective amount of the base editing composition or the polynucleotide. Described herein is a pharmaceutical composition for preventing or treating LHON, comprising a mitochondrial DNA base editor described herein, i.e., a fusion protein (including a combination of one or more different fusion proteins) capable of editing adenine (A) at position 3460 of mitochondrial ND1 DNA of a patient with LHON to guanine (G), adenine (A) at position 11778 of mitochondrial ND4 DNA to guanine (G), or cytosine (A) at position 14484 of mitochondrial ND6 DNA to thymine (T), or a polynucleotide comprising a gene encoding the fusion protein (including a combination of different polynucleotides each encoding one or more different fusion proteins), and a pharmaceutically acceptable excipient, carrier or vehicle.

As used herein, the term “pharmaceutical composition” means a composition formulated for pharmaceutical use. In some embodiments, the pharmaceutical composition further comprises a pharmaceutically acceptable carrier. Those skilled in the pharmaceutical art are well aware of pharmaceutical carriers which may be generally used to formulate a base editing composition described herein for pharmaceutical uses. In some embodiments, the pharmaceutical composition may comprise an additional agent (e.g., an agent for specific delivery, increasing half-life, or other therapeutic compounds). The term “pharmaceutically acceptable carrier” means a pharmaceutically acceptable substance, composition, or vehicle, such as a liquid or solid filler, diluent, excipient, manufacturing aid, or solvent encapsulating material, which carries or transports a compound from one site in the body (e.g., a site of delivery) to another site (e.g., an organ, tissue, or part of the body). A pharmaceutically acceptable carrier is “acceptable” in the sense that it is compatible with the other ingredients of the formulation and not deleterious to the tissues of the subject (e.g., in terms of physiological compatibility, sterility, physiological pH, etc.). The terms “excipient”, “carrier”, “pharmaceutically acceptable carrier”, “vehicle”, and the like may be used interchangeably.

Described herein is a gene delivery vehicle comprising a polynucleotide encoding any one or more fusion proteins included in a base editing composition described herein. The gene delivery vehicle may be a viral vector, preferably an adeno-associated viral vector. The gene delivery vehicle may also be in the form of a lipid nanoparticle or a polymeric nanoparticle. For example, a polynucleotide or combination of polynucleotides described herein may form a complex with a lipid or a polymer.

Described herein is a gene therapy agent for preventing or treating LHON, comprising the gene delivery vehicle. The pharmaceutical composition described above may be in the form of a gene therapy product comprising, as an active ingredient, a polynucleotide (including a combination of different polynucleotides each encoding one or more different fusion proteins) encoding a fusion protein (including a combination of one or more different fusion proteins) used as a mitochondrial DNA base editor described herein.

In certain embodiments, a polynucleotide comprising a gene encoding a fusion protein (including a combination of one or more different fusion proteins) used as a mitochondrial DNA base editor may be delivered to a patient, and as such delivery methods, a method using a virus as a vector, a non-viral method using a synthetic phospholipid, synthetic cationic polymer, or the like, an electroporation method in which a gene is introduced by temporarily stimulating the cell membrane electrically, and the like may be used. Among the above methods, when a virus is used as a vector, a virus with a low gene loading capacity (for example, an adeno-associated virus (AAV) with a size of approximately 4.7 kbp) has a limitation in use due to the size of a DNA editing fusion protein, but if a zinc finger protein is used as the DNA binding protein or a full-length deaminase is used as the deaminase, such a virus may be used as a vector. The vector may be an adeno-associated virus.

The present invention may relate to (1) to (35) below based on the contents described above, but is not limited thereto.

(1) A base editing composition capable of correcting a mitochondrial DNA mutation in a patient with Leber hereditary optic neuropathy (LHON), comprising one or more fusion proteins, wherein each of the one or more fusion proteins independently comprises DNA binding protein that specifically binds to mitochondrial DNA of a patient with LHON and further comprises at least one of adenine deaminase and cytosine deaminase, and wherein cytosine deaminase is present in a full-length form or in the form of two splits.

(2) In the base editing composition according to (1), wherein the composition is capable of editing adenine (A) at position 3460 of mitochondrial ND1 DNA to guanine (G), adenine (A) at position 11778 of mitochondrial ND4 DNA to guanine (G), or cytosine (A) at position 14484 of mitochondrial ND6 DNA to thymine (T) in a patient with LHON.

(3) In the base editing composition according to (1) or (2), wherein the cytosine deaminase is APOBEC (apolipoprotein B editing complex), AID (activation-induced deaminase), TadA (tRNA-specific adenosine deaminase) or DddAₜₒₓ, or a variant thereof.

(4) The base editing composition according to (1) or (2), wherein cytosine deaminase is DddAₜₒₓ and is included in the form of a first split and a second split, and wherein one or more amino acids located on the interface between the first and second splits are substituted with other amino acids.

(5) The base editing composition according to (4), wherein the first split of DddAₜₒₓ comprises an amino acid sequence of SEQ ID NO: 5 or 139 or a variant thereof, wherein the variant has at least one amino acid selected from the group consisting of positions 87, 88, 91, 92, 95, 100, 101, 102, and 103 of the amino acid sequence of SEQ ID NO: 5 substituted with another amino acid, and the second split of DddAₜₒₓ comprises an amino acid sequence of SEQ ID NO: 6 or 140 or a variant thereof, wherein the variant has at least one amino acid selected from the group consisting of positions 13, 14, 15, and 16 of the amino acid sequence of SEQ ID NO: 6 substituted with another amino acid.

(6) The base editing composition according to (1) or (2), wherein the adenine deaminase is APOBEC, AID or TadA, or a variant thereof.

(7) The base editing composition according to (6), wherein the adenine deaminase comprises the amino acid sequence of SEQ ID NO: 1 or a conservative amino acid substitution thereof.

(8) The base editing composition according to any one of (1) to (7), wherein the DNA binding protein is selected from the group consisting of a zinc finger protein, a TALE protein, and a CRISPR-associated nuclease.

(9) The base editing composition according to any one of (1) to (8), wherein each of the one or more fusion proteins independently comprises UGI (uracil glycosylase inhibitor).

(10) The base editing composition according to any one of (1) to (9), wherein each of the one or more fusion proteins independently comprises a nuclear export signal (NES).

(11) The base editing composition according to any one of claims 1) to (10), wherein each of the one or more fusion proteins independently comprises a mitochondrial targeting sequence (MTS).

(12) The base editing composition according to any one of (1) to (8), wherein one DNA binding protein binds to a nucleotide sequence of mitochondrial ND1 DNA: 5′-CAAACTCAAACTACGAACGCACTCACAGTCACATCATAATCCTCTCTCAAGGACTT CAAAC-3′ or a portion thereof.

(13) The base editing composition according to any one of (1) to (8), wherein one DNA binding protein binds to a nucleotide sequence of mitochondrial ND1 DNA:

5′-CAAACTCAAACTACGAACGCACTCACAGTCACATCATAATCCTCTCTCAAGGACTT CAAAC-3′ or a portion thereof.

(14) The base editing composition according to any one of (1) to (8), wherein one DNA binding protein binds to a nucleotide sequence of mitochondrial ND1 DNA: 5′-TCGCTGTAGTATATCCAAAGACAACCACCATTCCCCCTAAATAAATTAAAAAAACT-3′ or a portion thereof.

(15) The base editing composition according to any one of (1) to (8), wherein one DNA binding protein comprises an amino acid sequence selected from the group consisting of SEQ ID NOs: 7 to 65, or a conservative amino acid substitution thereof.

(16) The base editing composition according to (1), wherein the composition comprises two fusion proteins and is capable of editing adenine (A) at position 3460 of mitochondrial ND1 DNA to guanine (G) in a patient with LHON, wherein each of the two fusion proteins comprises DddAₜₒₓ split and TALE protein that specifically binds to mitochondrial ND1 DNA, and one of the two fusion proteins further comprises TadA8e.

(17) The base editing composition according to (1), wherein the composition comprises two fusion proteins and is capable of editing adenine (A) at position 11778 of mitochondrial ND4 DNA to guanine (G) in a patient with LHON, wherein each of the two fusion proteins comprises DddAₜₒₓ split and TALE protein or zinc finger protein that specifically binds to mitochondrial ND4 DNA, and one of the two fusion proteins further comprises TadA8e.

(18) The base editing composition according to (1), wherein two fusion proteins, wherein the composition comprises two fusion proteins and is capable of editing cytosine (A) at position 14484 of mitochondrial ND6 DNA to thymine (T) in a patient with LHON, wherein each of the two fusion proteins comprises DddAₜₒₓ split and TALE protein that specifically binds to mitochondrial ND6.

(19) The base editing composition according to (16), wherein one fusion protein comprises TALE protein comprising an amino acid sequence selected from the group consisting of SEQ ID NOs: 10 to 17 or conservative amino acid substitution thereof, and the other fusion protein comprises TALE protein comprising an amino acid sequence selected from the group consisting of SEQ ID NOs: 18 to 31 or a conservative amino acid substitution thereof.

(20) The base editing composition according to (17), wherein one fusion protein comprises TALE protein comprising an amino acid sequence selected from the group consisting of SEQ ID NOs: 32 to 42 or zinc finger protein comprising an amino acid sequence selected from the group consisting of SEQ ID NOs: 7 to 9 or conservative amino acid substitution thereof, and the other fusion protein comprises TALE protein comprising an amino acid sequence selected from the group consisting of SEQ ID NOs: 43 to 53 or zinc finger protein comprising an amino acid sequence selected from the group consisting of SEQ ID NOs: 7 to 9 or a conservative amino acid substitution thereof.

(21) The base editing composition according to (18), wherein one fusion protein comprises a TALE protein comprising an amino acid sequence selected from the group consisting of SEQ ID NOs: 54 to 57 or a conservative amino acid substitution thereof, and the other fusion protein comprises a TALE protein comprising an amino acid sequence selected from the group consisting of SEQ ID NOs: 58 to 65 or a conservative amino acid substitution thereof.

(22) The base editing composition according to (16) or (19), comprising a combination of a fusion protein comprising an amino acid sequence selected from the group consisting of SEQ ID NOs: 78 to 85 and a fusion protein comprising an amino acid sequence selected from the group consisting of SEQ ID NOs: 86 to 99.

(23) The base editing composition according to (17) or (20), comprising a combination of a fusion protein comprising an amino acid sequence selected from the group consisting of SEQ ID NOs: 100 to 114 and a fusion protein comprising an amino acid sequence selected from the group consisting of SEQ ID NOs: 115 to 125.

(24) The base editing composition according to (18) or (21), comprising a combination of a fusion protein comprising an amino acid sequence selected from the group consisting of SEQ ID NOs: 66 to 69 and a fusion protein comprising an amino acid sequence selected from the group consisting of SEQ ID NOs: 70 to 77.

(25) A polynucleotide encoding any one of the fusion proteins included in the base editing composition according to any one of (1) to (24), or a combination of two or more of said polynucleotides.

(26) The combination of polynucleotides according to (25), comprising a polynucleotide encoding an amino acid sequence selected from the group consisting of SEQ ID NOs: 78 to 85 and a polynucleotide encoding an amino acid sequence selected from the group consisting of SEQ ID NOs: 86 to 99.

(27) The combination of polynucleotides according to (25), comprising a polynucleotide encoding an amino acid sequence selected from the group consisting of SEQ ID NOs: 100 to 114 and a polynucleotide encoding an amino acid sequence selected from the group consisting of SEQ ID NOs: 115 to 125.

(28) The combination of polynucleotides according to (25), comprising a polynucleotide encoding an amino acid sequence selected from the group consisting of SEQ ID NOs: 66 to 69 and a polynucleotide encoding an amino acid sequence selected from the group consisting of SEQ ID NOs: 70 to 77.

(29) A method of correcting a mitochondrial DNA mutation in a patient with LHON, the method comprising contacting mitochondrial DNA of the patient with LHON with a base editing composition according to any one of (1) to (24), wherein the correction involves editing of guanine (G) at position 3460 of mitochondrial ND1 DNA to adenine (A), guanine (G) at position 11778 of mitochondrial ND4 DNA to adenine (A), or cytosine (A) at position 14484 of mitochondrial ND6 DNA to thymine (T).

(30) A method of preventing or treating LHON, comprising administering to a patient in need of prevention or treatment of LHON a base editing composition according to any one of (1) to (24) or a composition comprising a polynucleotide or a combination of polynucleotides according to any one of (25) to (28).

(31) A pharmaceutical composition for preventing or treating LHON, comprising a base editing composition according to any one of claims 1) to (24) or a polynucleotide or combination of polynucleotides according to any one of claims 25) to (28).

(32) gene delivery vehicle comprising a polynucleotide or a combination of polynucleotides according to any one of (25) to (28).

(33) The gene delivery vehicle according to (32), wherein the gene delivery vehicle is an adeno-associated virus vector.

(34) The gene delivery vehicle according to (32), wherein the gene delivery vehicle is a lipid nanoparticle or a polymeric nanoparticle.

(35) A gene therapy agent for preventing or treating LHON, comprising a gene delivery vehicle according to (32) to (34).

Hereinafter, embodiments of the present invention will be described. However, the following examples are provided only to illustrate the present invention, and should not be construed as limiting the scope of the present invention.

### Example 1: Amplification of Template DNA Having the m.T14484C Mutation

A double-stranded DNA sequence mimicking the mitochondrial genome of a patient with the m. T14484C mutation was synthesized as a gBlock DNA fragment from IDT (Integrated DNA Technologies). The sequence of the obtained template DNA is as follows.

Template DNA was amplified using the forward primer (GACTGGTTCCAATTGACAACG) and reverse primer (GCAAATGGCATTCTGACATCC), and then purified using a PCR purification kit (Geneall). It was freshly diluted in distilled water to a concentration of 10 ng/μL just before use in the experiment.

### Example 2: Synthesis of Fusion Proteins to Correct the m.T14484C Mutation

After adjusting the concentration of the template DNA obtained in Example 1 to 10 ng/μL, a reaction mixture containing 10 ng of template DNA, 0.5 ug of a plasmid containing DNA encoding the first fusion protein, 0.5 ug of a plasmid containing DNA encoding the second fusion protein, and 20 μL of an in vitro coupled transcription/translation (IVTT) kit mixture (including distilled water up to 25 uL) was mixed in a tube, and then reacted at 30° C. for 6 hours and then at 37° C. for 16 hours.

The first fusion protein and the second fusion protein were linked in the order of [MTS]-[tag]-[TALE protein]-[linker]-[DddAₜₒₓ split]-[linker]-[UGI]. The tag is 3X HA (SEQ ID NO: 136) or 3X FLAG (SEQ ID NO: 137). The proteins used are located between the CMV promoter and the T7 promoter and terminator sequence.

### Example 3: Sequencing and Base Editing Efficiency Measurement to Confirm Correction of m.T14484C Mutation

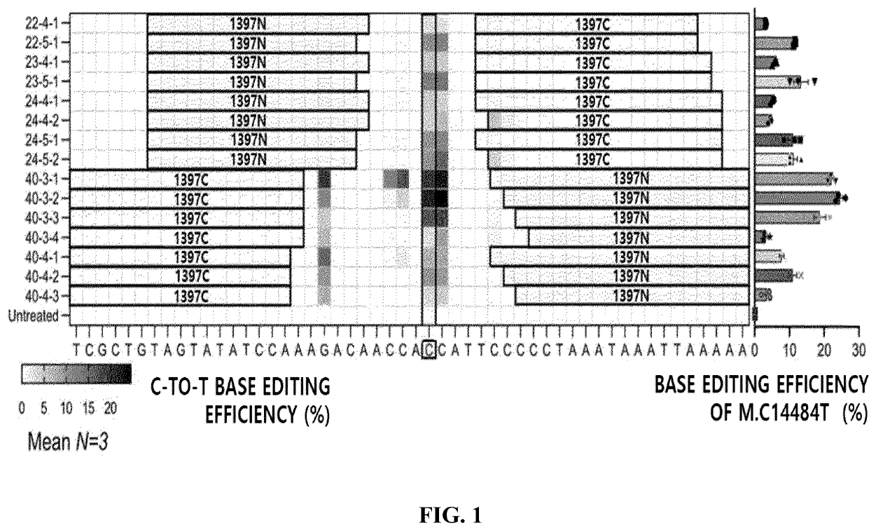

The reaction product obtained in Example 2 was as a template without purification, and the sequence was analyzed using a targeted deep sequencing technique. The efficiency of the base editors developed for m.C14484T correction was screened, and the DNA binding sites and base editing efficiencies of 15 base editors with high efficiency are shown in FIG. 1.

The amino acid sequences of the first fusion protein (including the left TALE) and the second fusion protein (including the right TALE) used in the 15 base editors that were confirmed to have high base editing efficiency are as follows.

First Fusion Protein
SEQ ID NO: 66
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPAQVVAIASNIGGKQALETVQRL
LPVLCQAHGLTPEQVVAIASNNGGKQALETVQRLLPVLCQAHGLT
PDQVVAIASNGGGKQALETVQRLLPVLCQDHGLTPAQVVAIASNI
GGKQALETVQRLLPVLCQDHGLTPAQVVAIASNGGGKQALETVQR
LLPVLCQDHGLTPDQVVAIASNIGGKQALETVQRLLPVLCQDHGL
TPAQVVAIASNGGGKQALETVQRLLPVLCQDHGLTPAQVVAIASH
DGGKQALETVQRLLPVLCQAHGLTPEQVVAIASHDGGKQALETVQ
RLLPVLCQAHGLTPDQVVAIASNIGGKQALETVQRLLPVLCQAHG
LTPAQVVAIASNIGGKQALETVQRLLPVLCQDHGLTPAQVVAIAS
NIGGKQALETVQRLLPVLCQAHGLTPAQVVAIASNNGGKQALETV
QRLLPVLCQAHGLTPAQVVAIASNIGGKQALETVQRLLPVLCQAH
GLTPAQVVAIASHDGGKQALETVQRLLPVLCQAHGLTPEQVVAIA
SNGGGKQALESIVAQLSRPDPALAALTNDHLVALACLGGRPALDA
VKKGLGGSGSGSYALGPYQISAPQLPAYNGQTVGTFYYVNDAGGL
ESKVFISGGPTPYPNYVSAGHVEGQSALFMRDNGISEGLVFHNNP
KGTCGFCVNMIETLLPENAAMTVVPPEGSGGSTNLSDIIEKETGK
QLVIQESILMLPEEVEEVIGNKPESDILVHTAYDESTDENVMLLT
SDAPEYKPWALVIQDSNGENKIKML
(The underlined portion corresponds to the TALE
protein including the N-terminal domain and
C-terminal domain (including the half domain))
SEQ ID NO: 67
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPAQVVAIASNIGGKQALETVQRL
LPVLCQAHGLTPEQVVAIASNNGGKQALETVQRLLPVLCQAHGLT
PDQVVAIASNGGGKQALETVQRLLPVLCQDHGLTPAQVVAIASNI
GGKQALETVQRLLPVLCQDHGLTPAQVVAIASNGGGKQALETVQR
LLPVLCQDHGLTPDQVVAIASNIGGKQALETVQRLLPVLCQDHGL
TPAQVVAIASNGGGKQALETVQRLLPVLCQDHGLTPAQVVAIASH
DGGKQALETVQRLLPVLCQAHGLTPEQVVAIASHDGGKQALETVQ
RLLPVLCQAHGLTPDQVVAIASNIGGKQALETVQRLLPVLCQAHG
LTPAQVVAIASNIGGKQALETVQRLLPVLCQDHGLTPAQVVAIAS
NIGGKQALETVQRLLPVLCQAHGLTPDQVVAIASNNGGKQALETV
QRLLPVLCQAHGLTPDQVVAIASNIGGKQALETVQRLLPVLCQAH
GLTPEQVVAIASNGGGKQALESIVAQLSRPDPALAALTINDHLVA
LACLGGRPALDAVKKGLGGSGSGSYALGPYQISAPQLPAYNGQTV
GTFYYVNDAGGLESKVFISGGPTPYPNYVSAGHVEGQSALFMRDN
GISEGLVFHNNPKGTCGFCVNMIETLLPENAAMTVVPPEGSGGST
NLSDIIEKETGKQLVIQESILMLPEEVEEVIGNKPESDILVHTAY
DESTDENVMLLTSDAPEYKPWALVIQDSNGENKIKML.
(The underlined portion corresponds to the TALE
protein including the N-terminal domain and
C-terminal domain (including the half domain))
SEQ ID NO: 68
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPAQVVAIASHDGGKQALETVQRLLPVLCQAH
GLTPDQVVAIASNNGGKQALETVQRLLPVLCQDHGLTPDQVVAIA
SHDGGKQALETVQRLLPVLCQDHGLTPAQVVAIASNGGGKQALET
VQRLLPVLCQAHGLTPDQVVAIASNNGGKQALETVQRLLPVLCQA
HGLTPAQVVAIASNGGGKQALETVQRLLPVLCQDHGLTPAQVVAI
ASNIGGKQALETVQRLLPVLCQAHGLTPEQVVAIASNNGGKQALE
TVQRLLPVLCQAHGLTPDQVVAIASNGGGKQALETVQRLLPVLCQ
AHGLTPAQVVAIASNIGGKQALETVQRLLPVLCQDHGLTPAQVVA
IASNGGGKQALETVQRLLPVLCQDHGLTPDQVVAIASNIGGKQAL
ETVQRLLPVLCQDHGLTPAQVVAIASNGGGKQALETVQRLLPVLC
QDHGLTPEQVVAIASHDGGKQALETVQRLLPVLCQAHGLTPAQVV
AIASHDGGKQALETVQRLLPVLCQDHGLTPDQVVAIASNIGGKQA
LETVQRLLPVLCQAHGLTPEQVVAIASNGGGKQALESIVAQLSRP
DPALAALTNDHLVALACLGGRPALDAVKKGLGGSGSAIPVKRGAT
GETKVFIGNSNSPKSPTKGGCSGGSTNLSDIIEKETGKQLVIQES
ILMLPEEVEEVIGNKPESDILVHTAYDESTDENVMLLTSDAPEYK
PWALVIQDSNGENKIKML.
(The underlined portion corresponds to the
TALE protein including the N-terminal domain
and C-terminal domain (including the half
domain))
SEQ ID NO: 69
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPAQVVAIASHDGGKQALETVQRLLPVLCQAH
GLTPDQVVAIASNNGGKQALETVQRLLPVLCQDHGLTPDQVVAIA
SHDGGKQALETVQRLLPVLCQDHGLTPAQVVAIASNGGGKQALET
VQRLLPVLCQAHGLTPDQVVAIASNNGGKQALETVQRLLPVLCQA
HGLTPAQVVAIASNGGGKQALETVQRLLPVLCQDHGLTPAQVVAI
ASNIGGKQALETVQRLLPVLCQAHGLTPEQVVAIASNNGGKQALE
TVQRLLPVLCQAHGLTPDQVVAIASNGGGKQALETVQRLLPVLCQ
AHGLTPAQVVAIASNIGGKQALETVQRLLPVLCQDHGLTPAQVVA
IASNGGGKQALETVQRLLPVLCQDHGLTPDQVVAIASNIGGKQAL
ETVQRLLPVLCQDHGLTPAQVVAIASNGGGKQALETVQRLLPVLC
QDHGLTPEQVVAIASHDGGKQALETVQRLLPVLCQAHGLTPAQVV
AIASHDGGKQALETVQRLLPVLCQAHGLTPEQVVAIASNGGGKQA
LESIVAQLSRPDPALAALTNDHLVALACLGGRPALDAVKKGLGGS
GSAIPVKRGATGETKVFIGNSNSPKSPTKGGCSGGSTNLSDIIEK
ETGKQLVIQESILMLPEEVEEVIGNKPESDILVHTAYDESTDENV
MLLTSDAPEYKPWALVIQDSNGENKIKML.
(The underlined portion corresponds to
the TALE protein including the N-terminal
domain and C-terminal domain (including the
half domain))
Second Fusion Protein
SEQ ID NO: 70
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPDQVVAIASNIGGKQALETVQRLLPVLCQAH
GLTPDQVVAIASNIGGKQALETVQRLLPVLCQDHGLTPDQVVAIA
SNGGGKQALETVQRLLPVLCQAHGLTPEQVVAIASNGGGKQALET
VQRLLPVLCQAHGLTPDQVVAIASNGGGKQALETVQRLLPVLCQD
HGLTPDQVVAIASNIGGKQALETVQRLLPVLCQDHGLTPDQVVAI
ASNGGGKQALETVQRLLPVLCQAHGLTPAQVVAIASNGGGKQALE
TVQRLLPVLCQAHGLTPEQVVAIASNGGGKQALETVQRLLPVLCQ
AHGLTPAQVVAIASNIGGKQALETVQRLLPVLCQAHGLTPAQVVA
IASNNGGKQALETVQRLLPVLCQDHGLTPAQVVAIASNNGGKQAL
ETVQRLLPVLCQAHGLTPAQVVAIASNNGGKQALETVQRLLPVLC
QAHGLTPAQVVAIASNNGGKQALETVQRLLPVLCQAHGLTPDQVV
AIASNNGGKQALETVQRLLPVLCQAHGLTPEQVVAIASNGGGKQA
LESIVAQLSRPDPALAALTNDHLVALACLGGRPALDAVKKGLGGS
GSAIPVKRGATGETKVFIGNSNSPKSPTKGGCSGGSTNLSDIIEK
ETGKQLVIQESILMLPEEVEEVIGNKPESDILVHTAYDESTDENV
MLLTSDAPEYKPWALVIQDSNGENKIKML.
(The underlined portion corresponds to
the TALE protein including the N-terminal
domain and C-terminal domain (including the
half domain))
SEQ ID NO: 71
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPEQVVAIASNGGGKQALETVQRLLPVLCQAH
GLTPAQVVAIASNIGGKQALETVQRLLPVLCQDHGLTPAQVVAIA
SNIGGKQALETVQRLLPVLCQAHGLTPDQVVAIASNGGGKQALET
VQRLLPVLCQAHGLTPAQVVAIASNGGGKQALETVQRLLPVLCQA
HGLTPEQVVAIASNGGGKQALETVQRLLPVLCQAHGLTPAQVVAI
ASNIGGKQALETVQRLLPVLCQAHGLTPDQVVAIASNGGGKQALE
TVQRLLPVLCQAHGLTPDQVVAIASNGGGKQALETVQRLLPVLCQ
AHGLTPAQVVAIASNGGGKQALETVQRLLPVLCQDHGLTPAQVVA
IASNIGGKQALETVQRLLPVLCQAHGLTPDQVVAIASNNGGKQAL
ETVQRLLPVLCQAHGLTPAQVVAIASNNGGKQALETVQRLLPVLC
QAHGLTPAQVVAIASNNGGKQALETVQRLLPVLCQAHGLTPAQVV
AIASNNGGKQALETVQRLLPVLCQAHGLTPAQVVAIASNNGGKQA
LETVQRLLPVLCQAHGLTPEQVVAIASNGGGKQALESIVAQLSRP
DPALAALTNDHLVALACLGGRPALDAVKKGLGGSGSAIPVKRGAT
GETKVFIGNSNSPKSPTKGGCSGGSTNLSDIIEKETGKQLVIQES
ILMLPEEVEEVIGNKPESDILVHTAYDESTDENVMLLTSDAPEYK
PWALVIQDSNGENKIKML.
(The underlined portion corresponds to the TALE
protein including the N-terminal domain and
C-terminal domain (including the half domain))
SEQ ID NO: 72
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPEQVVAIASNGGGKQALETVQRLLPVLCQAH
GLTPDQVVAIASNGGGKQALETVQRLLPVLCQDHGLTPDQVVAIA
SNIGGKQALETVQRLLPVLCQDHGLTPAQVVAIASNIGGKQALET
VQRLLPVLCQAHGLTPDQVVAIASNGGGKQALETVQRLLPVLCQA
HGLTPDQVVAIASNGGGKQALETVQRLLPVLCQAHGLTPDQVVAI
ASNGGGKQALETVQRLLPVLCQAHGLTPDQVVAIASNIGGKQALE
TVQRLLPVLCQDHGLTPDQVVAIASNGGGKQALETVQRLLPVLCQ
AHGLTPEQVVAIASNGGGKQALETVQRLLPVLCQAHGLTPDQVVA
IASNGGGKQALETVQRLLPVLCQDHGLTPDQVVAIASNIGGKQAL
ETVQRLLPVLCQDHGLTPAQVVAIASNNGGKQALETVQRLLPVLC
QAHGLTPDQVVAIASNNGGKQALETVQRLLPVLCQDHGLTPEQVV
AIASNNGGKQALETVQRLLPVLCQAHGLTPAQVVAIASNNGGKQA
LETVQRLLPVLCQAHGLTPAQVVAIASNNGGKQALETVQRLLPVL
CQAHGLTPEQVVAIASNGGGKQALESIVAQLSRPDPALAALTNDH
LVALACLGGRPALDAVKKGLGGSGSAIPVKRGATGETKVFIGNSN
SPKSPTKGGCSGGSTNLSDIIEKETGKQLVIQESILMLPEEVEEV
IGNKPESDILVHTAYDESTDENVMLLTSDAPEYKPWALVIQDSNG
ENKIKML.
(The underlined portion corresponds to the TALE
protein including the N-terminal domain and
C-terminal domain (including the half domain))
SEQ ID NO: 73
MASVLTPLLLRGLTGSARRLPVPRAKIHSLDYKDHDGDYKDHDID
YKDDDDKGIRIQDLRTLGYSQQQQEKIKPKVRSTVAQHHEALVGH
GFTHAHIVALSQHPAALGTVAVKYQDMIAALPEATHEAIVGVGKQ
WSGARALEALLTVAGELRGPPLQLDTGQLLKIAKRGGVTAVEAVH
AWRNALTGAPLNLTPEQVVAIASNGGGKQALETVQRLLPVLCQAH
GLTPDQVVAIASNGGGKQALETVQRLLPVLCQDHGLTPDQVVAIA
SNIGGKQALETVQRLLPVLCQDHGLTPAQVVAIASNIGGKQALET
VQRLLPVLCQAHGLTPDQVVAIASNGGGKQALETVQRLLPVLCQA
HGLTPDQVVAIASNGGGKQALETVQRLLPVLCQAHGLTPDQVVAI
ASNGGGKQALETVQRLLPVLCQAHGLTPDQVVAIASNIGGKQALE
TVQRLLPVLCQDHGLTPDQVVAIASNGGGKQALETVQRLLPVLCQ
AHGLTPEQVVAIASNGGGKQALETVQRLLPVLCQAHGLTPDQVVA
IASNGGGKQALETVQRLLPVLCQDHGLTPDQVVAIASNIGGKQAL
ETVQRLLPVLCQDHGLTPAQVVAIASNNGGKQALETVQRLLPVLC
QAHGLTPAQVVAIASNNGGKQALETVQRLLPVLCQAHGLTPAQVV
AIASNNGGKQALETVQRLLPVLCQAHGLTPAQVVAIASNNGGKQA
LETVQRLLPVLCQAHGLTPEQVVAIASNGGGKQALESIVAQLSRP
DPALAALTNDHLVALACLGGRPALDAVKKGLGGSGSAIPVKRGAT
GETKVFIGNSNSPKSPTKGGCSGGSTNLSDIIEKETGKQLVIQES
ILMLPEEVEEVIGNKPESDILVHTAYDESTDENVMLLTSDAPEYK
PWALVIQDSNGENKIKML.
(The underlined portion corresponds to the TALE
protein including the N-terminal domain and
C-terminal domain (including the half domain))
SEQ ID NO: 74
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPDQVVAIASNGGGKQALETVQRL
LPVLCQAHGLTPAQVVAIASNGGGKQALETVQRLLPVLCQAHGLT
PEQVVAIASNGGGKQALETVQRLLPVLCQAHGLTPEQVVAIASNG
GGKQALETVQRLLPVLCQAHGLTPAQVVAIASNIGGKQALETVQR
LLPVLCQDHGLTPAQVVAIASNIGGKQALETVQRLLPVLCQAHGL
TPDQVVAIASNGGGKQALETVQRLLPVLCQAHGLTPAQVVAIASN
GGGKQALETVQRLLPVLCQAHGLTPEQVVAIASNGGGKQALETVQ
RLLPVLCQAHGLTPAQVVAIASNIGGKQALETVQRLLPVLCQAHG
LTPDQVVAIASNGGGKQALETVQRLLPVLCQAHGLTPDQVVAIAS
NGGGKQALETVQRLLPVLCQAHGLTPAQVVAIASNGGGKQALETV
QRLLPVLCQDHGLTPAQVVAIASNIGGKQALETVQRLLPVLCQAH
GLTPDQVVAIASNNGGKQALETVQRLLPVLCQAHGLTPAQVVAIA
SNNGGKQALETVQRLLPVLCQAHGLTPDQVVAIASNNGGKQALET
VQRLLPVLCQDHGLTPEQVVAIASNNGGKQALETVQRLLPVLCQA
HGLTPEQVVAIASNGGGKQALESIVAQLSRPDPALAALTNDHLVA
LACLGGRPALDAVKKGLGGSGSGSYALGPYQISAPQLPAYNGQTV
GTFYYVNDAGGLESKVFISGGPTPYPNYVSAGHVEGQSALFMRDN
GISEGLVFHNNPKGTCGFCVNMIETLLPENAAMTVVPPEGSGGST
NLSDIIEKETGKQLVIQESILMLPEEVEEVIGNKPESDILVHTAY
DESTDENVMLLTSDAPEYKPWALVIQDSNGENKIKML.
(The underlined portion corresponds to the TALE
protein including the N-terminal domain and C-
terminal domain (including the half domain))
SEQ ID NO: 75
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPDQVVAIASNGGGKQALETVQRL
LPVLCQAHGLTPAQVVAIASNGGGKQALETVQRLLPVLCQAHGLT
PEQVVAIASNGGGKQALETVQRLLPVLCQAHGLTPEQVVAIASNG
GGKQALETVQRLLPVLCQAHGLTPAQVVAIASNIGGKQALETVQR
LLPVLCQDHGLTPAQVVAIASNIGGKQALETVQRLLPVLCQAHGL
TPDQVVAIASNGGGKQALETVQRLLPVLCQAHGLTPAQVVAIASN
GGGKQALETVQRLLPVLCQAHGLTPEQVVAIASNGGGKQALETVQ
RLLPVLCQAHGLTPAQVVAIASNIGGKQALETVQRLLPVLCQAHG
LTPDQVVAIASNGGGKQALETVQRLLPVLCQAHGLTPDQVVAIAS
NGGGKQALETVQRLLPVLCQAHGLTPAQVVAIASNGGGKQALETV
QRLLPVLCQDHGLTPAQVVAIASNIGGKQALETVQRLLPVLCQAH
GLTPDQVVAIASNNGGKQALETVQRLLPVLCQAHGLTPAQVVAIA
SNNGGKQALETVQRLLPVLCQAHGLTPAQVVAIASNNGGKQALET
VQRLLPVLCQAHGLTPEQVVAIASNGGGKQALESIVAQLSRPDPA
LAALTNDHLVALACLGGRPALDAVKKGLGGSGSGSYALGPYQISA
PQLPAYNGQTVGTFYYVNDAGGLESKVFISGGPTPYPNYVSAGHV
EGQSALFMRDNGISEGLVFHNNPKGTCGFCVNMIETLLPENAAMT
VVPPEGSGGSTNLSDIIEKETGKQLVIQESILMLPEEVEEVIGNK
PESDILVHTAYDESTDENVMLLTSDAPEYKPWALVIQDSNGENKI
KML.
(The underlined portion corresponds to the TALE
protein including the N-terminal domain and
C-terminal domain (including the half domain))
SEQ ID NO: 76
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPDQVVAIASNGGGKQALETVQRL
LPVLCQAHGLTPAQVVAIASNGGGKQALETVQRLLPVLCQAHGLT
PEQVVAIASNGGGKQALETVQRLLPVLCQAHGLTPEQVVAIASNG
GGKQALETVQRLLPVLCQAHGLTPAQVVAIASNIGGKQALETVQR
LLPVLCQDHGLTPAQVVAIASNIGGKQALETVQRLLPVLCQAHGL
TPDQVVAIASNGGGKQALETVQRLLPVLCQAHGLTPAQVVAIASN
GGGKQALETVQRLLPVLCQAHGLTPEQVVAIASNGGGKQALETVQ
RLLPVLCQAHGLTPAQVVAIASNIGGKQALETVQRLLPVLCQAHG
LTPDQVVAIASNGGGKQALETVQRLLPVLCQAHGLTPDQVVAIAS
NGGGKQALETVQRLLPVLCQAHGLTPAQVVAIASNGGGKQALETV
QRLLPVLCQDHGLTPAQVVAIASNIGGKQALETVQRLLPVLCQDH
GLTPAQVVAIASNNGGKQALETVQRLLPVLCQAHGLTPAQVVAIA
SNNGGKQALETVQRLLPVLCQAHGLTPEQVVAIASNGGGKQALES
IVAQLSRPDPALAALTNDHLVALACLGGRPALDAVKKGLGGSGSG
SYALGPYQISAPQLPAYNGQTVGTFYYVNDAGGLESKVFISGGPT
PYPNYVSAGHVEGQSALFMRDNGISEGLVFHNNPKGTCGFCVNMI
ETLLPENAAMTVVPPEGSGGSTNLSDIIEKETGKQLVIQESILML
PEEVEEVIGNKPESDILVHTAYDESTDENVMLLTSDAPEYKPWAL
VIQDSNGENKIKML.
(The underlined portion corresponds to the TALE
protein including the N-terminal domain and
C-terminal domain (including the half domain))
SEQ ID NO: 77
MALSRAVCGTSRQLAPVLGYLGSRQKHSLPDYPYDVPDYAGYPYD
VPDYAGYPYDVPDYAGIRIQDLRTLGYSQQQQEKIKPKVRSTVAQ
HHEALVGHGFTHAHIVALSQHPAALGTVAVKYQDMIAALPEATHE
AIVGVGKQWSGARALEALLTVAGELRGPPLQLDTGQLLKIAKRGG
VTAVEAVHAWRNALTGAPLNLTPDQVVAIASNGGGKQALETVQRL
LPVLCQAHGLTPAQVVAIASNGGGKQALETVQRLLPVLCQAHGLT
PEQVVAIASNGGGKQALETVQRLLPVLCQAHGLTPEQVVAIASNG
GGKQALETVQRLLPVLCQAHGLTPAQVVAIASNIGGKQALETVQR
LLPVLCQDHGLTPAQVVAIASNIGGKQALETVQRLLPVLCQAHGL
TPDQVVAIASNGGGKQALETVQRLLPVLCQAHGLTPAQVVAIASN
GGGKQALETVQRLLPVLCQAHGLTPEQVVAIASNGGGKQALETVQ
RLLPVLCQAHGLTPAQVVAIASNIGGKQALETVQRLLPVLCQAHG
LTPDQVVAIASNGGGKQALETVQRLLPVLCQAHGLTPDQVVAIAS
NGGGKQALETVQRLLPVLCQAHGLTPAQVVAIASNGGGKQALETV
QRLLPVLCQDHGLTPAQVVAIASNIGGKQALETVQRLLPVLCQDH
GLTPDQVVAIASNNGGKQALETVQRLLPVLCQAHGLTPEQVVAIA
SNGGGKQALESIVAQLSRPDPALAALTNDHLVALACLGGRPALDA
VKKGLGGSGSGSYALGPYQISAPQLPAYNGQTVGTFYYVNDAGGL
ESKVFISGGPTPYPNYVSAGHVEGQSALFMRDNGISEGLVFHNNP
KGTCGFCVNMIETLLPENAAMTVVPPEGSGGSTNLSDIIEKETGK
QLVIQESILMLPEEVEEVIGNKPESDILVHTAYDESTDENVMLLT
SDAPEYKPWALVIQDSNGENKIKML.
(The underlined portion corresponds to the TALE
protein including the N-terminal domain and
C-terminal domain (including the half domain))

The combinations of the first fusion protein and the second fusion protein used in the base editors shown in FIG. 1 are as follows.

| Base Editor ID | First Fusion Protein | Second Fusion Protein |
|---|---|---|
| 22-4-1 | SEQ ID NO: 66 | SEQ ID NO: 70 |
| 22-5-1 | SEQ ID NO: 67 | SEQ ID NO: 70 |
| 23-4-1 | SEQ ID NO: 66 | SEQ ID NO: 71 |
| 23-5-1 | SEQ ID NO: 67 | SEQ ID NO: 71 |
| 24-4-1 | SEQ ID NO: 66 | SEQ ID NO: 72 |
| 24-4-2 | SEQ ID NO: 66 | SEQ ID NO: 73 |
| 24-5-1 | SEQ ID NO: 67 | SEQ ID NO: 72 |
| 24-5-2 | SEQ ID NO: 67 | SEQ ID NO: 73 |
| 40-3-1 | SEQ ID NO: 68 | SEQ ID NO: 74 |
| 40-3-2 | SEQ ID NO: 68 | SEQ ID NO: 75 |
| 40-3-3 | SEQ ID NO: 68 | SEQ ID NO: 76 |
| 40-3-4 | SEQ ID NO: 68 | SEQ ID NO: 77 |
| 40-4-1 | SEQ ID NO: 69 | SEQ ID NO: 74 |
| 40-4-2 | SEQ ID NO: 69 | SEQ ID NO: 75 |
| 40-4-3 | SEQ ID NO: 69 | SEQ ID NO: 76 |

### Example 4: Construction of TALED for m.G3460A Mutation Correction

First and second TALED (TALE deaminase) fusion proteins were constructed such that the editing window encompassing the G3460A point mutation in mitochondrial ND1 DNA would span 1 to 20 bp. Specifically, 424 TAL effector array plasmids and expression plasmids (including CMV and T7 promoters, MTS, tag, DddAₜₒₓ 1397N or 1397C, TadA) were constructed using the Golden-Gate cloning system Competent DH5a cells (Enzynomics) Escherichia coli were transformed by heat shock at 42° C., and single colonies were cultured in LB medium at 37° C. overnight with shaking incubation Plasmid DNA was purified using the Plasmid SV mini kit (GeneAll) according to the manufacturer's protocol. Purified plasmid DNA, or TALEDs, were sequenced by Sanger sequencing (Macrogen).

The first fusion protein and the second fusion protein were linked in the order of [MTS]-[Tag]-[TALE protein]-[Linker]-[DddAₜₒₓ fragment] or [MTS]-[Tag]-[TALE protein]-[Linker]-[DddAₜₒₓ fragment]-[Linker]-[TadA8e]. The tag used was 3×HA (SEQ ID NO: 136) or 3×FLAG (SEQ ID NO: 137). The proteins used are located between the CMV promoter and the T7 promoter and terminator sequence. When the DNA sequence to which the TALE binds starts with 5′-T, the NTD (N-terminal domain) of the TALE protein or a variant thereof was used, and when the DNA sequence does not start with 5′-T (i.e., starts with 5′-A, 5′-C, or 5′-G), a variant NTD sequence was used to construct the protein.

### Example 5: Transfection of a Fusion Protein into Urine-Derived Cells of a Patient Having a G3460A Mutation in the Mitochondrial ND1 Gene

Primary cells were isolated from the urine of a patient with LHON having a G3460A point mutation in the mitochondrial ND1 gene, and were cryopreserved after being aliquoted at passage 2. A patient's UDCs (Urine-derived cells) were cultured at 37° C. in 5% CO₂ in 12-well Clear TC-Treated Multiple Well Plates (Corning) coated with 0.1% gelatin (Welgene), using a Renal Epithelial Cell Growth Medium BulletKit (REGM, Lonza). The UDCs were seeded in 96-well Clear TCTreated Multiple Well Plates (Corning) at a density of 0.6×10⁴ cells per well. A total of 500 ng of plasmids, consisting of 250 ng of a plasmid encoding the first fusion protein and 250 ng of a plasmid encoding the second fusion protein, was transfected using Lipofectamine LTX reagent (Invitrogen) and placed into the pre-seeded 96-well plates. Transfected cells were maintained in culture at 37° C. in 5% CO₂ while replacing the culture medium. After 6 days, the cells were harvested, the culture medium was removed, and 50 μL of cell lysis buffer (50 mM Tris-HCl pH 7.4 (Welgene), 1 mM EDTA pH 8.0 (Welgene), 0.005% sodium dodecyl sulfate (Welgene), 5 uL Proteinase K (Qiagen)) was added to each well, and incubated in a PCR machine at 50° C. for 1 hour and at 80° C. for 20 minutes.

### Example 6: Sequencing and Base Editing Efficiency Measurement for Confirmation of m.G3460A Mutation Correction

The reaction product obtained in Example 5 was used directly as a template without purification, and the target site was analyzed by targeted deep sequencing to evaluate the base editing efficiency. To construct a deep sequencing library, nested first PCR and second PCR using the product of the first PCR as a template were performed using PrimeSTAR® GXL DNA Polymerase (TAKARA), and a third PCR was performed using index-containing primers to add the final index sequence. The third PCR reaction product with added index sequences was purified using the PCR SV mini kit (GeneAll), and paired-end sequencing was performed using the MiniSeq system (Illumina) with the MiniSeq Mid Output Kit (Illumina).

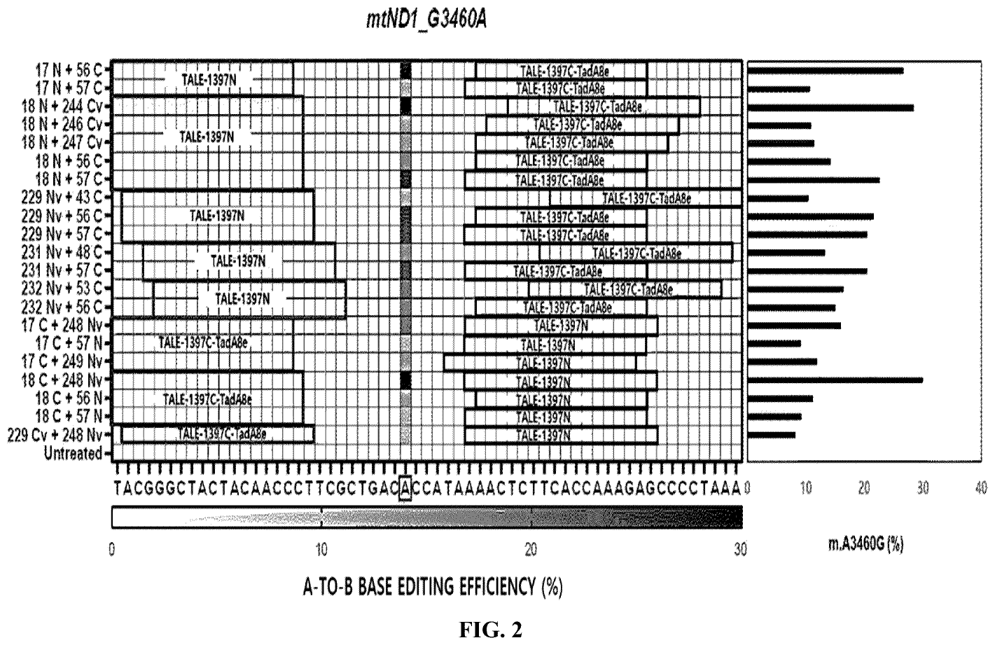

The efficiencies of m.A3460G correction of the constructed base editors were screened, and the DNA binding sites and the efficiencies of m. A3460G correction of 21 base editor combinations having high base editing efficiency are shown in FIG. 2.

The amino acid sequences of the first fusion protein (including the first TALE) and the second fusion protein (including the second TALE), which were used in the 21 base editor combinations confirmed to have high base editing efficiency, are as follows.

First fusion protein
SEQ ID NO: 78
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKRGG
VTAVEAVHAWRNALTGAPLNLTPDQVVAIASNIGGKQALETVQRLLPVLCQDHGLTPDQV
VAIASHDGGKQALETVQRLLPVLCQAHGLTPAQVVAIASNNGGKQALETVQRLLPVLCQD
HGLTPEQVVAIASNNGGKQALETVQRLLPVLCQAHGLTPAQVVAIASNNGGKQALETVQR
LLPVLCQDHGLTPDQVVAIASHDGGKQALETVQRLLPVLCQDHGLTPEQVVAIASNGGGK
QALETVQRLLPVLCQAHGLTPDQVVAIASNIGGKQALETVQRLLPVLCQAHGLTPAQVVA
IASHDGGKQALETVQRLLPVLCQAHGLTPEQVVAIASNGGGKQALETVQRLLPVLCQAHG
LTPDQVVAIASNIGGKQALETVQRLLPVLCQAHGLTPAQVVAIASHDGGKQALETVQRLL
PVLCQDHGLTPDQVVAIASNIGGKQALETVQRLLPVLCQDHGLTPAQVVAIASNIGGKQA
LETVQRLLPVLCQAHGLTPAQVVAIASHDGGKQALETVQRLLPVLCQAHGLTPEQVVAIA
SNGGGKQALESIVAQLSRPDPALAALTNDHLVALACLGGRPALDAVKKGLGGSGSGSYAL
GPYQISAPQLPAYNGQTVGTFYYVNDAGGLESKVFSSGGPTPYPNYANAGHVEGQSALFM
RDNGISEGLVFHNNPEGTCGFCVNMTETLLPENAKMTVVPPEG.
(The underlined portion corresponds to the TALE protein
including the N-terminal domain and the C-terminal domain
(including the half domain))
SEQ ID NO: 79
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKRGG
VTAVEAVHAWRNALTGAPLNLTPDQVVAIASNIGGKQALETVQRLLPVLCQDHGLTPDQV
VAIASHDGGKQALETVQRLLPVLCQAHGLTPAQVVAIASNNGGKQALETVQRLLPVLCQD
HGLTPEQVVAIASNNGGKQALETVQRLLPVLCQAHGLTPAQVVAIASNNGGKQALETVQR
LLPVLCQDHGLTPDQVVAIASHDGGKQALETVQRLLPVLCQDHGLTPEQVVAIASNGGGK
QALETVQRLLPVLCQAHGLTPDQVVAIASNIGGKQALETVQRLLPVLCQAHGLTPAQVVA
IASHDGGKQALETVQRLLPVLCQAHGLTPEQVVAIASNGGGKQALETVQRLLPVLCQAHG
LTPDQVVAIASNIGGKQALETVQRLLPVLCQAHGLTPAQVVAIASHDGGKQALETVQRLL
PVLCQDHGLTPDQVVAIASNIGGKQALETVQRLLPVLCQDHGLTPAQVVAIASNIGGKQA
LETVQRLLPVLCQAHGLTPAQVVAIASHDGGKQALETVQRLLPVLCQDHGLTPAQVVAIA
SHDGGKQALETVQRLLPVLCQAHGLTPEQVVAIASNGGGKQALESIVAQLSRPDPALAAL
TNDHLVALACLGGRPALDAVKKGLGGSGSGSYALGPYQISAPQLPAYNGQTVGTFYYVND
AGGLESKVFSSGGPTPYPNYANAGHVEGQSALFMRDNGISEGLVFHNNPEGTCGFCVNMT
ETLLPENAKMTVVPPEG.
(The underlined portion corresponds to the TALE
protein including the N-terminal domain and
the C-terminal domain (including the half domain))
SEQ ID NO: 80
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPEQVVAIASHDGGKQALETVQRLLPVLCQAHGLTPAQVV
AIASNNGGKQALETVQRLLPVLCQDHGLTPDQVVAIASNNGGKQALETVQRLLPVLCQDH
GLTPDQVVAIASNNGGKQALETVQRLLPVLCQAHGLTPAQVVAIASHDGGKQALETVQRL
LPVLCQDHGLTPDQVVAIASNGGGKQALETVQRLLPVLCQAHGLTPEQVVAIASNIGGKQ
ALETVQRLLPVLCQAHGLTPDQVVAIASHDGGKQALETVQRLLPVLCQAHGLTPDQVVAI
ASNGGGKQALETVQRLLPVLCQAHGLTPDQVVAIASNIGGKQALETVQRLLPVLCQDHGL
TPDQVVAIASHDGGKQALETVQRLLPVLCQDHGLTPAQVVAIASNIGGKQALETVQRLLP
VLCQAHGLTPEQVVAIASNIGGKQALETVQRLLPVLCQAHGLTPAQVVAIASHDGGKQAL
ETVQRLLPVLCQDHGLTPAQVVAIASHDGGKQALETVQRLLPVLCQDHGLTPAQVVAIAS
HDGGKQALETVQRLLPVLCQAHGLTPEQVVAIASNGGGKQALESIVAQLSRPDPALAALT
NDHLVALACLGGRPALDAVKKGLGGSGSGSYALGPYQISAPQLPAYNGQTVGTFYYVNDA
GGLESKVFSSGGPTPYPNYANAGHVEGQSALFMRDNGISEGLVFHNNPEGTCGFCVNMTE
TLLPENAKMTVVPPEG.
(The underlined portion corresponds to the TALE
protein including the N-terminal domain and
the C-terminal domain (including the half domain))
SEQ ID NO: 81
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPEQVVAIASNNGGKQALETVQRLLPVLCQAHGLTPAQVV
AIASNNGGKQALETVQRLLPVLCQDHGLTPDQVVAIASHDGGKQALETVQRLLPVLCQDH
GLTPEQVVAIASNGGGKQALETVQRLLPVLCQAHGLTPDQVVAIASNIGGKQALETVQRL
LPVLCQAHGLTPAQVVAIASHDGGKQALETVQRLLPVLCQDHGLTPEQVVAIASNGGGKQ
ALETVQRLLPVLCQAHGLTPDQVVAIASNIGGKQALETVQRLLPVLCQAHGLTPAQVVAI
ASHDGGKQALETVQRLLPVLCQAHGLTPDQVVAIASNIGGKQALETVQRLLPVLCQAHGL
TPDQVVAIASNIGGKQALETVQRLLPVLCQAHGLTPAQVVAIASHDGGKQALETVQRLLP
VLCQAHGLTPAQVVAIASHDGGKQALETVQRLLPVLCQDHGLTPAQVVAIASHDGGKQAL
ETVQRLLPVLCQAHGLTPEQVVAIASNGGGKQALETVQRLLPVLCQAHGLIPDQVVAIAS
NGGGKQALETVQRLLPVLCQAHGLTPEQVVAIASNGGGKQALESIVAQLSRPDPALAALI
NDHLVALACLGGRPALDAVKKGLGGSGSGSYALGPYQISAPQLPAYNGQTVGTFYYVNDA
GGLESKVFSSGGPTPYPNYANAGHVEGQSALFMRDNGISEGLVFHNNPEGTCGFCVNMTE
TLLPENAKMTVVPPEG.
(The underlined portion corresponds to the TALE
protein including the N-terminal domain
and the C-terminal domain (including the half domain))
SEQ ID NO: 82
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGETHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPDQVVAIASNNGGKQALETVQRLLPVLCQAHGLTPAQVV
AIASHDGGKQALETVQRLLPVLCQDHGLIPDQVVAIASNGGGKQALETVQRLLPVLCQAH
GLTPEQVVAIASNIGGKQALETVQRLLPVLCQAHGLTPDQVVAIASHDGGKQALETVQRL
LPVLCQAHGLTPDQVVAIASNGGGKQALETVQRLLPVLCQDHGLTPDQVVAIASNIGGKQ
ALETVQRLLPVLCQDHGLTPDQVVAIASHDGGKQALETVQRLLPVLCQDHGLTPAQVVAI
ASNIGGKQALETVQRLLPVLCQAHGLTPEQVVAIASNIGGKQALETVQRLLPVLCQAHGL
TPAQVVAIASHDGGKQALETVQRLLPVLCQAHGLIPAQVVAIASHDGGKQALETVQRLLP
VLCQDHGLTPDQVVAIASHDGGKQALETVQRLLPVLCQAHGLTPAQVVAIASNGGGKQAL
ETVQRLLPVLCQDHGLTPAQVVAIASNGGGKQALETVQRLLPVLCQDHGLTPEQVVAIAS
HDGGKQALETVQRLLPVLCQAHGLTPEQVVAIASNGGGKQALESIVAQLSRPDPALAALT
NDHLVALACLGGRPALDAVKKGLGGSGSGSYALGPYQISAPQLPAYNGQTVGTFYYVNDA
GGLESKVFSSGGPTPYPNYANAGHVEGQSALFMRDNGISEGLVFHNNPEGTCGFCVNMTE
TLLPENAKMTVVPPEG.
(The underlined portion corresponds to the TALE
protein including the N-terminal domain and
the C-terminal domain (including the half domain))
SEQ ID NO: 83
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPDQVVAIASNIGGKQALETVQRLLPVLCQDHGLTPDQVVAIASHDG
GKQALETVQRLLPVLCQAHGLTPAQVVAIASNNGGKQALETVQRLLPVLCQDHGLTPEQV
VAIASNNGGKQALETVQRLLPVLCQAHGLTPAQVVAIASNNGGKQALETVQRLLPVLCQD
HGLTPDQVVAIASHDGGKQALETVQRLLPVLCQDHGLTPEQVVAIASNGGGKQALETVQR
LLPVLCQAHGLTPDQVVAIASNIGGKQALETVQRLLPVLCQAHGLTPAQVVAIASHDGGK
QALETVQRLLPVLCQAHGLTPEQVVAIASNGGGKQALETVQRLLPVLCQAHGLTPDQVVA
IASNIGGKQALETVQRLLPVLCQAHGLTPAQVVAIASHDGGKQALETVQRLLPVLCQDHG
LTPDQVVAIASNIGGKQALETVQRLLPVLCQDHGLTPAQVVAIASNIGGKQALETVQRLL
PVLCQAHGLTPAQVVAIASHDGGKQALETVQRLLPVLCQAHGLTPEQVVAIASNGGGKQA
LESIVAQLSRPDPALAALINDHLVALACLGGRPALDAVKKGLLVGSAIPVKRGATGETKV
FTGNSNSPKSPTKGGCSGSETPGTSESATPESSEVEFSHEYWMRHALTLAKRARDEREVP
VGAVLVLNNRVIGEGWNRAIGLHDPTAHAEIMALRQGGLVMQNYRLIDATLYVTFEPCVM
CAGAMIHSRIGRVVFGVRNSKRGAAGSLMNVLNYPGMNHRVEITEGILADECAALLCDFY
RMPRQVENAQKKAQSSIN.
(The underlined portion corresponds to the TALE
protein including the N-terminal
domain and the C-terminal domain (including the half domain))
SEQ ID NO: 84
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPDQVVAIASNIGGKQALETVQRLLPVLCQDHGLTPDQVVAIASHDG
GKQALETVQRLLPVLCQAHGLTPAQVVAIASNNGGKQALETVQRLLPVLCQDHGLTPEQV
VAIASNNGGKQALETVQRLLPVLCQAHGLTPAQVVAIASNNGGKQALETVQRLLPVLCQD
HGLTPDQVVAIASHDGGKQALETVQRLLPVLCQDHGLTPEQVVAIASNGGGKQALETVQR
LLPVLCQAHGLTPDQVVAIASNIGGKQALETVQRLLPVLCQAHGLTPAQVVAIASHDGGK
QALETVQRLLPVLCQAHGLTPEQVVAIASNGGGKQALETVQRLLPVLCQAHGLIPDQVVA
IASNIGGKQALETVQRLLPVLCQAHGLTPAQVVAIASHDGGKQALETVQRLLPVLCQDHG
LTPDQVVAIASNIGGKQALETVQRLLPVLCQDHGLTPAQVVAIASNIGGKQALETVQRLL
PVLCQAHGLTPAQVVAIASHDGGKQALETVQRLLPVLCQDHGLTPAQVVAIASHDGGKQA
LETVQRLLPVLCQAHGLTPEQVVAIASNGGGKQALESIVAQLSRPDPALAALINDHLVAL
ACLGGRPALDAVKKGLLVGSAIPVKRGATGETKVFTGNSNSPKSPTKGGCSGSETPGTSE
SATPESSEVEFSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFGVRNSKRGAAG
SLMNVLNYPGMNHRVEITEGILADECAALLCDFYRMPRQVFNAQKKAQSSIN.
(The underlined portion
corresponds to the TALE
protein including the N-terminal domain and the C-terminal
domain (including the half domain))
SEQ ID NO: 85
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPEQVVAIASHDGGKQALETVQRLLPVLCQAHGLTPAQVVAIASNNGG
KQALETVQRLLPVLCQDHGLTPDQVVAIASNNGGKQALETVQRLLPVLCQDHGLIPDQVV
AIASNNGGKQALETVQRLLPVLCQAHGLTPAQVVAIASHDGGKQALETVQRLLPVLCQDH
GLTPDQVVAIASNGGGKQALETVQRLLPVLCQAHGLTPEQVVAIASNIGGKQALETVQRL
LPVLCQAHGLTPDQVVAIASHDGGKQALETVQRLLPVLCQAHGLTPDQVVAIASNGGGKQ
ALETVQRLLPVLCQAHGLTPDQVVAIASNIGGKQALETVQRLLPVLCQDHGLTPDQVVAI
ASHDGGKQALETVQRLLPVLCQDHGLTPAQVVAIASNIGGKQALETVQRLLPVLCQAHGL
IPEQVVAIASNIGGKQALETVQRLLPVLCQAHGLTPAQVVAIASHDGGKQALETVQRLLP
VLCQDHGLTPAQVVAIASHDGGKQALETVQRLLPVLCQDHGLTPAQVVAIASHDGGKQAL
ETVQRLLPVLCQAHGLTPEQVVAIASNGGGKQALESIVAQLSRPDPALAALINDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HABEIMALRQGGLVMQNYRLIDATLYVTFEPCVMCAGAMIHSRIGRVVFGVRNSKRGAAG
SLMNVLNYPGMNHRVEITEGILADECAALLCDFYRMPRQVFNAQKKAQSSIN.
(The underlined portion
corresponds to the TALE
protein including the N-terminal domain and the C-terminal
domain (including the half domain))
Second fusion protein
SEQ ID NO: 86
MALSRAVCGTSRQLAPVLGYLGSRQKHSLPDYPYDYPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKRGG
VTAVEAVHAWRNALTGAPLNLTPAQVVAIASHDGGKQALETVQRLLPVLCQAHGLTPDQV
VAIASNGGGKQALETVQRLLPVLCQAHGLTPAQVVAIASNGGGKQALETVQRLLPVLCQD
HGLTPEQVVAIASNGGGKQALETVQRLLPVLCQAHGLTPAQVVAIASNNGGKQALETVQR
LLPVLCQDHGLTPDQVVAIASNNGGKQALETVQRLLPVLCQDHGLIPAQVVAIASNGGGK
QALETVQRLLPVLCQAHGLTPAQVVAIASNNGGKQALETVQRLLPVLCQDHGLTPAQVVA
IASNIGGKQALETVQRLLPVLCQAHGLIPDQVVAIASNIGGKQALETVQRLLPVLCQDHG
LTPAQVVAIASNNGGKQALETVQRLLPVLCQDHGLTPAQVVAIASNIGGKQALETVQRLL
PVLCQAHGLTPDQVVAIASNNGGKQALETVQRLLPVLCQAHGLTPAQVVAIASNGGGKQA
LETVQRLLPVLCQAHGLTPEQVVAIASNGGGKQALESIVAQLSRPDPALAALINDHLVAL
ACLGGRPALDAVKKGLGGSGSGSYALGPYQISAPQLPAYNGQTVGTFYYVNDAGGLESKV
FSSGGPTPYPNYANAGHVEGQSALEMRDNGISEGLVFHNNPEGTCGFCVNMTETLLPENA
KMTVVPPEG.
(The underlined portion
corresponds to the TALE
protein including the N-terminal domain and the C-terminal
domain (including the half domain))
SEQ ID NO: 87
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKRGG
VTAVEAVHAWRNALTGAPLNLTPAQVVAIASHDGGKQALETVQRLLPVLCQAHGLTPDQV
VAIASNGGGKQALETVQRLLPVLCQAHGLTPAQVVAIASNGGGKQALETVQRLLPVLCQD
HGLTPEQVVAIASNGGGKQALETVQRLLPVLCQAHGLTPAQVVAIASNNGGKQALETVQR
LLPVLCQDHGLTPDQVVAIASNNGGKQALETVQRLLPVLCQDHGLTPAQVVAIASNGGGK
QALETVQRLLPVLCQAHGLTPAQVVAIASNNGGKQALETVQRLLPVLCQDHGLTPAQVVA
IASNIGGKQALETVQRLLPVLCQAHGLTPDQVVAIASNIGGKQALETVQRLLPVLCQDHG
LTPAQVVAIASNNGGKQALETVQRLLPVLCQDHGLTPAQVVAIASNIGGKQALETVQRLL
PVLCQAHGLTPDQVVAIASNNGGKQALETVQRLLPVLCQAHGLTPDQVVAIASNGGGKQA
LETVQRLLPVLCQAHGLTPAQVVAIASNGGGKQALETVQRLLPVLCQAHGLTPEQVVAIA
SNGGGKQALESIVAQLSRPDPALAALINDHLVALACLGGRPALDAVKKGLGGSGSGSYAL
GPYQISAPQLPAYNGQTVGTFYYVNDAGGLESKVFSSGGPTPYPNYANAGHVEGQSALFM
RDNGISEGLVFHNNPEGTCGFCVNMTETLLPENAKMTVVPPEG.
(The underlined portion corresponds to the TALE protein
including the N-terminal domain and the C-terminal domain
(including the half domain))
SEQ ID NO: 88
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPEQVVAIASNGGGKQALETVQRLLPVLCQAHGLTPDQVV
AIASHDGGKQALETVQRLLPVLCQDHGLTPDQVVAIASNGGGKQALETVQRLLPVLCQAH
GLTPEQVVAIASNGGGKQALETVQRLLPVLCQAHGLTPAQVVAIASNGGGKQALETVQRL
LPVLCQAHGLTPDQVVAIASNNGGKQALETVQRLLPVLCQDHGLTPEQVVAIASNNGGKQ
ALETVQRLLPVLCQAHGLTPDQVVAIASNGGGKQALETVQRLLPVLCQDHGLTPAQVVAI
ASNNGGKQALETVQRLLPVLCQAHGLTPAQVVAIASNIGGKQALETVQRLLPVLCQDHGL
TPDQVVAIASNIGGKQALETVQRLLPVLCQDHGLTPDQVVAIASNNGGKQALETVQRLLP
VLCQAHGLTPDQVVAIASNIGGKQALETVQRLLPVLCQDHGLTPAQVVAIASNNGGKQAL
ETVQRLLPVLCQDHGLTPEQVVAIASNGGGKQALETVQRLLPVLCQAHGLIPDQVVAIAS
NGGGKQALETVQRLLPVLCQAHGLTPEQVVAIASNGGGKQALESIVAQLSRPDPALAALT
NDHLVALACLGGRPALDAVKKGLGGSGSGSYALGPYQISAPQLPAYNGQTVGTFYYVNDA
GGLESKVFSSGGPTPYPNYANAGHVEGQSALFMRDNGISEGLVFHNNPEGTCGFCVNMTE
TLLPENAKMTVVPPEG.
(The underlined portion corresponds to the TALE
protein including the N-terminal domain
and the C-terminal domain (including the half domain))
SEQ ID NO: 89
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGETHAHIVALSQHPAALGTVAV
KYQDMIAALPEATHEAIVGVGKYHGARALEALLTVAGELRGPPLQLDTGQLLKIAKRGGY
TAVEAVHAWRNALTGAPLNLTPDQVVAIASNGGGKQALETVQRLLPVLCQAHGLTPAQVV
AIASNGGGKQALETVQRLLPVLCQAHGLIPEQVVAIASNGGGKQALETVQRLLPVLCQAH
GLTPAQVVAIASNNGGKQALETVQRLLPVLCQDHGLTPAQVVAIASNNGGKQALETVQRL
LPVLCQDHGLTPDQVVAIASNGGGKQALETVQRLLPVLCQAHGLTPEQVVAIASNNGGKQ
ALETVQRLLPVLCQAHGLTPDQVVAIASNIGGKQALETVQRLLPVLCQDHGLTPDQVVAI
ASNIGGKQALETVQRLLPVLCQAHGLTPDQVVAIASNNGGKQALETVQRLLPVLCQDHGL
TPAQVVAIASNIGGKQALETVQRLLPVLCQAHGLTPEQVVAIASNNGGKQALETVQRLLP
VLCQAHGLTPEQVVAIASNGGGKQALETVQRLLPVLCQAHGLTPDQVVAIASNGGGKQAL
ETVQRLLPVLCQAHGLTPEQVVAIASNGGGKQALETVQRLLPVLCQAHGLTPDQVVAIAS
NGGGKQALETVQRLLPVLCQAHGLTPEQVVAIASNGGGKQALESIVAQLSRPDPALAALT
NDHLVALACLGGRPALDAVKKGLGGSGSGSYALGPYQISAPQLPAYNGQTVGTFYYVNDA
GGLESKVFSSGGPTPYPNYANAGHVEGQSALFMRDNGISEGLVFHNNPEGTCGFCVNMTE
TLLPENAKMTVVPPEG.
(The underlined portion corresponds to the TALE
protein including the N-terminal domain
and the C-terminal domain (including the half domain))
SEQ ID NO: 90
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASHDGGKQALETVQRLLPVLCQAHGLIPDQVVAIASNGG
GKQALETVQRLLPVLCQAHGLTPAQVVAIASNGGGKQALETVQRLLPVLCQDHGLTPEQV
VAIASNGGGKQALETVQRNNGGKQALETVQRLLPVLCQDHGLTPAQVVAIASNGGGKQAL
ETVQRLLPVLCQAHGLTPAQVVAIASNNGGKQALETVQRLLPVLCQDHGLTPAQVVAIAS
NIGGKQALETVQRLLPVLCQAHGLTPDQVVAIASNIGGKQALETVQRLLPVLCQDHGLTP
AQVVAIASNNGGKQALETVQRLLPVLCQDHGLTPAQVVAIASNIGGKQALETVQRLLPVL
CQAHGLTPDQVVAIASNNGGKQALETVQRLLPVLCQAHGLTPAQVVAIASNGGGKQALET
VQRLLPVLCQAHGLTPEQVVAIASNGGGKQALESIVAQLSRPDPALAALTNDHLVALACL
GGRPALDAVKKGLLVGSAIPVKRGATGETKVFTGNSNSPKSPTKGGCSGSETPGTSESAT
PESSEVEFSHEYWMRHALTLAKRARDEREVPVGAVLVLNNRVIGEGWNRAIGLHDPTAHA
EIMALRQGGLVMQNYRLIDATLYVTFEPCVMCAGAMIHSRIGRVVFGVRNSKRGAAGSLM
NVLNYPGMNHRVEITEGILADECAALLCDFYRMPRQVFNAQKKAQSSIN.
(The underlined portion corresponds to the TALE
protein including the N-terminal domain and the
C-terminal domain (including the
half domain))
SEQ ID NO: 91
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASHDGGKQALETVQRLLPVLCQAHGLTPDQVVAIASNGG
GKQALETVQRLLPVLCQAHGLTPAQVVAIASNGGGKQALETVQRLLPVLCQDHGLTPEQV
VAIASNGGGKQALETVQRLLPVLCQAHGLTPAQVVAIASNNGGKQALETVQRLLPVLCQD
HGLTPDQVVAIASNNGGKQALETVQRLLPVLCQDHGLTPAQVVAIASNGGGKQALETVQR
LLPVLCQAHGLIPAQVVAIASNNGGKQALETVQRLLPVLCQDHGLTPAQVVAIASNIGGK
QALETVQRLLPVLCQAHGLTPDQVVAIASNIGGKQALETVQRLLPVLCQDHGLTPAQVVA
IASNNGGKQALETVQRLLPVLCQDHGLTPAQVVAIASNIGGKQALETVQRLLPVLCQAHG
LTPDQVVAIASNNGGKQALETVQRLLPVLCQAHGLIPDQVVAIASNGGGKQALETVQRLL
PVLCQAHGLTPAQVVAIASNGGGKQALETVQRLLPVLCQAHGLTPEQVVAIASNGGGKQA
LESIVAQLSRPDPALAALTINDHLVALACLGGRPALDAVKKGLLVGSAIPVKRGATGETK
VFTGNSNSPKSPTKGGCSGSETPGTSESATPESSEVEFSHEYWMRHALTLAKRARDEREV
PVGAVLVLNNRVIGEGWNRAIGLHDPTAHAEIMALRQGGLVMQNYRLIDATLYVTFEPCV
MCAGAMIHSRIGRVVFGVRNSKRGAAGSLMNVLNYPGMNHRVEITEGILADECAALLCDF
YRMPRQVFNAQKKAQSSIN.
(The underlined portion corresponds to the TALE
protein including the N-terminal
domain and the C-terminal domain (including the half domain))
SEQ ID NO: 92
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNNGGKQALETVQRLLPVLCQAHGLTPDQVVAIASNNGG
KQALETVQRLLPVLCQDHGLTPEQVVAIASNNGGKQALETVQRLLPVLCQAHGLTPAQVV
AIASHDGGKQALETVQRLLPVLCQDHGLTPAQVVAIASNGGGKQALETVQRLLPVLCQDH
GLTPAQVVAIASHDGGKQALETVQRLLPVLCQAHGLTPDQVVAIASNGGGKQALETVQRL
LPVLCQAHGLTPAQVVAIASNGGGKQALETVQRLLPVLCQAHGLTPEQVVAIASNGGGKQ
ALETVQRLLPVLCQAHGLTPAQVVAIASNNGGKQALETVQRLLPVLCQDHGLTPAQVVAI
ASNNGGKQALETVQRLLPVLCQDHGLTPDQVVAIASNGGGKQALETVQRLLPVLCQAHGL
TPAQVVAIASNNGGKQALETVQRLLPVLCQAHGLTPAQVVAIASNIGGKQALETVQRLLP
VLCQAHGLTPDQVVAIASNIGGKQALETVQRLLPVLCQDHGLTPAQVVAIASNNGGKQAL
ETVQRLLPVLCQDHGLTPEQVVAIASNGGGKQALESIVAQLSRPDPALAALINDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRGAAGS
LMNVLNYPGMNHRVEITEGILADECAALLCDFYRMPRQVFNAQKKAQSSIN.
(The underlined portion corresponds to the TALE
protein including the N-terminal domain and the C-terminal
domain (including the half domain))
SEQ ID NO: 93
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPDQVVAIASNNGGKQALETVQRLLPVLCQAHGLTPAQVVAIASHDGG
KQALETVQRLLPVLCQDHGLTPDQVVAIASNGGGKQALETVQRLLPVLCQAHGLTPAQVV
AIASHDGGKQALETVQRLLPVLCQAHGLTPDQVVAIASNGGGKQALETVQRLLPVLCQAH
GLTPAQVVAIASNGGGKQALETVQRLLPVLCQDHGLTPEQVVAIASNGGGKQALETVQRL
LPVLCQAHGLTPAQVVAIASNNGGKQALETVQRLLPVLCQDHGLTPDQVVAIASNNGGKQ
ALETVQRLLPVLCQAHGLTPAQVVAIASNGGGKQALETVQRLLPVLCQAHGLTPAQVVAI
ASNNGGKQALETVQRLLPVLCQDHGLTPAQVVAIASNIGGKQALETVQRLLPVLCQDHGL
TPDQVVAIASNIGGKQALETVQRLLPVLCQDHGLTPAQVVAIASNNGGKQALETVQRLLP
VLCQDHGLTPDQVVAIASNIGGKQALETVQRLLPVLCQDHGLTPAQVVAIASNNGGKQAL
ETVQRLLPVLCQDHGLTPEQVVAIASNGGGKQALESIVAQLSRPDPALAALINDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRGAAGS
LMNVLNYPGMNHRVEITEGILADECAALLCDFYRMPRQVFNAQKKAQSSIN.
(The underlined portion corresponds to the TALE
protein including the N-terminal domain and the C-terminal
domain (including the half domain))
SEQ ID NO: 94
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASHDGGKQALETVQRLLPVLCQDHGLTPAQVVAIASNGGG
KQALETVQRLLPVLCQDHGLTPAQVVAIASHDGGKQALETVQRLLPVLCQAHGLTPDQVV
AIASNGGGKQALETVQRLLPVLCQAHGLTPAQVVAIASNGGGKQALETVQRLLPVLCQAH
GLTPEQVVAIASNGGGKQALETVQRLLPVLCQAHGLTPAQVVAIASNNGGKQALETVQRL
LPVLCQDHGLTPAQVVAIASNNGGKQALETVQRLLPVLCQDHGLTPDQVVAIASNGGGKQ
ALETVQRLLPVLCQAHGLTPEQVVAIASNNGGKQALETVQRLLPVLCQAHGLTPDQVVAI
ASNIGGKQALETVQRLLPVLCQDHGLTPDQVVAIASNIGGKQALETVQRLLPVLCQAHGL
TPAQVVAIASNNGGKQALETVQRLLPVLCQAHGLTPAQVVAIASNIGGKQALETVQRLLP
VLCQAHGLTPDQVVAIASNNGGKQALETVQRLLPVLCQAHGLIPDQVVAIASNGGGKQAL
ETVQRLLPVLCQAHGLTPEQVVAIASNGGGKQALESIVAQLSRPDPALAALINDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRGAAGS
LMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))
SEQ ID NO: 95
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPEQVVAIASNGGGKQALETVQRLLPVLCQAHGLTPDQVVAIASNGG
GKQALETVQRLLPVLCQDHGLTPDQVVAIASNIGGKQALETVQRLLPVLCQDHGLTPAQV
VAIASNNGGKQALETVQRLLPVLCQAHGLTPDQVVAIASNNGGKQALETVQRLLPVLCQD
HGLTPEQVVAIASNNGGKQALETVQRLLPVLCQAHGLTPDQVVAIASNNGGKQALETVQR
LLPVLCQAHGLTPAQVVAIASHDGGKQALETVQRLLPVLCQDHGLTPDQVVAIASNGGGK
QALETVQRLLPVLCQAHGLTPAQVVAIASHDGGKQALETVQRLLPVLCQAHGLTPDQVVA
IASNGGGKQALETVQRLLPVLCQAHGLTPAQVVAIASNGGGKQALETVQRLLPVLCQDHG
LTPEQVVAIASNGGGKQALETVQRLLPVLCQAHGLTPAQVVAIASNNGGKQALETVQRLL
PVLCQDHGLTPDQVVAIASNNGGKQALETVQRLLPVLCQAHGLTPDQVVAIASNGGGKQA
LETVQRLLPVLCQAHGLTPEQVVAIASNGGGKQALESIVAQLSRPDPALAALTINDHLVA
LACLGGRPALDAVKKGLLVGSAIPVKRGATGETKVFTGNSNSPKSPTKGGCSGSETPGTS
ESATPESSEVEFSHEYWMRHALTLAKRARDEREVPVGAVLVLNNRVIGEGWNRAIGLHDP
TAHAEIMALRQGGLVMQNYRLIDATLYVTFEPCVMCAGAMIHSRIGRVVFGVRNSKRGAA
GSLMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))
SEQ ID NO: 96
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASNGGGKQALETVQRLLPVLCQDHGLTPAQVVAIASNIG
GKQALETVQRLLPVLCQAHGLTPDQVVAIASNNGGKQALETVQRLLPVLCQAHGLTPAQV
VAIASNNGGKQALETVQRLLPVLCQAHGLTPDQVVAIASNNGGKQALETVQRLLPVLCQD
HGLTPEQVVAIASNNGGKQALETVQRLLPVLCQAHGLTPAQVVAIASHDGGKQALETVQR
LLPVLCQDHGLTPAQVVAIASNGGGKQALETVQRLLPVLCQDHGLTPAQVVAIASHDGGK
QALETVQRLLPVLCQAHGLTPDQVVAIASNGGGKQALETVQRLLPVLCQAHGLTPAQVVA
IASNGGGKQALETVQRLLPVLCQAHGLTPEQVVAIASNGGGKQALETVQRLLPVLCQAHG
LTPAQVVAIASNNGGKQALETVQRLLPVLCQAHGLTPAQVVAIASNNGGKQALETVQRLL
PVLCQAHGLTPEQVVAIASNGGGKQALETVQRLLPVLCQAHGLTPAQVVAIASNNGGKQA
LETVQRLLPVLCQDHGLTPEQVVAIASNGGGKQALESIVAQLSRPDPALAALTNDHLVAL
ACLGGRPALDAVKKGLLVGSAIPVKRGATGETKVFTGNSNSPKSPTKGGCSGSETPGTSE
SATPESSEVEFSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFGVRNSKRGAAG
SLMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))
SEQ ID NO: 97
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASNIGGKQALETVQRLLPVLCQAHGLTPAQVVAIASNNG
GKQALETVQRLLPVLCQDHGLTPAQVVAIASNNGGKQALETVQRLLPVLCQAHGLTPEQV
VAIASNNGGKQALETVQRLLPVLCQAHGLTPAQVVAIASNNGGKQALETVQRLLPVLCQD
HGLTPDQVVAIASHDGGKQALETVQRLLPVLCQDHGLTPEQVVAIASNGGGKQALETVQR
LLPVLCQAHGLTPDQVVAIASHDGGKQALETVQRLLPVLCQDHGLTPDQVVAIASNGGGK
QALETVQRLLPVLCQAHGLTPEQVVAIASNGGGKQALETVQRLLPVLCQAHGLTPAQVVA
IASNGGGKQALETVQRLLPVLCQAHGLTPDQVVAIASNNGGKQALETVQRLLPVLCQDHG
LTPDQVVAIASNNGGKQALETVQRLLPVLCQAHGLTPDQVVAIASNGGGKQALETVQRLL
PVLCQAHGLTPAQVVAIASNNGGKQALETVQRLLPVLCQAHGLTPAQVVAIASNIGGKQA
LETVQRLLPVLCQAHGLTPEQVVAIASNGGGKQALESIVAQLSRPDPALAALTNDHLVAL
ACLGGRPALDAVKKGLLVGSAIPVKRGATGETKVFTGNSNSPKSPTKGGCSGSETPGTSE
SATPESSEVEFSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFGVRNSKRGAAG
SLMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))
SEQ ID NO: 98
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPEQVVAIASNGGGKQALETVQRLLPVLCQAHGLTPDQVVAIASHDGG
KQALETVQRLLPVLCQDHGLTPDQVVAIASNGGGKQALETVQRLLPVLCQAHGLTPEQVV
AIASNGGGKQALETVQRLLPVLCQAHGLTPAQVVAIASNGGGKQALETVQRLLPVLCQAH
GLTPDQVVAIASNNGGKQALETVQRLLPVLCQDHGLTPEQVVAIASNNGGKQALETVQRL
LPVLCQAHGLTPDQVVAIASNGGGKQALETVQRLLPVLCQDHGLTPAQVVAIASNNGGKQ
ALETVQRLLPVLCQAHGLTPAQVVAIASNIGGKQALETVQRLLPVLCQDHGLTPDQVVAI
ASNIGGKQALETVQRLLPVLCQDHGLTPDQVVAIASNNGGKQALETVQRLLPVLCQAHGL
TPDQVVAIASNIGGKQALETVQRLLPVLCQDHGLTPAQVVAIASNNGGKQALETVQRLLP
VLCQDHGLTPEQVVAIASNGGGKQALETVQRLLPVLCQAHGLTPDQVVAIASNGGGKQAL
ETVQRLLPVLCQAHGLTPEQVVAIASNGGGKQALESIVAQLSRPDPALAALINDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRGAAGS
LMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))
SEQ ID NO: 99
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPDQVVAIASNGGGKQALETVQRLLPVLCQAHGLTPAQVVAIASNGGG
KQALETVQRLLPVLCQAHGLTPEQVVAIASNGGGKQALETVQRLLPVLCQAHGLTPAQVV
AIASNNGGKQALETVQRLLPVLCQDHGLTPAQVVAIASNNGGKQALETVQRLLPVLCQDH
GLTPDQVVAIASNGGGKQALETVQRLLPVLCQAHGLTPEQVVAIASNNGGKQALETVQRL
LPVLCQAHGLTPDQVVAIASNIGGKQALETVQRLLPVLCQDHGLTPDQVVAIASNIGGKQ
ALETVQRLLPVLCQAHGLTPDQVVAIASNNGGKQALETVQRLLPVLCQDHGLTPAQVVAI
ASNIGGKQALETVQRLLPVLCQAHGLTPEQVVAIASNNGGKQALETVQRLLPVLCQAHGL
TPEQVVAIASNGGGKQALETVQRLLPVLCQAHGLTPDQVVAIASNGGGKQALETVQRLLP
VLCQAHGLTPEQVVAIASNGGGKQALETVQRLLPVLCQAHGLTPDQVVAIASNGGGKQAL
ETVQRLLPVLCQAHGLTPEQVVAIASNGGGKQALESIVAQLSRPDPALAALTNDHLVALA
CLGGRPALDAVKKGLLVGSAIPVKRGATGETKVFTGNSNSPKSPTKGGCSGSETPGTSES
ATPESSEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRGAAGS
LMNVLNYPGMNHRVEITEGILADECAALLCDFYRMPRQVFNAQKKAQSSIN.
(The underlined portion corresponds to the TALE
protein including the N-terminal domain and C-
terminal domain (including the half domain))

The combinations of the first fusion protein and the second fusion protein used in the base editors shown in FIG. 2 are as follows.

| Base Editor ID | First Fusion Protein | Second Fusion Protein |
|---|---|---|
| 17N + 56C | SEQ ID NO: 78 | SEQ ID NO: 90 |
| 17N + 57C | SEQ ID NO: 78 | SEQ ID NO: 91 |
| 18N + 244Cv | SEQ ID NO: 79 | SEQ ID NO: 92 |
| 18N + 246Cv | SEQ ID NO: 79 | SEQ ID NO: 93 |
| 18N + 247Cv | SEQ ID NO: 79 | SEQ ID NO: 94 |
| 18N + 56C | SEQ ID NO: 79 | SEQ ID NO: 90 |
| 18N + 57C | SEQ ID NO: 79 | SEQ ID NO: 91 |
| 229Nv + 43C | SEQ ID NO: 80 | SEQ ID NO: 95 |
| 229Nv + 56C | SEQ ID NO: 80 | SEQ ID NO: 90 |
| 229Nv + 57C | SEQ ID NO: 80 | SEQ ID NO: 91 |
| 231Nv + 48C | SEQ ID NO: 81 | SEQ ID NO: 96 |
| 231Nv + 57C | SEQ ID NO: 81 | SEQ ID NO: 91 |
| 232Nv + 53C | SEQ ID NO: 82 | SEQ ID NO: 97 |
| 232Nv + 56C | SEQ ID NO: 82 | SEQ ID NO: 90 |
| 17C + 248Nv | SEQ ID NO: 83 | SEQ ID NO: 88 |
| 17C + 57N | SEQ ID NO: 83 | SEQ ID NO: 87 |
| 17C + 249Nv | SEQ ID NO: 83 | SEQ ID NO: 89 |
| 18C + 248Nv | SEQ ID NO: 84 | SEQ ID NO: 88 |
| 18C + 56N | SEQ ID NO: 84 | SEQ ID NO: 86 |
| 18C + 57N | SEQ ID NO: 84 | SEQ ID NO: 87 |
| 229Cv + 248Nv | SEQ ID NO: 85 | SEQ ID NO: 88 |

### Example 7: Construction of TALED and ZFD for Correction of m.G11778A Mutation

TALED and ZFD (ZF deaminase) were constructed so that the editing window containing the G11778A point mutation in mitochondrial DNA ND4 to be corrected would span 1 to 20 bp. The method for constructing TALED is as described in Example 4. To construct ZFD, the sequence encoding the zinc finger proteins that bind to the target site was codon-optimized for expression in humans, and the double-stranded DNA sequence was synthesized as a gBlock DNA fragment from IDT (Integrated DNA Technologies). Using the synthesized gBlock DNA fragment and the expression vector backbone (containing MTS, HA tag, NES, DddAₜₒₓ 1397N or 1397C, and TadA8e) as templates, the DNA fragments required for Gibson assembly were amplified using PrimeSTAR® GXL DNA polymerase (TAKARA) and purified using a PCR SV mini kit (GeneAll). The purified DNA fragments were assembled using the HiFi DNA Assembly Kit (NEB), and the transformation into competent DH5a (enzynomics) E. coli cells and confirmation of the base sequence by Sanger sequencing (Macrogen) were performed as described in Example 4.

The first fusion protein and the second fusion protein were linked in the following order: [MTS]-[tag]-[TALE protein]-[linker]-[DddAₜₒₓ split], [MTS]-[tag]-[TALE protein]-[linker]-[DddAₜₒₓ split]-[linker]-[TadA8e], [MTS]-[tag]-[NES]-[linker]-[DddAₜₒₓ split]-[linker]-[ZF protein], or [MTS]-[tag]-[NES]-[linker]-[DddAₜₒₓ split]-[linker]-[TadA8e]-[linker]-[ZF protein]. As tags, 3X HA (SEQ ID NO: 136) or 3X FLAG (SEQ ID NO: 137) was used for fusion proteins containing TALE proteins, and 1X HA (SEQ ID NO: 138) was used for fusion proteins containing ZF proteins. The proteins used are located between the CMV promoter and the T7 promoter and terminator sequence.

### Example 8: Transfection of Urine-Derived Cells from a Patient Carrying the G11778A Mutation in the Mitochondrial ND4 Gene with a Fusion Protein

Cells were isolated from the urine of an LHON patient carrying the G11778A point mutation in the mitochondrial ND4 gene to obtain primary cells, which were aliquoted at passage 2 and cryopreserve.

The method for culturing urine-derived cells (UDC) from an LHON patient with the G11778A point mutation is as described in Example 5. Plasmids encoding the first fusion protein and the second fusion protein, 1 ug each for a total of 2 ug, were transfected into UDC cells (1.0×10⁴ cells) using the NEON 10 uL TRANSFECTION KIT (Invitrogen) and the NEON Transfection System (Invitrogen) by electroporation (1350V, 30 ms, 1pulse). The transfected UDC cells were placed into 8-well Clear TC-Treated Multiple Well Plates (Corning) pre-coated with 0.1% gelatin and pre-filled with culture medium. Transfected cells were maintained at 37° C. in 5% CO₂, with replacement of the culture medium. The cells were harvested after 6 days, the culture medium was removed, and the cells were lysed, and the process is the same as described in Example 5.

### Example 9: Sequencing and Base Editing Efficiency Measurement to Confirm Correction of m.g11778a Mutation

The reaction product obtained in Example 8 was used as a template without purification, and the sequence was analyzed using a targeted deep sequencing technique to analyze the base editing ratio of the target site. The method from library preparation to deep sequencing was as described in Example 6.

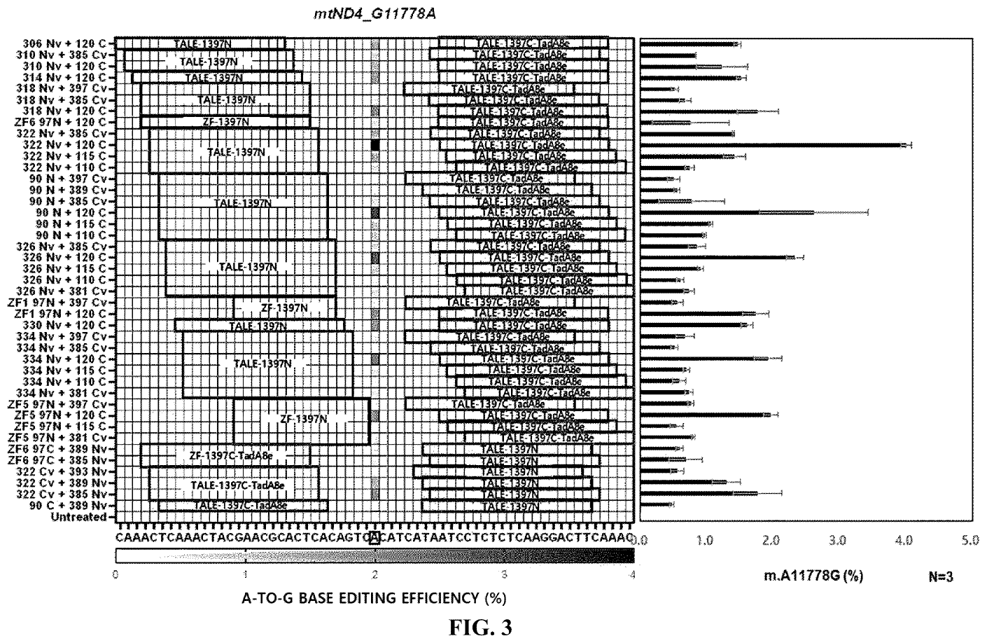

To screen the efficiency of the developed base editors in correcting m.A11778G, combinations of TALED and TALED, hybrid combinations of TALED and ZFD, and combinations of ZFD and ZFD were transfected. The DNA binding sites of 42 base editor combinations with high base editing efficiency among the above combinations and the efficiencies of m.A11778G correction were shown in FIG. 3.

The amino acid sequences of the first fusion protein and the second fusion protein used in the combinations of 42 base editors confirmed to have high base editing efficiency are as follows.

First Fusion Protein
SEQ ID NO: 100
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPDQVVAIASNIGGKQALETVQRLLPVLCQAHGLTPAQVV
AIASNIGGKQALETVQRLLPVLCQDHGLTPAQVVAIASNIGGKQALETVQRLLPVLCQAH
GLTPAQVVAIASHDGGKQALETVQRLLPVLCQDHGLTPAQVVAIASNGGGKQALETVQRL
LPVLCQDHGLTPAQVVAIASHDGGKQALETVQRLLPVLCQAHGLTPDQVVAIASNIGGKQ
ALETVQRLLPVLCQAHGLTPAQVVAIASNIGGKQALETVQRLLPVLCQDHGLTPAQVVAI
ASNIGGKQALETVQRLLPVLCQAHGLTPDQVVAIASHDGGKQALETVQRLLPVLCQAHGL
TPDQVVAIASNGGGKQALETVQRLLPVLCQDHGLTPAQVVAIASNIGGKQALETVQRLLP
VLCQDHGLTPEQVVAIASHDGGKQALETVQRLLPVLCQAHGLTPAQVVAIASNNGGKQAL
ETVQRLLPVLCQAHGLTPAQVVAIASNIGGKQALETVQRLLPVLCQDHGLTPDQVVAIAS
NIGGKQALETVQRLLPVLCQDHGLTPDQVVAIASHDGGKQALETVQRLLPVLCQAHGLTP
AQVVAIASNNGGKQALETVQRLLPVLCQDHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 101
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPDQVVAIASNIGGKQALETVQRLLPVLCQAHGLTPDQVV
AIASNIGGKQALETVQRLLPVLCQAHGLTPAQVVAIASHDGGKQALETVQRLLPVLCQAH
GLTPAQVVAIASNGGGKQALETVQRLLPVLCQDHGLTPEQVVAIASHDGGKQALETVQRL
LPVLCQAHGLTPAQVVAIASNIGGKQALETVQRLLPVLCQDHGLTPDQVVAIASNIGGKQ
ALETVQRLLPVLCQAHGLTPDQVVAIASNIGGKQALETVQRLLPVLCQAHGLTPAQVVAI
ASHDGGKQALETVQRLLPVLCQAHGLTPEQVVAIASNGGGKQALETVQRLLPVLCQAHGL
TPDQVVAIASNIGGKQALETVQRLLPVLCQAHGLTPAQVVAIASHDGGKQALETVQRLLP
VLCQDHGLTPEQVVAIASNNGGKQALETVQRLLPVLCQAHGLTPDQVVAIASNIGGKQAL
ETVQRLLPVLCQDHGLTPDQVVAIASNIGGKQALETVQRLLPVLCQAHGLTPAQVVAIAS
HDGGKQALETVQRLLPVLCQAHGLTPDQVVAIASNNGGKQALETVQRLLPVLCQDHGLTP
DQVVAIASHDGGKQALETVQRLLPVLCQDHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 102
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPEQVVAIASNIGGKQALETVQRLLPVLCQAHGLTPDQVV
AIASHDGGKQALETVQRLLPVLCQAHGLTPDQVVAIASNGGGKQALETVQRLLPVLCQDH
GLTPAQVVAIASHDGGKQALETVQRLLPVLCQDHGLTPEQVVAIASNIGGKQALETVQRL
LPVLCQAHGLTPAQVVAIASNIGGKQALETVQRLLPVLCQDHGLTPEQVVAIASNIGGKQ
ALETVQRLLPVLCQAHGLTPDQVVAIASHDGGKQALETVQRLLPVLCQAHGLTPDQVVAI
ASNGGGKQALETVQRLLPVLCQAHGLTPDQVVAIASNIGGKQALETVQRLLPVLCQDHGL
TPDQVVAIASHDGGKQALETVQRLLPVLCQAHGLTPAQVVAIASNNGGKQALETVQRLLP
VLCQDHGLTPDQVVAIASNIGGKQALETVQRLLPVLCQAHGLTPDQVVAIASNIGGKQAL
ETVQRLLPVLCQAHGLTPAQVVAIASHDGGKQALETVQRLLPVLCQAHGLTPAQVVAIAS
NNGGKQALETVQRLLPVLCQAHGLTPDQVVAIASHDGGKQALETVQRLLPVLCQDHGLTP
AQVVAIASNIGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 103
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPAQVVAIASHDGGKQALETVQRLLPVLCQDHGLTPAQVV
AIASNGGGKQALETVQRLLPVLCQDHGLTPAQVVAIASHDGGKQALETVQRLLPVLCQAH
GLTPDQVVAIASNIGGKQALETVQRLLPVLCQAHGLTPAQVVAIASNIGGKQALETVQRL
LPVLCQDHGLTPAQVVAIASNIGGKQALETVQRLLPVLCQAHGLTPDQVVAIASHDGGKQ
ALETVQRLLPVLCQAHGLTPDQVVAIASNGGGKQALETVQRLLPVLCQDHGLTPAQVVAI
ASNIGGKQALETVQRLLPVLCQAHGLTPEQVVAIASHDGGKQALETVQRLLPVLCQAHGL
TPAQVVAIASNNGGKQALETVQRLLPVLCQAHGLTPAQVVAIASNIGGKQALETVQRLLP
VLCQDHGLTPDQVVAIASNIGGKQALETVQRLLPVLCQDHGLTPDQVVAIASHDGGKQAL
ETVQRLLPVLCQAHGLTPAQVVAIASNNGGKQALETVQRLLPVLCQDHGLTPDQVVAIAS
HDGGKQALETVQRLLPVLCQAHGLTPDQVVAIASNIGGKQALETVQRLLPVLCQAHGLTP
AQVVAIASHDGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein
including the N-terminal domain and C-terminal domain.
(including the half domain))
SEQ ID NO: 104
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPAQVVAIASNGGGKQALETVQRLLPVLCQDHGLTPEQVV
AIASHDGGKQALETVQRLLPVLCQAHGLTPAQVVAIASNIGGKQALETVQRLLPVLCQDH
GLTPDQVVAIASNIGGKQALETVQRLLPVLCQAHGLTPDQVVAIASNIGGKQALETVQRL
LPVLCQAHGLTPAQVVAIASHDGGKQALETVQRLLPVLCQAHGLTPEQVVAIASNGGGKQ
ALETVQRLLPVLCQAHGLTPDQVVAIASNIGGKQALETVQRLLPVLCQAHGLTPAQVVAI
ASHDGGKQALETVQRLLPVLCQAHGLTPEQVVAIASNNGGKQALETVQRLLPVLCQAHGL
TPDQVVAIASNIGGKQALETVQRLLPVLCQDHGLTPDQVVAIASNIGGKQALETVQRLLP
VLCQAHGLTPAQVVAIASHDGGKQALETVQRLLPVLCQAHGLTPDQVVAIASNNGGKQAL
ETVQRLLPVLCQDHGLTPDQVVAIASHDGGKQALETVQRLLPVLCQDHGLTPEQVVAIAS
NIGGKQALETVQRLLPVLCQAHGLTPDQVVAIASHDGGKQALETVQRLLPVLCQAHGLTP
DQVVAIASNGGGKQALETVQRLLPVLCQDHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 105
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKQWSGARALEALLTVAGELRGPPLQLDTGQLLKIAKRGG
VTAVEAVHAWRNALTGAPLNLTPAQVVAIASHDGGKQALETVQRLLPVLCQDHGLTPEQV
VAIASNIGGKQALETVQRLLPVLCQAHGLTPAQVVAIASNIGGKQALETVQRLLPVLCQD
HGLTPEQVVAIASNIGGKQALETVQRLLPVLCQAHGLTPDQVVAIASHDGGKQALETVQR
LLPVLCQAHGLTPDQVVAIASNGGGKQALETVQRLLPVLCQDHGLTPDQVVAIASNIGGK
QALETVQRLLPVLCQDHGLTPDQVVAIASHDGGKQALETVQRLLPVLCQAHGLTPAQVVA
IASNNGGKQALETVQRLLPVLCQAHGLTPDQVVAIASNIGGKQALETVQRLLPVLCQAHG
LTPDQVVAIASNIGGKQALETVQRLLPVLCQAHGLTPAQVVAIASHDGGKQALETVQRLL
PVLCQAHGLTPAQVVAIASNNGGKQALETVQRLLPVLCQAHGLTPDQVVAIASHDGGKQA
LETVQRLLPVLCQDHGLTPAQVVAIASNIGGKQALETVQRLLPVLCQAHGLTPAQVVAIA
SHDGGKQALETVQRLLPVLCQDHGLTPAQVVAIASNGGGKQALETVQRLLPVLCQDHGLT
PAQVVAIASHDGGKQALETVQRLLPVLCQAHGLTPEQVVAIASNGGGKQALESIVAQLSR
PDPALAALTNDHLVALACLGGRPALDAVKKGLGGSGSGSYALGPYQISAPQLPAYNGQTV
GTFYYVNDAGGLESKVFSSGGPTPYPNYANAGHVEGQSALFMRDNGISEGLVFHNNPEGT
CGFCVNMTETLLPENAKMTVVPPEG.
(The underlined portion corresponds to the TALE
protein including the N-terminal domain and C-terminal domain.
(including the half domain))
SEQ ID NO: 106
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPDQVVAIASNIGGKQALETVQRLLPVLCQAHGLTPAQVV
AIASNIGGKQALETVQRLLPVLCQDHGLTPAQVVAIASNIGGKQALETVQRLLPVLCQAH
GLTPDQVVAIASHDGGKQALETVQRLLPVLCQAHGLTPDQVVAIASNGGGKQALETVQRL
LPVLCQDHGLTPAQVVAIASNIGGKQALETVQRLLPVLCQDHGLTPEQVVAIASHDGGKQ
ALETVQRLLPVLCQAHGLTPAQVVAIASNNGGKQALETVQRLLPVLCQAHGLTPAQVVAI
ASNIGGKQALETVQRLLPVLCQAHGLTPDQVVAIASNIGGKQALETVQRLLPVLCQDHGL
TPDQVVAIASHDGGKQALETVQRLLPVLCQAHGLTPAQVVAIASNNGGKQALETVQRLLP
VLCQDHGLTPDQVVAIASHDGGKQALETVQRLLPVLCQAHGLTPDQVVAIASNIGGKQAL
ETVQRLLPVLCQAHGLTPAQVVAIASHDGGKQALETVQRLLPVLCQAHGLTPAQVVAIAS
NGGGKQALETVQRLLPVLCQDHGLTPEQVVAIASHDGGKQALETVQRLLPVLCQAHGLTP
AQVVAIASNIGGKQALETVQRLLPVLCQDHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 107
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPDQVVAIASNIGGKQALETVQRLLPVLCQAHGLTPDQVV
AIASNIGGKQALETVQRLLPVLCQAHGLTPAQVVAIASHDGGKQALETVQRLLPVLCQAH
GLTPEQVVAIASNGGGKQALETVQRLLPVLCQAHGLTPDQVVAIASNIGGKQALETVQRL
LPVLCQAHGLTPAQVVAIASHDGGKQALETVQRLLPVLCQDHGLTPEQVVAIASNNGGKQ
ALETVQRLLPVLCQAHGLTPDQVVAIASNIGGKQALETVQRLLPVLCQDHGLTPDQVVAI
ASNIGGKQALETVQRLLPVLCQAHGLTPAQVVAIASHDGGKQALETVQRLLPVLCQAHGL
TPDQVVAIASNNGGKQALETVQRLLPVLCQDHGLTPDQVVAIASHDGGKQALETVQRLLP
VLCQDHGLTPEQVVAIASNIGGKQALETVQRLLPVLCQAHGLTPDQVVAIASHDGGKQAL
ETVQRLLPVLCQAHGLTPDQVVAIASNGGGKQALETVQRLLPVLCQDHGLTPDQVVAIAS
HDGGKQALETVQRLLPVLCQAHGLTPDQVVAIASNIGGKQALETVQRLLPVLCQAHGLTP
AQVVAIASHDGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 108
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPEQVVAIASNIGGKQALETVQRLLPVLCQAHGLTPDQVV
AIASHDGGKQALETVQRLLPVLCQAHGLTPDQVVAIASNGGGKQALETVQRLLPVLCQDH
GLTPDQVVAIASNIGGKQALETVQRLLPVLCQDHGLTPDQVVAIASHDGGKQALETVQRL
LPVLCQAHGLTPAQVVAIASNNGGKQALETVQRLLPVLCQDHGLTPDQVVAIASNIGGKQ
ALETVQRLLPVLCQAHGLTPDQVVAIASNIGGKQALETVQRLLPVLCQAHGLTPAQVVAI
ASHDGGKQALETVQRLLPVLCQAHGLTPAQVVAIASNNGGKQALETVQRLLPVLCQAHGL
TPDQVVAIASHDGGKQALETVQRLLPVLCQDHGLTPAQVVAIASNIGGKQALETVQRLLP
VLCQAHGLTPAQVVAIASHDGGKQALETVQRLLPVLCQDHGLTPAQVVAIASNGGGKQAL
ETVQRLLPVLCQDHGLTPAQVVAIASHDGGKQALETVQRLLPVLCQAHGLTPDQVVAIAS
NIGGKQALETVQRLLPVLCQDHGLTPDQVVAIASHDGGKQALETVQRLLPVLCQDHGLTP
AQVVAIASNIGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 109
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNGGGKQALETVQRLLPVLCQDHGLTPEQVVAIASHDGG
KQALETVQRLLPVLCQAHGLTPAQVVAIASNIGGKQALETVQRLLPVLCQDHGLTPDQVV
AIASNIGGKQALETVQRLLPVLCQAHGLTPDQVVAIASNIGGKQALETVQRLLPVLCQAH
GLTPAQVVAIASHDGGKQALETVQRLLPVLCQAHGLTPEQVVAIASNGGGKQALETVQRL
LPVLCQAHGLTPDQVVAIASNIGGKQALETVQRLLPVLCQAHGLTPAQVVAIASHDGGKQ
ALETVQRLLPVLCQAHGLTPEQVVAIASNNGGKQALETVQRLLPVLCQAHGLTPDQVVAI
ASNIGGKQALETVQRLLPVLCQDHGLTPDQVVAIASNIGGKQALETVQRLLPVLCQAHGL
TPAQVVAIASHDGGKQALETVQRLLPVLCQAHGLTPDQVVAIASNNGGKQALETVQRLLP
VLCQDHGLTPDQVVAIASHDGGKQALETVQRLLPVLCQDHGLTPEQVVAIASNIGGKQAL
ETVQRLLPVLCQAHGLTPDQVVAIASHDGGKQALETVQRLLPVLCQAHGLTPDQVVAIAS
NGGGKQALETVQRLLPVLCQDHGLTPEQVVAIASNGGGKQALESIVAQLSRPDPALAALT
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))
SEQ ID NO: 110
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASHDGGKQALETVQRLLPVLCQDHGLTPEQVVAIASNIG
GKQALETVQRLLPVLCQAHGLTPAQVVAIASNIGGKQALETVQRLLPVLCQDHGLTPEQV
VAIASNIGGKQALETVQRLLPVLCQAHGLTPDQVVAIASHDGGKQALETVQRLLPVLCQA
HGLTPDQVVAIASNGGGKQALETVQRLLPVLCQDHGLTPDQVVAIASNIGGKQALETVQR
LLPVLCQDHGLTPDQVVAIASHDGGKQALETVQRLLPVLCQAHGLTPAQVVAIASNNGGK
QALETVQRLLPVLCQAHGLTPDQVVAIASNIGGKQALETVQRLLPVLCQAHGLTPDQVVA
IASNIGGKQALETVQRLLPVLCQAHGLTPAQVVAIASHDGGKQALETVQRLLPVLCQAHG
LTPAQVVAIASNNGGKQALETVQRLLPVLCQAHGLTPDQVVAIASHDGGKQALETVQRLL
PVLCQDHGLTPAQVVALASNIGGKQALETVQRLLPVLCQAHGLTPAQVVAIASHDGGKQA
LETVQRLLPVLCQDHGLTPAQVVAIASNGGGKQALETVQRLLPVLCQDHGLTPAQVVAIA
SHDGGKQALETVQRLLPVLCQAHGLTPEQVVAIASNGGGKQALESIVAQLSRPDPALAAL
INDHLVALACLGGRPALDAVKKGLLVGSAIPVKRGATGETKVFTGNSNSPKSPTKGGCSG
SETPGTSESATPESSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVFGVR
NSKRGAAGSLMNVLNYPGMNHRVEITEGILADECAALLCDFYRMPRQVFNAQKKAQSSIN.
(The underlined portion corresponds to the TALE
protein including the N-terminal
domain and C-terminal domain.
(including the half domain))
SEQ ID NO: 111
MLGFVGRVAAAPASGALRRLTPSASLPPAQLLLRAAPTAVHPVRDYAAQYPYDVPDYAVD
EMTKKFGTLTIHDTEKAAEFGIHGVPAAMGGSYALGPYQISAPQLPAYNGQTVGTFYYVN
DAGGLESKVFSSGGPTPYPNYANAGHVEGQSALFMRDNGISEGLVFHNNPEGTCGFCVNM
TETLLPENAKMTVVPPEGSGTPHEVGVYTLSGTPHEVGVYTLYKCPECGKSFSSKKALTE
HQRTHTGEKPYKCPECGKSFSTHLDLIRHQRTHTGEKPYKCPECGKSFSHTGHLLEHQRT
HTGEKPFECKDCGKAFIQKSNLIRHQRTH.
(The underlined portion corresponds to the zinc finger protein)
SEQ ID NO: 112
MLGFVGRVAAAPASGALRRLTPSASLPPAQLLLRAAPTAVHPVRDYAAQYPYDVPDYAVD
EMTKKFGTLTIHDTEKAAEFGIHGVPAAMGGSYALGPYQISAPQLPAYNGQTVGTFYYVN
DAGGLESKVFSSGGPTPYPNYANAGHVEGQSALFMRDNGISEGLVFHNNPEGTCGFCVNM
TETLLPENAKMTVVPPEGSGTPHEVGVYTLSGTPHEVGVYTLYSCGICGKSFSDSSAKRR
HCILHTGEKPYKCPECGKSFSSPADLTRHQRTHLRQKDGERPYKCPECGKSFSTHLDLIR
HQRTHTGEKPYKCPECGKSFSHTGHLLEHQRTHTGEKPFECKDCGKAFIQKSNLIRHQRT
H.
(The underlined portion corresponds to the zinc
finger protein)
SEQ ID NO: 113
MLGFVGRVAAAPASGALRRLTPSASLPPAQLLLRAAPTAVHPVRDYAAQYPYDVPDYAVD
EMTKKFGTLTIHDTEKAAEFGIHGVPAAMGGSYALGPYQISAPQLPAYNGQTVGTFYYVN
DAGGLESKVFSSGGPTPYPNYANAGHVEGQSALFMRDNGISEGLVFHNNPEGTCGFCVNM
TETLLPENAKMTVVPPEGSGTPHEVGVYTLSGTPHEVGVYTLYKCPECGKSFSTHLDLIR
HQRTHTGEKPYKCPECGKSFSHTGHLLEHQRTHTGEKPFECKDCGKAFIQKSNLIRHQRT
HLRQKDGGGSERPYKCPECGKSFSTHLDLIRHQRTHTGEKPYKCDECGKNFTQSSNLIVH
KRIHTGEKPYKCPECGKSFSTHLDLIRHQRTH.
(The underlined portion corresponds to the zinc finger protein)
SEQ ID NO: 114
MLGFVGRVAAAPASGALRRLTPSASLPPAQLLLRAAPTAVHPVRDYAAQYPYDVPDYAVD
EMTKKFGTLTIHDTEKAAEFGIHGVPAAMGGSAIPVKRGATGETKVFTGNSNSPKSPTKG
GCSGSETPGTSESATPESSEVEFSHEYWMRHALTLAKRARDEREVPVGAVLVLNNRVIGE
GWNRAIGLHDPTAHAEIMALRQGGLVMQNYRLIDATLYVTFEPCVMCAGAMIHSRIGRVV
FGVRNSKRGAAGSLMNVLNYPGMNHRVEITEGILADECAALLCDFYRMPRQVFNAQKKAQ
SSINSGTPHEVGVYTLSGTPHEVGVYTLYKCPECGKSFSTHLDLIRHQRTHTGEKPYKCP
ECGKSFSHTGHLLEHQRTHTGEKPFECKDCGKAFIQKSNLIRHQRTHLRQKDGGGSERPY
KCPECGKSFSTHLDLIRHQRTHTGEKPYKCDECGKNFTQSSNLIVHKRIHTGEKPYKCPE
CGKSFSTHLDLIRHQRTH.
(The underlined portion corresponds to the zinc finger protein)
Second Fusion Protein
SEQ ID NO: 115
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPAQVVAIASNIGGKQALETVQRLLPVLCQAHGLTPEQVV
AIASNNGGKQALETVQRLLPVLCQAHGLTPDQVVAIASNGGGKQALETVQRLLPVLCQDH
GLTPDQVVAIASHDGGKQALETVQRLLPVLCQAHGLTPAQVVAIASHDGGKQALETVQRL
LPVLCQDHGLTPAQVVAIASNGGGKQALETVQRLLPVLCQDHGLTPAQVVAIASNGGGKQ
ALETVQRLLPVLCQAHGLTPAQVVAIASNNGGKQALETVQRLLPVLCQDHGLTPAQVVAI
ASNIGGKQALETVQRLLPVLCQAHGLTPDQVVAIASNNGGKQALETVQRLLPVLCQDHGL
TPAQVVAIASNIGGKQALETVQRLLPVLCQAHGLTPEQVVAIASNNGGKQALETVQRLLP
VLCQAHGLTPAQVVAIASNIGGKQALETVQRLLPVLCQAHGLTPAQVVAIASNNGGKQAL
ETVQRLLPVLCQDHGLTPAQVVAIASNNGGKQALETVQRLLPVLCQAHGLTPAQVVAIAS
NIGGKQALETVQRLLPVLCQAHGLTPDQVVAIASNGGGKQALETVQRLLPVLCQAHGLTP
DQVVAIASNGGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 116
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPAQVVAIASNIGGKQALETVQRLLPVLCQDHGLTPDQVV
AIASNIGGKQALETVQRLLPVLCQDHGLTPDQVVAIASNNGGKQALETVQRLLPVLCQAH
GLTPAQVVAIASNGGGKQALETVQRLLPVLCQDHGLTPAQVVAIASHDGGKQALETVQRL
LPVLCQAHGLTPEQVVAIASHDGGKQALETVQRLLPVLCQAHGLTPEQVVAIASNGGGKQ
ALETVQRLLPVLCQAHGLTPAQVVAIASNGGGKQALETVQRLLPVLCQAHGLTPDQVVAI
ASNNGGKQALETVQRLLPVLCQAHGLTPDQVVAIASNIGGKQALETVQRLLPVLCQDHGL
TPAQVVAIASNNGGKQALETVQRLLPVLCQDHGLTPAQVVAIASNIGGKQALETVQRLLP
VLCQAHGLTPDQVVAIASNNGGKQALETVQRLLPVLCQDHGLTPAQVVAIASNIGGKQAL
ETVQRLLPVLCQAHGLTPEQVVAIASNNGGKQALETVQRLLPVLCQAHGLTPAQVVAIAS
NNGGKQALETVQRLLPVLCQDHGLTPAQVVAIASNIGGKQALETVQRLLPVLCQAHGLTP
EQVVAIASNGGGKQALETVQRLLPVLCQAHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG
(The underlined portion corresponds to the TALE
protein including the N-terminal domain and C-terminal domain.
(including the half domain))
SEQ ID NO: 117
MALSRAVCGTSRQLAPVLGYLGSRQKHSLPDYPYDVPDYAGYPYDVPDYAGYPYDVPDYA
GIRIQDLRTLGYSQQQQEKIKPKVRSTVAQHHEALVGHGFTHAHIVALSQHPAALGTVAV
KYQDMIAALPEATHEAIVGVGKYHGARALEALLTVAGELRGPPLQLDTGQLLKIAKRGGV
TAVEAVHAWRNALTGAPLNLTPAQVVAIASNNGGKQALETVQRLLPVLCQDHGLTPAQVV
AIASNGGGKQALETVQRLLPVLCQAHGLTPDQVVAIASHDGGKQALETVQRLLPVLCQDH
GLTPAQVVAIASHDGGKQALETVQRLLPVLCQAHGLTPDQVVAIASNGGGKQALETVQRL
LPVLCQAHGLTPAQVVAIASNGGGKQALETVQRLLPVLCQDHGLTPDQVVAIASNNGGKQ
ALETVQRLLPVLCQDHGLTPAQVVAIASNIGGKQALETVQRLLPVLCQAHGLTPEQVVAI
ASNNGGKQALETVQRLLPVLCQAHGLTPDQVVAIASNIGGKQALETVQRLLPVLCQDHGL
TPAQVVAIASNNGGKQALETVQRLLPVLCQDHGLTPAQVVAIASNIGGKQALETVQRLLP
VLCQAHGLTPAQVVAIASNNGGKQALETVQRLLPVLCQAHGLTPDQVVAIASNNGGKQAL
ETVQRLLPVLCQDHGLTPAQVVAIASNIGGKQALETVQRLLPVLCQAHGLTPEQVVAIAS
NGGGKQALETVQRLLPVLCQAHGLTPDQVVAIASNGGGKQALETVQRLLPVLCQDHGLTP
DQVVAIASNIGGKQALETVQRLLPVLCQDHGLTPEQVVAIASNGGGKQALESIVAQLSRP
DPALAALTNDHLVALACLGGRPALDAVKKGLGGSGSGSYALGPYQISAPQLPAYNGQTVG
TFYYVNDAGGLESKVFSSGGPTPYPNYANAGHVEGQSALFMRDNGISEGLVFHNNPEGTC
GFCVNMTETLLPENAKMTVVPPEG.
(The underlined portion corresponds to the TALE
protein including the N-terminal domain and C-terminal domain.
(including the half domain))
SEQ ID NO: 118
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPEQVVAIASNNGGKQALETVQRLLPVLCQAHGLTPDQVVAIASNIG
GKQALETVQRLLPVLCQDHGLTPDQVVAIASNIGGKQALETVQRLLPVLCQAHGLTPAQV
VAIASNNGGKQALETVQRLLPVLCQDHGLTPAQVVAIASNGGGKQALETVQRLLPVLCQA
HGLTPDQVVAIASHDGGKQALETVQRLLPVLCQDHGLTPAQVVAIASHDGGKQALETVQR
LLPVLCQAHGLTPDQVVAIASNGGGKQALETVQRLLPVLCQAHGLTPAQVVAIASNGGGK
QALETVQRLLPVLCQAHGLTPDQVVAIASNNGGKQALETVQRLLPVLCQDHGLTPAQVVA
IASNIGGKQALETVQRLLPVLCQAHGLTPEQVVAIASNNGGKQALETVQRLLPVLCQAHG
LTPDQVVAIASNIGGKQALETVQRLLPVLCQDHGLTPAQVVAIASNNGGKQALETVQRLL
PVLCQDHGLTPAQVVAIASNIGGKQALETVQRLLPVLCQAHGLTPAQVVAIASNNGGKQA
LETVQRLLPVLCQAHGLTPDQVVAIASNNGGKQALETVQRLLPVLCQDHGLTPAQVVAIA
SNIGGKQALETVQRLLPVLCQAHGLTPEQVVAIASNGGGKQALESIVAQLSRPDPALAAL
INDHLVALACLGGRPALDAVKKGLLVGSAIPVKRGATGETKVFTGNSNSPKSPTKGGCSG
SETPGTSESATPESSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVFGVR
NSKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))
SEQ ID NO: 119
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNIGGKQALETVQRLLPVLCQDHGLTPDQVVAIASNIGG
KQALETVQRLLPVLCQDHGLTPDQVVAIASNNGGKQALETVQRLLPVLCQAHGLTPAQVV
AIASNGGGKQALETVQRLLPVLCQDHGLTPAQVVAIASHDGGKQALETVQRLLPVLCQAH
GLTPEQVVAIASHDGGKQALETVQRLLPVLCQAHGLTPEQVVAIASNGGGKQALETVQRL
LPVLCQAHGLTPAQVVAIASNGGGKQALETVQRLLPVLCQAHGLTPDQVVAIASNNGGKQ
ALETVQRLLPVLCQAHGLTPDQVVAIASNIGGKQALETVQRLLPVLCQDHGLTPAQVVAI
ASNNGGKQALETVQRLLPVLCQDHGLTPAQVVAIASNIGGKQALETVQRLLPVLCQAHGL
TPDQVVAIASNNGGKQALETVQRLLPVLCQDHGLTPAQVVAIASNIGGKQALETVQRLLP
VLCQAHGLTPEQVVAIASNNGGKQALETVQRLLPVLCQAHGLTPAQVVAIASNNGGKQAL
ETVQRLLPVLCQDHGLTPAQVVAIASNIGGKQALETVQRLLPVLCQAHGLTPEQVVAIAS
NGGGKQALETVQRLLPVLCQAHGLTPEQVVAIASNGGGKQALESIVAQLSRPDPALAALT
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))
SEQ ID NO: 120
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNGGGKQALETVQRLLPVLCQDHGLTPAQVVAIASHDGG
KQALETVQRLLPVLCQAHGLTPEQVVAIASHDGGKQALETVQRLLPVLCQAHGLTPEQVV
AIASNGGGKQALETVQRLLPVLCQAHGLTPAQVVAIASNGGGKQALETVQRLLPVLCQAH
GLTPDQVVAIASNNGGKQALETVQRLLPVLCQDHGLTPDQVVAIASNIGGKQALETVQRL
LPVLCQDHGLTPAQVVAIASNNGGKQALETVQRLLPVLCQDHGLTPAQVVAIASNIGGKQ
ALETVQRLLPVLCQAHGLTPDQVVAIASNNGGKQALETVQRLLPVLCQDHGLTPAQVVAI
ASNIGGKQALETVQRLLPVLCQAHGLTPEQVVAIASNNGGKQALETVQRLLPVLCQAHGL
TPAQVVAIASNNGGKQALETVQRLLPVLCQDHGLTPAQVVAIASNIGGKQALETVQRLLP
VLCQAHGLTPEQVVAIASNGGGKQALETVQRLLPVLCQAHGLTPDQVVAIASNGGGKQAL
ETVQRLLPVLCQAHGLTPDQVVAIASNIGGKQALETVQRLLPVLCQDHGLTPDQVVAIAS
NGGGKQALETVQRLLPVLCQAHGLTPEQVVAIASNGGGKQALESIVAQLSRPDPALAALT
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein
including the N-terminal
domain and C-terminal domain.
(including the half domain))
SEQ ID NO: 121
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPAQVVAIASNGGGKQALETVQRLLPVLCQAHGLTPAQVVAIASNNG
GKQALETVQRLLPVLCQDHGLTPAQVVAIASNIGGKQALETVQRLLPVLCQDHGLTPAQV
VAIASNIGGKQALETVQRLLPVLCQAHGLTPEQVVAIASNNGGKQALETVQRLLPVLCQA
HGLTPDQVVAIASNGGGKQALETVQRLLPVLCQDHGLTPDQVVAIASHDGGKQALETVQR
LLPVLCQAHGLTPAQVVAIASHDGGKQALETVQRLLPVLCQDHGLTPAQVVAIASNGGGK
QALETVQRLLPVLCQAHGLTPAQVVAIASNGGGKQALETVQRLLPVLCQAHGLTPAQVVA
IASNNGGKQALETVQRLLPVLCQDHGLTPAQVVAIASNIGGKQALETVQRLLPVLCQDHG
LTPDQVVAIASNNGGKQALETVQRLLPVLCQDHGLTPAQVVAIASNIGGKQALETVQRLL
PVLCQAHGLTPEQVVAIASNNGGKQALETVQRLLPVLCQAHGLTPAQVVAIASNIGGKQA
LETVQRLLPVLCQAHGLTPAQVVAIASNNGGKQALETVQRLLPVLCQDHGLTPAQVVAIA
SNNGGKQALETVQRLLPVLCQAHGLTPEQVVAIASNGGGKQALESIVAQLSRPDPALAAL
TNDHLVALACLGGRPALDAVKKGLLVGSAIPVKRGATGETKVFTGNSNSPKSPTKGGCSG
SETPGTSESATPESSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVFGVR
NSKRGAAGSLMNVLNYPGMNHRVEITEGILADECAALLCDFYRMPRQVFNAQKKAQSSIN
(The underlined portion corresponds to the TALE
protein including the N-terminal domain
and C-terminal domain.
(including the half domain))
SEQ ID NO: 122
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKQWSGARALEALLTVAGELRGPPLQLDTGQLLKIAKRGGVTAVEAVH
AWRNALTGAPLNLTPEQVVAIASNGGGKQALETVQRLLPVLCQAHGLTPAQVVAIASNGG
GKQALETVQRLLPVLCQAHGLTPDQVVAIASNNGGKQALETVQRLLPVLCQDHGLTPAQV
VAIASNIGGKQALETVQRLLPVLCQDHGLTPDQVVAIASNIGGKQALETVQRLLPVLCQD
HGLTPDQVVAIASNNGGKQALETVQRLLPVLCQAHGLTPAQVVAIASNGGGKQALETVQR
LLPVLCQDHGLTPAQVVAIASHDGGKQALETVQRLLPVLCQAHGLTPEQVVAIASHDGGK
QALETVQRLLPVLCQAHGLTPEQVVAIASNGGGKQALETVQRLLPVLCQAHGLTPAQVVA
IASNGGGKQALETVQRLLPVLCQAHGLTPDQVVAIASNNGGKQALETVQRLLPVLCQDHG
LTPDQVVAIASNIGGKQALETVQRLLPVLCQDHGLTPAQVVAIASNNGGKQALETVQRLL
PVLCQDHGLTPAQVVAIASNIGGKQALETVQRLLPVLCQAHGLTPDQVVAIASNNGGKQA
LETVQRLLPVLCQDHGLTPAQVVAIASNIGGKQALETVQRLLPVLCQAHGLTPEQVVAIA
SNNGGKQALETVQRLLPVLCQAHGLTPEQVVAIASNGGGKQALESIVAQLSRPDPALAAL
TNDHLVALACLGGRPALDAVKKGLLVGSAIPVKRGATGETKVFTGNSNSPKSPTKGGCSG
SETPGTSESATPESSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVFGVR
NSKRGAAGSLMNVLNYPGMNHRVEITEGILADECAALLCDFYRMPRQVFNAQKKAQSSIN.
(The underlined portion corresponds to the TALE
protein including the N-terminal
domain and C-terminal domain.
(including the half domain))
SEQ ID NO: 123
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNIGGKQALETVQRLLPVLCQAHGLTPEQVVAIASNNGG
KQALETVQRLLPVLCQAHGLTPDQVVAIASNGGGKQALETVQRLLPVLCQDHGLTPDQVV
AIASHDGGKQALETVQRLLPVLCQAHGLTPAQVVAIASHDGGKQALETVQRLLPVLCQDH
GLTPAQVVAIASNGGGKQALETVQRLLPVLCQDHGLTPAQVVAIASNGGGKQALETVQRL
LPVLCQAHGLTPAQVVAIASNNGGKQALETVQRLLPVLCQDHGLTPAQVVAIASNIGGKQ
ALETVQRLLPVLCQAHGLTPDQVVAIASNNGGKQALETVQRLLPVLCQDHGLTPAQVVAI
ASNIGGKQALETVQRLLPVLCQAHGLTPEQVVAIASNNGGKQALETVQRLLPVLCQAHGL
TPAQVVAIASNIGGKQALETVQRLLPVLCQAHGLTPAQVVAIASNNGGKQALETVQRLLP
VLCQDHGLTPAQVVAIASNNGGKQALETVQRLLPVLCQAHGLTPAQVVAIASNIGGKQAL
ETVQRLLPVLCQAHGLTPDQVVAIASNGGGKQALETVQRLLPVLCQAHGLTPDQVVAIAS
NGGGKQALETVQRLLPVLCQAHGLTPEQVVAIASNGGGKQALESIVAQLSRPDPALAALT
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))
SEQ ID NO: 124
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPDQVVAIASNGGGKQALETVQRLLPVLCQAHGLTPAQVVAIASNGGG
KQALETVQRLLPVLCQAHGLTPEQVVAIASNGGGKQALETVQRLLPVLCQAHGLTPEQVV
AIASNNGGKQALETVQRLLPVLCQAHGLTPDQVVAIASNIGGKQALETVQRLLPVLCQDH
GLTPDQVVAIASNIGGKQALETVQRLLPVLCQAHGLTPAQVVAIASNNGGKQALETVQRL
LPVLCQDHGLTPAQVVAIASNGGGKQALETVQRLLPVLCQAHGLTPDQVVAIASHDGGKQ
ALETVQRLLPVLCQAHGLTPAQVVAIASHDGGKQALETVQRLLPVLCQAHGLTPDQVVAI
ASNGGGKQALETVQRLLPVLCQAHGLTPAQVVAIASNGGGKQALETVQRLLPVLCQDHGL
TPDQVVAIASNNGGKQALETVQRLLPVLCQDHGLTPAQVVAIASNIGGKQALETVQRLLP
VLCQAHGLTPEQVVAIASNNGGKQALETVQRLLPVLCQAHGLTPDQVVAIASNIGGKQAL
ETVQRLLPVLCQDHGLTPAQVVAIASNNGGKQALETVQRLLPVLCQDHGLTPAQVVAIAS
NIGGKQALETVQRLLPVLCQAHGLTPEQVVAIASNGGGKQALESIVAQLSRPDPALAALT
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))
SEQ ID NO: 125
MASVLTPLLLRGLTGSARRLPVPRAKIHSLDYKDHDGDYKDHDIDYKDDDDKGIRIQDLR
TLGYSQQQQEKIKPKVRSTVAQHHEALVGHGFTHAHIVALSQHPAALGTVAVKYQDMIAA
LPEATHEAIVGVGKYHGARALEALLTVAGELRGPPLQLDTGQLLKIAKRGGVTAVEAVHA
WRNALTGAPLNLTPAQVVAIASNNGGKQALETVQRLLPVLCQDHGLTPAQVVAIASNGGG
KQALETVQRLLPVLCQAHGLTPDQVVAIASHDGGKQALETVQRLLPVLCQDHGLTPAQVV
AIASHDGGKQALETVQRLLPVLCQAHGLTPDQVVAIASNGGGKQALETVQRLLPVLCQAH
GLTPAQVVAIASNGGGKQALETVQRLLPVLCQDHGLTPDQVVAIASNNGGKQALETVQRL
LPVLCQDHGLTPAQVVAIASNIGGKQALETVQRLLPVLCQAHGLTPEQVVAIASNNGGKQ
ALETVQRLLPVLCQAHGLTPDQVVAIASNIGGKQALETVQRLLPVLCQDHGLTPAQVVAI
ASNNGGKQALETVQRLLPVLCQDHGLTPAQVVAIASNIGGKQALETVQRLLPVLCQAHGL
TPAQVVAIASNNGGKQALETVQRLLPVLCQAHGLTPDQVVAIASNNGGKQALETVQRLLP
VLCQDHGLTPAQVVAIASNIGGKQALETVQRLLPVLCQAHGLTPEQVVAIASNGGGKQAL
ETVQRLLPVLCQAHGLTPDQVVAIASNGGGKQALETVQRLLPVLCQDHGLTPDQVVAIAS
NIGGKQALETVQRLLPVLCQDHGLTPEQVVAIASNGGGKQALESIVAQLSRPDPALAALI
NDHLVALACLGGRPALDAVKKGLLVGSAIPVKRGATGETKVFTGNSNSPKSPTKGGCSGS
ETPGTSESATPESSEVEFSHEYWMRHALTLAKRARDEREVPVGAVLVLNNRVIGEGWNRA
IGLHDPTAHAEIMALRQGGLVMQNYRLIDATLYVTFEPCVMCAGAMIHSRIGRVVFGVRN
SKRGAAGSLMNVLNYPGMNHRVEITEGILADECAALLCDFYRMPRQVFNAQKKAQSSIN.
(The underlined portion corresponds to the TALE
protein including the N-terminal domain
and C-terminal domain.
(including the half domain))

The combinations of the first fusion protein and the second fusion protein used in the base editors shown in FIG. 3 are as follows.

| Base Editor ID | First Fusion Protein | Second Fusion Protein |
|---|---|---|
| 306Nv + 120C | SEQ ID NO: 100 | SEQ ID NO: 118 |
| 310Nv + 385Cv | SEQ ID NO: 101 | SEQ ID NO: 119 |
| 310Nv + 120C | SEQ ID NO: 101 | SEQ ID NO: 118 |
| 314Nv + 120C | SEQ ID NO: 102 | SEQ ID NO: 118 |
| 318Nv + 397Cv | SEQ ID NO: 103 | SEQ ID NO: 120 |
| 318Nv + 385Cv | SEQ ID NO: 103 | SEQ ID NO: 119 |
| 318Nv + 120C | SEQ ID NO: 103 | SEQ ID NO: 118 |
| ZF6 97N + 120C | SEQ ID NO: 113 | SEQ ID NO: 118 |
| 322Nv + 385Cv | SEQ ID NO: 104 | SEQ ID NO: 119 |
| 322Nv + 120C | SEQ ID NO: 104 | SEQ ID NO: 118 |
| 322Nv + 115C | SEQ ID NO: 104 | SEQ ID NO: 121 |
| 322Nv + 110C | SEQ ID NO: 104 | SEQ ID NO: 122 |
| 90N + 397Cv | SEQ ID NO: 105 | SEQ ID NO: 120 |
| 90N + 389Cv | SEQ ID NO: 105 | SEQ ID NO: 123 |
| 90N + 385Cv | SEQ ID NO: 105 | SEQ ID NO: 119 |
| 90N + 120C | SEQ ID NO: 105 | SEQ ID NO: 118 |
| 90N + 115C | SEQ ID NO: 105 | SEQ ID NO: 121 |
| 90N + 110C | SEQ ID NO: 105 | SEQ ID NO: 122 |
| 326Nv + 385Cv | SEQ ID NO: 106 | SEQ ID NO: 119 |
| 326Nv + 120C | SEQ ID NO: 106 | SEQ ID NO: 118 |
| 326Nv + 115C | SEQ ID NO: 106 | SEQ ID NO: 121 |
| 326Nv + 110C | SEQ ID NO: 106 | SEQ ID NO: 122 |
| 326Nv + 381Cv | SEQ ID NO: 106 | SEQ ID NO: 124 |
| ZF1 97N + 397Cv | SEQ ID NO: 111 | SEQ ID NO: 120 |
| ZF1 97N + 120C | SEQ ID NO: 111 | SEQ ID NO: 118 |
| 330Nv + 120C | SEQ ID NO: 107 | SEQ ID NO: 118 |
| 334Nv + 397Cv | SEQ ID NO: 108 | SEQ ID NO: 120 |
| 334Nv + 385Cv | SEQ ID NO: 108 | SEQ ID NO: 119 |
| 334Nv + 120C | SEQ ID NO: 108 | SEQ ID NO: 118 |
| 334Nv + 115C | SEQ ID NO: 108 | SEQ ID NO: 121 |
| 334Nv + 110C | SEQ ID NO: 108 | SEQ ID NO: 122 |
| 334Nv + 381Cv | SEQ ID NO: 108 | SEQ ID NO: 124 |
| ZF5 97N + 397Cv | SEQ ID NO: 112 | SEQ ID NO: 120 |
| ZF5 97N + 120C | SEQ ID NO: 112 | SEQ ID NO: 118 |
| ZF5 97N + 115C | SEQ ID NO: 112 | SEQ ID NO: 121 |
| ZF5 97N + 381Cv | SEQ ID NO: 112 | SEQ ID NO: 124 |
| ZF6 97C + 389Nv | SEQ ID NO: 114 | SEQ ID NO: 115 |
| ZF6 97C + 385Nv | SEQ ID NO: 114 | SEQ ID NO: 116 |
| 322Cv + 393Nv | SEQ ID NO: 109 | SEQ ID NO: 117 |
| 322Cv + 389Nv | SEQ ID NO: 109 | SEQ ID NO: 115 |
| 322Cv + 385Nv | SEQ ID NO: 109 | SEQ ID NO: 116 |
| 90C + 389Nv | SEQ ID NO: 110 | SEQ ID NO: 115 |

1. A base editing composition capable of correcting a mitochondrial DNA mutation in a patient with Leber hereditary optic neuropathy (LHON), comprising:
one or more fusion proteins, wherein each of the one or more fusion proteins independently comprises DNA binding protein that specifically binds to mitochondrial DNA of a patient with LHON and further comprises at least one of adenine deaminase and cytosine deaminase, and
wherein cytosine deaminase is present in a full-length form or in the form of two splits.

2. The base editing composition of claim 1, wherein, in the patient with LHON, the composition is capable of editing:
adenine (A) at position 3460 of mitochondrial ND1 DNA to guanine (G),
adenine (A) at position 11778 of mitochondrial ND4 DNA to guanine (G), or
cytosine (C) at position 14484 of mitochondrial ND6 DNA to thymine (T).

3. The base editing composition of claim 1, wherein cytosine deaminase is apolipoprotein B editing complex (APOBEC), activation-induced deaminase (AID), tRNA-specific adenosine deaminase (TadA), or DddAₜₒₓ, or a variant thereof.

4. The base editing composition of claim 1, wherein cytosine interface deaminase is DddAₜₒₓ and is included in the form of a first split and a second split, and wherein one or more amino acids located on the interface between the first and second splits are substituted with other amino acids.

5. The base editing composition of claim 1, wherein adenine deaminase is TadA or a variant thereof.

6. The base editing composition of claim 5, wherein adenine deaminase comprises the amino acid sequence of SEQ ID NO: 1 or a conservative amino acid substitution thereof.

7. The base editing composition of claim 1, wherein DNA binding protein is selected from the group consisting of zinc finger protein, TALE protein, and CRISPR-associated nuclease.

8. The base editing composition of claim 1, wherein one DNA binding protein binds to a nucleotide sequence of 5′-TACGGGCTA CTACAACCCTTCGCTGACACCATAAAACTCTTCACCAAAGAGCCCCTAAA-3′ or a portion thereof of mitochondrial ND1 DNA.

9. The base editing composition of claim 1, wherein one DNA binding protein binds to a nucleotide sequence of 5′-CAAACTCAAACTACGAACGCACTCACAGTCACATCATAATCCTCTCTCAAGGACT TCAAAC-3′ or a portion thereof of mitochondrial ND4 DNA.

10. The base editing composition of claim 1, wherein one DNA binding protein binds to a nucleotide sequence of 5′-TCGCTGTAGTATATCCAAAGACAACCACCATTCCCCCTAAATAAATTAAAAAAAC T-3′ or a portion thereof mitochondrial ND6 DNA.

11. The base editing composition of claim 1, wherein the composition comprises two fusion proteins and is capable of editing adenine (A) at position 3460 of mitochondrial ND1 DNA to guanine (G) in a patient with LHON,
wherein each of the two fusion proteins comprises DddAₜₒₓ split and TALE protein that specifically binds to mitochondrial ND1 DNA, and
wherein one of the two fusion proteins further comprises TadA8e or a variant thereof.

12. The base editing composition of claim 1, wherein the composition comprises two fusion proteins and is capable of editing adenine (A) at position 11778 of mitochondrial ND4 DNA to guanine (G) in a patient with LHON,
wherein each of the two fusion proteins comprises DddAₜₒₓ split and TALE protein or zinc finger protein that specifically binds to mitochondrial ND4 DNA, and
wherein one of the two fusion proteins further comprises TadA8e or a variant thereof.

13. The base editing composition of claim 1, wherein the composition comprises two fusion proteins and is capable of editing cytosine (A) at position 14484 of mitochondrial ND6 DNA to thymine (T) in a patient with LHON,
wherein each of the two fusion proteins comprises DddAₜₒₓ split and TALE protein that specifically binds to mitochondrial ND6.
